(19) United States
(12) Patent Application Publication
(10) Pub. No.: US 2017/0202864 A1
(43) Pub. Date: Jul. 20, 2017

(54) NUTRIENT DELIVERY SYSTEM WITH HUMAN MILK OLIGOSACCHARIDES

(71) Applicant: ABBOTT LABORATORIES, Abbott Park, IL (US)

(72) Inventors: CHRISTINE GALLARDO, New Albany, OH (US); GARY GORDON, Plain City, OH (US); PAUL JOHNS, Columbus, OH (US); GARY KATZ, Columbus, OH (US); MARK ENGLE, Plain City, OH (US)

(73) Assignee: ABBOTT LABORATORIES, Abbott Park, IL (US)

(21) Appl. No.: 15/327,698

(22) PCT Filed: Jul. 21, 2015
(86) PCT No.: PCT/US15/41261

§ 371 Date: Jan. 20, 2017

Related U.S. Application Data
(60) Provisional application No. 62/026,807, filed on Jul. 21, 2014.

(51) Int. Cl. A61K 31/702; A23L 33/00; A61K 9/00; A23L 29/30; A61K 35/745; A61K 9/14

(52) U.S. Cl. CPC A61K 31/702; A61K 35/745; A61K 9/14; A61K 9/0053; A23L 29/30; A23L 33/40; A23V 2002/00

(57) ABSTRACT

Disclosed herein are nutrient delivery systems comprising a pod and a nutritional powder comprising a human milk oligosaccharide or a precursor thereof.

### CROSS-REFERENCE TO RELATED APPLICATIONS

This application claims the benefit of U.S. Provisional Patent Application No. 62/026,807, filed Jul. 21, 2014, the contents of which are herein fully incorporated by reference.

### TECHNICAL FIELD

The disclosure is directed to a nutrient delivery system comprising a pod and a nutritional powder. The nutritional powder includes at least one human milk oligosaccharide or a precursor thereof.

### BACKGROUND

Not all infants are in a position to receive human breast milk. It is therefore desirable to provide nutritional compositions, such as synthetic infant formulas, that can produce nutritional benefits in infants. Typical infant formulas are provided as packaged bulk powders, which a user must reconstitute by measuring out a specified amount of powder and adding it to an appropriate volume of liquid with shaking. This can lead to inconsistent amounts of both powder and liquid being used, and can lead to significant air being introduced into the formula when it is shaken. Furthermore, an infant formula is typically heated prior to consumption by the infant. However, heating infant formula through traditional methods can lead to deactivation of essential nutrients and vitamins. In addition, constant temperature monitoring is required to ensure that the infant formula is at the ideal temperature for easy consumption by the infant, leading to potential delays in providing an infant with formula in a timely manner.

To address the above concerns, it would be beneficial to provide nutritional compositions in an inexpensive and convenient manner, with all of the desirable nutrient deliverables at a proper temperature, and yet to provide the parent or caretaker a decrease in time for preparation. One such system is a single-serving pod system that could provide single bottle infant formulas in minutes with the proper nutrients for the infant, at the proper temperature. Issues related to proper reconstitution of the powder, lack of appropriate mixing in the pod, ensuring water safety, and minimizing or eliminating growth of microorganisms are challenges for current single-use pod systems for any type of liquid deliverable such as coffee or other nutrient formulations. Another issue is the expense and environmental impact of these single-serving pod systems.

Accordingly, there is a need for a single use pod system for providing nutritional compositions, such as infant formulas, which addresses the issues outlined above.

### SUMMARY

The present disclosure is directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the wherein the nutritional powder is comprised within the pod.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the nutritional formula is a synthetic infant formula.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide or precursor thereof is selected from the group consisting of sialic acid, 3′-sialyllactose, 6′-sialyllactose, 2′-fucosyllactose, 3′-fucosyllactose, lacto-N-tetraose and lacto-N-neotetraose, and any combination thereof.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide or precursor thereof is selected from the group consisting of sialic acid, 3′-sialyllactose, 6′-sialyllactose, 2′-fucosyllactose, 3′-fucosyllactose, lacto-N-tetraose and lacto-N-neotetraose, and any combination thereof, and wherein the human milk oligosaccharide or precursor thereof is a combination of human milk oligosaccharides selected from the group consisting of: a combination of 6′-sialyllactose and 3′-sialyllactose; a combination of 3′-fucosyllactose and sialic acid; a combination of 2′-fucosyllactose and 3′-fucosyllactose; a combination of 2′-fucosyllactose, 3′-sialyllactose, and 6′-sialyllactose; a combination of 3′-sialyllactose, 3′-fucosyllactose, and lacto-N-neotetraose; and a combination of 6′-sialyllactose, 2′-fucosyllactose, and lacto-N-neotetraose.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide or precursor thereof is selected from the group consisting of sialic acid, 3′-sialyllactose, 6′-sialyllactose, 2′-fucosyllactose, 3′-fucosyllactose, lacto-N-tetraose and lacto-N-neotetraose, and any combination thereof, and wherein the human milk oligosaccharide or precursor thereof is a combination of human milk oligosaccharides selected from the group consisting of: a combination of 6′-sialyllactose and 3′-sialyllactose; a combination of 3′-fucosyllactose and sialic acid; a combination of 2′-fucosyllactose and 3′-fucosyllactose; a combination of 2′-fucosyllactose, 3′-sialyllactose, and 6′-sialyllactose; a combination of 3′-sialyllactose, 3′-fucosyllactose, and lacto-N-neotetraose; and a combination of 6′-sialyllactose, 2′-fucosyllactose, and lacto-N-neotetraose, and wherein the combination of human milk oligosaccharides is present in the nutritional formula at a total concentration of from about 0.001 mg/mL to about 20 mg/mL.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is 6′-sialyllactose.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is 6′-sialyllactose, and wherein the 6′-sialyllactose is present in the nutritional formula at a concentration of from about 0.001 mg/mL to less than 0.25 mg/mL.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is 2′-fucosyllactose.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is 2′-fucosyllactose, and wherein the 2′-fucosyllactose is present in the nutritional formula at a concentration of from about 0.001 mg/mL to less than 2.0 mg/mL.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is 3′-fucosyllactose.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is 3′-fucosyllactose, wherein the concentration of 3′-fucosyllactose is from about 0.001 mg/mL to about 10 mg/mL.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is lacto-N-neotetraose.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the human milk oligosaccharide is lacto-N-neotetraose, and wherein the lacto-N-neotetraose is present in the nutritional formula at a concentration of from about 0.001 mg/mL to less than 0.2 mg/mL.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the nutritional powder further comprises a probiotic.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the nutritional powder further comprises a probiotic, wherein the probiotic is of human infant origin.

The present disclosure is also directed to a nutrient delivery system comprising: (a) a pod; and (b) a nutritional powder comprising a human milk oligosaccharide or a precursor thereof; wherein the nutrient delivery system provides a nutritional formula comprising the human milk oligosaccharide or precursor thereof, wherein the nutritional powder further comprises a probiotic, wherein the probiotic is of human infant origin, and wherein the probiotic is a Bifidobacterium.

The present disclosure is also directed to a method of promoting the growth of beneficial bacteria in an infant in need thereof, the method comprising administering to the infant an infant formula prepared from a nutrient delivery system comprising: (a) a pod; and (b) a nutritional powder comprising a human milk oligosaccharide or a precursor thereof

The present disclosure is also directed to a method of promoting the growth of beneficial bacteria in an infant in need thereof, the method comprising administering to the infant an infant formula prepared from a nutrient delivery system comprising: (a) a pod; and (b) a nutritional powder comprising a human milk oligosaccharide or a precursor thereof, wherein the beneficial bacteria is grown in the gastrointestinal tract of the infant.

The present disclosure is also directed to a method of promoting the growth of beneficial bacteria in an infant in need thereof, the method comprising administering to the infant an infant formula prepared from a nutrient delivery system comprising: (a) a pod; and (b) a nutritional powder comprising a human milk oligosaccharide or a precursor thereof, wherein the infant in need of beneficial bacteria suffers from necrotizing enterocolitis.

The present disclosure is also directed to a method of promoting the growth of beneficial bacteria in an infant in need thereof, the method comprising administering to the infant an infant formula prepared from a nutrient delivery system comprising: (a) a pod; and (b) a nutritional powder comprising a human milk oligosaccharide or a precursor thereof, wherein the infant in need of beneficial bacteria suffers from colitis.

The present disclosure is also directed to a method of stimulating enteric nerve cells in the gastrointestinal tract of an infant in need thereof, the method comprising administering to the infant an infant formula prepared from a nutrient delivery system comprising: (a) a pod; and (b) a nutritional powder comprising a human milk oligosaccharide or a precursor thereof.

The present disclosure is also directed to a method of improving cognition in an infant in need thereof, the method comprising administering to the infant an infant formula prepared from a nutrient delivery system comprising: (a) a pod; and (b) a nutritional powder comprising a human milk oligosaccharide or a precursor thereof.

The present disclosure is also directed to a method of promoting the growth of beneficial microbiota in the gastrointestinal tract of an infant in need thereof, the method comprising administering to the infant an infant formula prepared from a nutrient delivery system comprising: (a) a pod; and (b) a nutritional powder comprising a human milk oligosaccharide or a precursor thereof.

The present disclosure is also directed to a method of reducing the incidence of oxidative stress in an infant in need thereof, the method comprising administering to the infant an infant formula prepared from a nutrient delivery system comprising: (a) a pod; and (b) a nutritional powder comprising a human milk oligosaccharide or a precursor thereof.

The present disclosure is also directed to a method of reducing the incidence of necrotizing enterocolitis in an infant in need thereof, the method comprising administering to the infant an infant formula prepared from a nutrient delivery system comprising: (a) a pod; and (b) a nutritional powder comprising a human milk oligosaccharide or a precursor thereof

The present disclosure is also directed to a method of improving the feeding tolerance of an infant in need thereof, the method comprising administering to the infant an infant formula prepared from a nutrient delivery system comprising: (a) a pod; and (b) a nutritional powder comprising a human milk oligosaccharide or a precursor thereof.

The present disclosure is also directed to a method of reducing inflammation in an infant in need thereof, the method comprising administering to the infant an infant formula prepared from a nutrient delivery system comprising: (a) a pod; and (b) a nutritional powder comprising a human milk oligosaccharide or a precursor thereof.

The present disclosure is also directed to a method of inhibiting the growth of respiratory viruses in an infant in need thereof, the method comprising: identifying an infant having at least one of respiratory syncytial virus, human parainfluenza virus type 3, and influenza A virus; and administering to the infant an infant formula prepared from a nutrient delivery system comprising: (a) a pod; and (b) a nutritional powder comprising a human milk oligosaccharide or a precursor thereof.

The present disclosure is also directed to a method of improving airway respiratory health in an infant, toddler, or child in need thereof, the method comprising administering to the infant an infant formula prepared from a nutrient delivery system comprising: (a) a pod; and (b) a nutritional powder comprising a human milk oligosaccharide or a precursor thereof.

The present disclosure is also directed to a method of improving airway defense mechanisms in an infant, toddler, or child in need thereof, the method comprising administering to the infant an infant formula prepared from a nutrient delivery system comprising: (a) a pod; and (b) a nutritional powder comprising a human milk oligosaccharide or a precursor thereof.

### DETAILED DESCRIPTION

Disclosed herein is a nutrient delivery system that includes a pod and a nutritional powder, such as a synthetic infant formula powder, that can provide a nutritional formula (e.g., an infant formula) in an efficient and cost-effective manner. For example, the nutrient delivery system can be used in single-use applications, in which a single pod comprising a nutritional powder can be used to provide a single serving of a nutritional formula. Using the nutrient delivery system can provide an infant formula with a consistent formulation, providing an infant with an optimum amount of proper nutrients at an appropriate temperature.

The nutritional powders for use in the pod of the nutrient delivery system include one or more human milk oligosaccharides (HMOs). The nutritional powders and formulas, as well as methods described herein, use HMOs alone or in combination with at least one other prebiotic oligosaccharide and/or a probiotic for controlling and reducing a number of diseases, disorders and conditions related to the gut-brain-immune system. Using the HMOs in the nutrient delivery system allows for consistent and accurate dosing of HMOs to an infant in a nutritional formula such as an infant formula. Additionally, using the HMOs may impart bacteriostatic and/or anti-microbial properties to a nutritional powder or a nutritional formula.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The terms “adult formula” and “adult nutritional product” as used herein are used interchangeably to refer to nutritional compositions for generally maintaining or improving the health of an adult.

The terms “bioavailable” or “bioavailability” as used herein, unless otherwise specified, refer to the amount of a nutrient made available to target tissues in a subject through the systemic circulation in the subject's body. In this context, the terms “bioavailable” or “bioavailability” may specifically refer to the ability of a lipophilic nutrient, such as Vitamin D, to be absorbed from the gastrointestinal tract into lymph which will then enter into the bloodstream of an individual such that the substance can be absorbed into organs and tissues in the body. As the degree of bioavailability of a nutrient increases, the nutrient becomes more likely to enter into and remain in the bloodstream where it can be absorbed and used by the body. As the degree of bioavailability of a nutrient decreases, the nutrient becomes less likely to be absorbed into lymph from the gastrointestinal tract and instead is excreted from the body before entering the bloodstream.

The term “composition” as used herein, unless otherwise specified, refers to mixtures that are suitable for enteral administration to a subject. Compositions may be in the form of powders, solids, semi-solids, liquids, gels, and semi-liquids. Compositions may further comprise vitamins, minerals, and other ingredients.

The term “dry blended” as used herein, unless otherwise specified, refers to the mixing of dry or semi-dry components or ingredients to form a base powder, or to the addition of a dry, powdered or granulated component or ingredient to a base powder, to form a powdered composition.

The terms “enteral administration” or “enterally administering” as used herein refer to providing a composition that is ingested by the subject through the gastrointestinal tract, e.g., orally or through a feeding tube into the stomach. This is in contrast to parenteral administration, which occurs through means other than the gastrointestinal tract, e.g., intravenously.

The terms “fat,” “lipid,” and “oil” as used herein, unless otherwise specified, are used interchangeably to refer to lipid materials derived or processed from plants or animals. These terms also include synthetic lipid materials so long as such synthetic materials are suitable for administration to subjects as defined herein.

The term “fatty acids” as used herein refers generally to carboxylic acids with long lipophilic chains comprising carbon and hydrogen atoms. Specific fatty acids can be identified by counting the number of carbon atoms and determining other chemical properties, such as the presence and location of double bonds between the carbon atoms, any branching of carbon atoms off the main lipophilic chain, and the presence of other atomic species in the chain. Fatty acids may be described as “saturated” (no double bonds between the carbon atoms), “monounsaturated” (one double bond between the carbon atoms), or “polyunsaturated” (more than one double bond between the carbon atoms). For the purpose of this disclosure, “free fatty acids” refer to unbonded fatty acid molecules, while “fatty acid groups” refer to fatty acid moieties bonded to other molecules. For the purpose of this disclosure, fatty acid groups are preferably bonded to glycerol molecules to form glycerides. For the purpose of this disclosure, “fatty acids” refers to both free fatty acids and fatty acid groups in a composition, unless otherwise specified.

The term “glycerides” as used herein refer generally to lipophilic compounds comprising a glycerol molecule bonded to fatty acid groups. Monoglycerides are glycerol molecules bonded to a single fatty acid group; diglycerides are glycerol molecules bonded to two fatty acid groups; and triglycerides are glycerol molecules bonded to three fatty acid groups. Fats and oils comprise glycerides, and typical fats and oils from animal, fish, algae, vegetable, or seed sources are comprised primarily of triglycerides.

The terms “growth of a virus” or “growth of bacteria” as used herein, unless otherwise specified, refer to the production, proliferation, or replication of a virus or bacteria.

The term “human milk fortifier” as used herein, unless otherwise specified, refers to compositions suitable for mixing with breast milk, or infant formula for consumption by an infant.

The terms “human milk oligosaccharide” or “HMO”, unless otherwise specified, refer generally to a number of complex carbohydrates found in human breast milk that can be in acidic or neutral form, and to precursors thereof Exemplary non-limiting human milk oligosaccharides include 3′-sialyllactose, 6′-sialyllactose, 3′-fucosyllactose, 2′-fucosyllactose, and lacto-N-neotetraose. Exemplary human milk oligosaccharide precursors include sialic acid and/or fucose.

The term “infant,” as used herein, unless otherwise specified, refers to a human about 12 months of age or younger. The term “toddler,” as used herein, unless otherwise specified, refers to a human about 12 months of age to about 3 years of age. The term “child,” as used herein, unless otherwise specified, refers to a human about 3 years of age to about 18 years of age. The term “adult,” as used herein, unless otherwise specified, refers to a human about 18 years of age or older.

The terms “infant formula” or “infant nutritional product” as used herein are used interchangeably to refer to nutritional compositions that have the proper balance of macronutrients, micro-nutrients, and calories to provide sole or supplemental nourishment for and generally maintain or improve the health of infants, toddlers, or both. Infant formulas preferably comprise nutrients in accordance with the relevant infant formula guidelines for the targeted consumer or user population, an example of which would be the Infant Formula Act, 21 U.S.C. Section 350(a).

The term “lipophilic nutrient” as used herein refers to components that have greater solubility in organic solvents such as ethanol, methanol, ethyl ether, acetone, chloroform, benzene, or lipids than they have in water. Vitamin D is one example of a lipophilic nutrient. For the purpose of this disclosure, the term “lipophilic nutrient” may be applied to other lipophilic compounds, including but not limited to pharmaceutical compounds.

The term “liquid composition” as used herein refers to compositions in ready-to-consume liquid form or concentrated liquid form.

The terms “liquid nutritional composition” and “nutritional liquid” as used herein are used interchangeably to refer to nutritional products in ready-to-consume liquid form or concentrated liquid form.

The term “nutritional composition” as used herein, unless otherwise specified, refers to nutritional powders, solids, semi-solids, liquids, and semi-liquids that comprise at least one of protein, carbohydrate, and lipid, and are suitable for enteral administration to a subject. Nutritional compositions may further comprise vitamins, minerals, and other ingredients, and represent sole, primary, or supplemental sources of nutrition.

The term “nutritional formula” as used herein, unless otherwise specified, refers to nutritional compositions in ready-to-drink liquid form, concentrated form, and nutritional liquids made by reconstituting the nutritional powders described herein, wherein the powder can be completely dissolved, partially dissolved, mixed, suspended or any combination thereof, prior to use. The formula may be completely homogeneous or partially homogeneous, and may be a solution, a homogeneous suspension, an emulsion, a homogeneous dispersion, or any combination thereof.

The term “nutritional powder” as used herein, unless otherwise specified, refers to nutritional products in flowable or scoopable form that can be reconstituted with water or another aqueous liquid prior to consumption and includes both spray-dried, dry-mixed/dry-blended, and extruded powders.

The terms “pediatric formula” or “pediatric nutritional product,” as used herein, are used interchangeably to refer to nutritional compositions for generally maintaining or improving the health of infants and children.

The term “powder” as used herein describes a physical form of a composition, or portion thereof, that is a finely divided particulate solid that is flowable or scoopable.

The term “reconstitute” as used herein, unless otherwise specified, refers to a process in which a powder such as a nutritional powder is mixed with a liquid, such as water or another aqueous liquid to create a liquid composition that is essentially homogeneous. The reconstituted composition, such as a nutritional formula (e.g., an infant formula) may be completely homogeneous or partially homogeneous. The reconstituted composition may be a solution, a homogeneous suspension, an emulsion, a homogeneous dispersion, or any combination thereof.

The term “serving” as used herein, unless otherwise specified, is any amount of a composition that is intended to be ingested by a subject in one sitting or within less than about one hour. The size of a serving (i.e., “serving size”) may be different for diverse subjects, depending on one or more factors including, but not limited to, age, body mass, gender, species, or health. For a typical human adult, a serving size of the formulas disclosed herein is from about 25 mL to 1,000 mL. For a typical human infant or baby, a serving size of the formulas disclosed herein is from about 5 mL to about 250 mL.

The term “shelf life” as used herein, unless otherwise specified, refers to the time that a nutritional product such as a formula or powder remains commercially stable after being packaged and then stored at 18-30° C. (e.g., 18° C., 19° C., 20° C., 21° C., 22° C., 23° C., 24° C., 25° C., 26° C., 27° C., 28° C., 29° C., 30° C.). A nutritional product may have a shelf life of at least 1 month, at least 3 months, at least 6 months, at least 12 months, at least 18 months, at least 24 months, or at least 36 months, including from about 1 month to about 36 months, 3 months to about 36 months, 6 months to about 36 months, 12 months to about 36 months, 18 months to about 36 months, 24 months to about 36 months, 1 month to about 24 months, 3 months to about 24 months, 6 months to about 24 months, 12 months to about 24 months, 18 months to about 24 months, 1 month to about 18 months, 3 months to about 18 months, 6 months to about 18 months, 12 months to about 18 months, 1 month to about 12 months, 3 months to about 12 months, 6 months to about 12 months, or up to 36 months.

The term “subject” as used herein refers to a mammal, including but not limited to a human (e.g., an infant, toddler, child or adult), a domesticated farm animal (e.g., cow, horse, or pig), or a pet (e.g., dog or cat), who ingests the composition.

“Total protein” and “total amount of protein” are used interchangeably in connection with the amount of protein in a protein system or a particular nutritional composition to mean all the protein in that system or composition.

To the extent that the terms “includes,” “including,” “contains,” or “containing” are used herein, they are intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used herein, they are intended to additionally mean “on” or “onto.”

All percentages, parts and ratios as used herein are by weight of the total product, unless specified otherwise. All such weights as they pertain to listed ingredients are based on the active ingredients and, therefore, do not include solvents or by-products that may be included in commercially available materials, unless specified otherwise.

All references to singular characteristics or limitations of the present disclosure shall include the corresponding plural characteristics or limitations, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the powders and formulas of the present disclosure may include trace amounts of any optional or selected essential ingredient or feature described herein, provided that the remaining composition (e.g., powder or formula) still contains all of the required ingredients or features as described herein. In this context, and unless otherwise specified, the term “trace amount” means that the selected composition (e.g., powder or formula) contains no more than 2 wt % of the optional ingredient, typically less than 1 wt %, and also includes zero percent, of such optional or selected essential ingredient, by weight of the composition.

The various embodiments of the powders and formulas of the present disclosure may also be substantially free of any optional ingredient or feature described herein, provided that the remaining composition still contains all of the required ingredients or features as described herein. In this context, and unless otherwise specified, the term “substantially free” means that the selected composition contains less than a functional amount of the optional ingredient, typically less than about 1 wt %, including less than about 0.5 wt %, including less than about 0.1 wt %, and also including zero percent, of such optional ingredient, by weight of the composition.

The powders and formulas may comprise, consist of, or consist essentially of the required elements of the products as described herein, as well as any additional or optional element described herein or otherwise useful in product applications.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. NUTRIENT DELIVERY SYSTEM

Provided herein is a nutrient delivery system. This system includes a nutritional powder, and the system provides a nutritional formula for consumption. This nutritional formula may be ingested by an infant and thus, provides the infant nutrients needed for proper development and growth. The nutritional formula may also be ingested by toddler or child, for proper delivery of nutrients for continued development and growth. The nutritional formula may also be ingested by an adult, as a nutritional supplement. The system also includes a pod, which contains the nutritional powder. The nutritional powder may be contained in the pod such that a headspace in the pod includes a maximum of about 10% O₂ (i.e. less than or equal to about 10% O₂), thereby reducing oxidation of the nutritional powder or formula and preventing the development of undesirable flavors, smells, and textures. Prior to ingestion of the nutritional formula, water is introduced into the pod to form a mixture of the water and the nutritional powder, ultimately providing the nutritional formula. The temperature of the water may be about 5° C. to 60° C., e.g., about 25° C. to about 50° C., to allow reconstitution of the nutritional powder to provide the nutritional formula. For example, the temperature of the water may be about 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C. The resulting nutritional formula is then discharged from the pod into a container suitable for facilitating consumption of the nutritional formula by an individual (e.g., infant, toddler, child or adult).

a. Nutritional Powder

The nutrient delivery system may comprise a nutritional powder that is within a pod and delivers a nutritional formula. The nutritional powder includes compounds that affect the overall physical characteristics of the nutritional formula. The nutritional powder is sealed in the pod and is measured in amount that provides the optional nutritional formula when used in the nutrient delivery system. The physical characteristics that are important for the overall function of the nutritional powder include powder reconstitution characteristics (e.g., wettability), viscosity, foaming, emulsion stability, amino acid profile, mineral delivery, antioxidant capacity, shelf-life stability, odor, flavor, and digestibility.

(1) Size and Shape

The nutritional powder may comprise a particle size distribution of about 1 μm to about 1000 μm. The particle size of the nutritional powder is a significant factor determining the wettability and flow properties of the nutritional formula. The nutritional powder mean particle size may be measured by particle size analysis techniques that include, but are not limited to, laser diffraction, sieve separation analysis and image analysis (e.g., using a microscopic method such as light microscopy or scanning electron microscopy). The nutritional powder mean particle size may be from about 1 μm to about 1000 μm, about 10 μm to about 700 μm, about 20 μm to about 600 μm, about 30 μm to about 500 μm, about 40 μm to about 400 μm, about 30 μm to about 300 μm, about 60 μm to about 200 μm, about 80 μm to about 200 μm, or about 100 μm to about 190 μm. The nutritional powder mean particle size may be about 1 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, 700 μm, 725 μm, 750 μm, 775 μm, 800 μm, 825 μm, 850 μm, 875 μm, 900 μm, 925 μm, 950 μm, 975 μm, or 1000 μm.

The nutritional powder may comprise particles of variable shapes. The shape of the particles differs from size of the particles by describing the external boundaries and surface of the particles. The shape and size of the nutritional composition particles can be used together to better characterize the nutritional powder. The shape of the nutritional powder is important in determining the wettability and flow properties of the formula. The nutritional powder particle shape and/or distribution of particle shapes may be determined by laser diffraction, and image analysis (e.g., using a microscopic method such as light microscopy or scanning electron microscopy). For example, size shape and morphology may be ascertained using a Malvern Morphologi G3, or other similar equipment used within the art. Using the above techniques it may be possible to determine statistical numbers for surface roughness, solidity and/or ruggedness. The aspect ratio (length divided by width), circularity, convexity, elongation, high sensitivity (HS) circularity, solidity fiber elongation, and fiber straightness can also be determined. The shape of the nutritional powder may be, but is not limited to, sphere, cube, plate, flake, rod or thread, or any combination thereof In some embodiments, the nutritional powder may include irregularly shaped particles.

The nutritional powder may comprise particles of an aspect ratio of about 0.1 to about 1.0. The aspect ratio is a value which can aid in the analyzing the particle shapes comprised within the nutritional powder. The aspect ratio of the nutritional powder particles can affect the wettability and flow properties of the formula. The nutritional powder particle aspect ratio may be determined by laser diffraction, and image analysis. For example, particle aspect ratio may be ascertained using a Malvern Morphologi G3, or other similar equipment used within the art. The particles of the nutritional powder may have an aspect ratio of about 0.01, 0.03, 0.05, 0.07, 0.09, 0.11, 0.13, 0.15, 0.17, 0.19, 0.21, 0.23, 0.25, 0.27, 0.29, 0.31, 0.33, 0.35, 0.37, 0.39, 0.41, 0.43, 0.45, 0.47, 0.49, 0.51, 0.53, 0.55, 0.57, 0.59, 0.61, 0.63, 0.65, 0.67, 0.69, 0.71, 0.73, 0.75, 0.77, 0.79, 0.81, 0.83, 0.85, 0.87, 0.89, 0.91, 0.93, 0.95, 0.97, 0.99, or 1.

(2) Density

The nutritional powder may comprise a loose bulk density of about 0.2 g/mL to about 1.0 g/mL. The loose bulk density of said powder quantifies the density of the powder without vibration. The loose bulk density may be examined by measuring the mass of a known volume of nutritional powder. The loose bulk density of the nutritional powder may be about 0.20 g/mL, 0.205 g/mL, 0.21 g/mL, 0.215 g/mL, 0.22 g/mL, 0.225 g/mL, 0.23 g/mL, 0.235 g/mL, 0.24 g/mL, 0.245 g/mL, 0.25 g/mL, 0.255 g/mL, 0.26 g/mL, 0.265 g/mL, 0.27 g/mL, 0.275 g/mL, 0.28 g/mL, 0.285 g/mL, 0.29 g/mL, 0.295 g/mL, 0.30 g/mL, 0.305 g/mL, 0.31 g/mL, 0.315 g/mL, 0.32 g/mL, 0.325 g/mL, 0.33 g/mL, 0.335 g/mL, 0.34 g/mL, 0.345 g/mL, 0.35 g/mL, 0.355 g/mL, 0.36 g/mL, 0.365 g/mL, 0.37 g/mL, 0.375 g/mL, 0.38 g/mL, 0.385 g/mL, 0.39 g/mL, 0.395 g/mL, 0.40 g/mL, 0.405 g/mL, 0.41 g/mL, 0.415 g/mL, 0.42 g/mL, 0.425 g/mL, 0.43 g/mL, 0.435 g/mL, 0.44 g/mL, 0.445 g/mL, 0.45 g/mL, 0.455 g/mL, 0.46 g/mL, 0.465 g/mL, 0.47 g/mL, 0.475 g/mL, 0.48 g/mL, 0.485 g/mL, 0.49 g/mL, 0.495 g/mL, 0.50 g/mL, 0.505 g/mL, 0.51 g/mL, 0.515 g/mL, 0.52 g/mL, 0.525 g/mL, 0.53 g/mL, 0.535 g/mL, 0.54 g/mL, 0.545 g/mL, 0.55 g/mL, 0.555 g/mL, 0.56 g/mL, 0.565 g/mL, 0.57 g/mL, 0.575 g/mL, 0.58 g/mL, 0.585 g/mL, 0.59 g/mL, 0.595 g/mL, 0.60 g/mL, 0.605 g/mL, 0.61 g/mL, 0.615 g/mL, 0.62 g/mL, 0.625 g/mL, 0.63 g/mL, 0.635 g/mL, 0.64 g/mL, 0.645 g/mL, 0.65 g/mL, 0.655 g/mL, 0.66 g/mL, 0.665 g/mL, 0.67 g/mL, 0.675 g/mL, 0.68 g/mL, 0.685 g/mL, 0.69 g/mL, 0.695 g/mL, 0.70 g/mL, 0.705 g/mL, 0.71 g/mL, 0.715 g/mL, 0.72 g/mL, 0.725 g/mL, 0.73 g/mL, 0.735 g/mL, 0.74 g/mL, 0.745 g/mL, 0.75 g/mL, 0.755 g/mL, 0.76 g/mL, 0.765 g/mL, 0.77 g/mL, 0.775 g/mL, 0.78 g/mL, 0.785 g/mL, 0.79 g/mL, 0.795 g/mL, 0.80 g/mL, 0.805 g/mL, 0.81 g/mL, 0.815 g/mL, 0.82 g/mL, 0.825 g/mL, 0.83 g/mL, 0.835 g/mL, 0.84 g/mL, 0.845 g/mL, 0.85 g/mL, 0.855 g/mL, 0.86 g/mL, 0.865 g/mL, 0.87 g/mL, 0.875 g/mL, 0.88 g/mL, 0.885 g/mL, 0.89 g/mL, 0.895 g/mL, 0.90 g/mL, 0.905 g/mL, 0.91 g/mL, 0.915 g/mL, 0.92 g/mL, 0.925 g/mL, 0.93 g/mL, 0.935 g/mL, 0.94 g/mL, 0.945 g/mL, 0.95 g/mL, 0.955 g/mL, 0.96 g/mL, 0.965 g/mL, 0.97 g/mL, 0.975 g/mL, 0.98 g/mL, 0.985 g/mL, 0.99 g/mL, 0.995 g/mL or 1.0 g/mL.

The nutritional powder may comprise a vibrated bulk density of 0.2 g/mL to 1.0 g/mL. The vibrated bulk density quantifies the density of a powder that has been subjected to vibration over a period of a time. The vibrated bulk density may be examined by measuring the mass of a known volume of nutritional powder, after undergoing at least once vibrational cycle. The vibrated bulk density of the nutritional powder may be about 0.20 g/mL, 0.205 g/mL, 0.21 g/mL, 0.215 g/mL, 0.22 g/mL, 0.225 g/mL, 0.23 g/mL, 0.235 g/mL, 0.24 g/mL, 0.245 g/mL, 0.25 g/mL, 0.255 g/mL, 0.26 g/mL, 0.265 g/mL, 0.27 g/mL, 0.275 g/mL, 0.28 g/mL, 0.285 g/mL, 0.29 g/mL, 0.295 g/mL, 0.30 g/mL, 0.305 g/mL, 0.31 g/mL, 0.315 g/mL, 0.32 g/mL, 0.325 g/mL, 0.33 g/mL, 0.335 g/mL, 0.34 g/mL, 0.345 g/mL, 0.35 g/mL, 0.355 g/mL, 0.36 g/mL, 0.365 g/mL, 0.37 g/mL, 0.375 g/mL, 0.38 g/mL, 0.385 g/mL, 0.39 g/mL, 0.395 g/mL, 0.40 g/mL, 0.405 g/mL, 0.41 g/mL, 0.415 g/mL, 0.42 g/mL, 0.425 g/mL, 0.43 g/mL, 0.435 g/mL, 0.44 g/mL, 0.445 g/mL, 0.45 g/mL, 0.455 g/mL, 0.46 g/mL, 0.465 g/mL, 0.47 g/mL, 0.475 g/mL, 0.48 g/mL, 0.485 g/mL, 0.49 g/mL, 0.495 g/mL, 0.50 g/mL, 0.505 g/mL, 0.51 g/mL, 0.515 g/mL, 0.52 g/mL, 0.525 g/mL, 0.53 g/mL, 0.535 g/mL, 0.54 g/mL, 0.545 g/mL, 0.55 g/mL, 0.555 g/mL, 0.56 g/mL, 0.565 g/mL, 0.57 g/mL, 0.575 g/mL, 0.58 g/mL, 0.585 g/mL, 0.59 g/mL, 0.595 g/mL, 0.60 g/mL, 0.605 g/mL, 0.61 g/mL, 0.615 g/mL, 0.62 g/mL, 0.625 g/mL, 0.63 g/mL, 0.635 g/mL, 0.64 g/mL, 0.645 g/mL, 0.65 g/mL, 0.655 g/mL, 0.66 g/mL, 0.665 g/mL, 0.67 g/mL, 0.675 g/mL, 0.68 g/mL, 0.685 g/mL, 0.69 g/mL, 0.695 g/mL, 0.70 g/mL, 0.705 g/mL, 0.71 g/mL, 0.715 g/mL, 0.72 g/mL, 0.725 g/mL, 0.73 g/mL, 0.735 g/mL, 0.74 g/mL, 0.745 g/mL, 0.75 g/mL, 0.755 g/mL, 0.76 g/mL, 0.765 g/mL, 0.77 g/mL, 0.775 g/mL, 0.78 g/mL, 0.785 g/mL, 0.79 g/mL, 0.795 g/mL, 0.80 g/mL, 0.805 g/mL, 0.81 g/mL, 0.815 g/mL, 0.82 g/mL, 0.825 g/mL, 0.83 g/mL, 0.835 g/mL, 0.84 g/mL, 0.845 g/mL, 0.85 g/mL, 0.855 g/mL, 0.86 g/mL, 0.865 g/mL, 0.87 g/mL, 0.875 g/mL, 0.88 g/mL, 0.885 g/mL, 0.89 g/mL, 0.895 g/mL, 0.90 g/mL, 0.905 g/mL, 0.91 g/mL, 0.915 g/mL, 0.92 g/mL, 0.925 g/mL, 0.93 g/mL, 0.935 g/mL, 0.94 g/mL, 0.945 g/mL, 0.95 g/mL, 0.955 g/mL, 0.96 g/mL, 0.965 g/mL, 0.97 g/mL, 0.975 g/mL, 0.98 g/mL, 0.985 g/mL, 0.99 g/mL, 0.995 g/mL, or 1.0 g/mL.

(3) Surface Area

The nutritional powder may comprise particles with a surface of about 0.02 m²/g to about 3.0 m²/g. The surface area of the particles within the nutritional powder is dependent on the size, shape and porosity of said particles, and is important in determining properties of the nutritional formula, such as dispersibility and rate of reconstitution. The particle porosity of the nutritional powder may be examined by the intrusion of a non-wetting liquid (e.g., mercury) at high pressure into the powder through the use of a porosimeter. The pore size can be determined based on the external pressure needed to force the liquid into a pore against the opposing force of the liquid's surface tension. The particles of the nutritional powder may have a surface area of about 0.02 m²/g, 0.04 m²/g, 0.06 m²/g, 0.08 m²/g, 0.10 m²/g, 0.15 m²/g, 0.20 m²/g, 0.25 m²/g, 0.30 m²/g, 0.35 m²/g, 0.40 m²/g, 0.45 m²/g, 0.50 m²/g, 0.55 m²/g, 0.60 m²/g, 0.65 m²/g, 0.70 m²/g, 0.75 m²/g, 0.80 m²/g, 0.85 m²/g, 0.90 m²/g, 0.95 m²/g, 1.0 m²/g, 1.05 m²/g, 1.1 m²/g, 1.15 m²/g, 1.2 m²/g, 1.25 m²/g, 1.3 m²/g, 1.35 m²/g, 1.4 m²/g, 1.45 m²/g, 1.5 m²/g, 1.55 m²/g, 1.6 m²/g, 1.65 m²/g, 1.7 m²/g, 1.75 m²/g, 1.8 m²/g, 1.85 m²/g, 1.9 m²/g, 1.95 m²/g, 2.0 m²/g, 2.05 m²/g, 2.1 m²/g, 2.15 m²/g, 2.2 m²/g, 2.25 m²/g, 2.3 m²/g, 2.35 m²/g, 2.4 m²/g, 2.45 m²/g, 2.5 m²/g, 2.55 m²/g, 2.6 m²/g, 2.65 m²/g, 2.7 m²/g, 2.75 m²/g, 2.8 m²/g, 2.85 m²/g, 2.9 m²/g, 2.95 m²/g, or 3.0 m²/g.

(4) Glass and Melt Transition Temperatures

The nutritional powder may comprise a glass transition temperature of about 30° C. to about 90° C. The glass transition temperature of the nutritional powder describes the liquification of said powder. Upon heating, the amorphous domain in a material will change from a glassy state to a rubbery state, while the crystalline domain will liquefy from a solid to a liquid. Glass transition analysis is useful as a comparison for a new product at standard moisture range. The glass transition temperature of a nutritional powder may be investigated via Differential Scanning calorimetry. The glass transition temperature of the nutritional powder may be about 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C.

The nutritional powder may comprise a melt transition temperature of about 40° C. to about 100° C. The melting temperature of the nutritional powder describes the liquification of said powder upon heating the powder, which has the ability to form crystalline domains, in a solid state to one in the fluid state. The nutritional powder may comprise a melting point when it is composed of solid fats versus liquid fats, which are more likely to instill some crystallinity within the powder. Melting temperature analysis is useful as a comparison for a new product at standard moisture range. The melting temperature of a nutritional powder may be investigated via Differential Scanning calorimetry. The melting temperature of the nutritional powder may be about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

(5) Flowability

The nutritional powder may comprise a flow factor of about 1 to about 10. The flowability of the nutritional powder is important in determining flow properties of the nutritional formula, such as rate of reconstitution and dispersibility. The flowability of the nutritional powder is a function of the nutritional powder particle characteristics, as well as the compounds within the powder, and is a measurement of the cohesion property of the nutritional powder. Flowability may be measured by a Brookfield powder flow tester, and is reported as a value of the flow factor and flow index. Flow factor is defined as the ratio of major principal consolidating stress (y-axis) to unconfined failure strength (x-axis) at 10 kPa of x-axis. Flow index is the inverse of flow factor. The flow factor of the nutritional powder may be about 1 to 10 or 1 to 8; for example the flow factor may be about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10.

(6) Particle Porosity

The nutritional powder may comprise a particle porosity of about 5% to about 80%. The porosity of nutritional powder particles is important in determining the wettability and flow properties of the composition. The porosity of the nutritional powder particles may be measured by determining the volume of the open pores and interstitial void divided by the envelope powder volume, providing values in units of percent (from 0-100%). For example, the porosity of the nutritional powder particles may be about 5 to 80%, about 10% to about 80%, about 15% to about 80%, about 20% to about 80%, about 25% to about 80%, about 30% to about 80%, about 35% to about 80%, or about 40% to about 75%. The porosity of the nutritional powder particles may be about 5%, 6%, 7%, 8%, 9%, 10%, 11%, 12%, 13%, 14%, 15%, 16%, 17%, 18%, 19%, 20%, 21%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80%.

(7) Wettability

The nutritional powder may comprise a wettability of about 1 second to about 180 seconds, or about 1 second to about 30 seconds. The wettability of the nutritional powder is important on the overall flow performance of the nutritional formula through the nutrient delivery system. The wettability of the nutritional powder may be measured indirectly by adding a powder to the surface of water in a container (e.g., a beaker) and recording the time it takes for the powder to fall below the surface. The wettability may be about 1 second to about 20 seconds, or about 2 seconds to about 10 seconds. For example, the wettability may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 seconds.

(8) Free Fat

The nutritional powder may comprise free fat of about 0.1 g/100 g powder to about 12 g/100 g powder. High levels of free fat in the nutritional powder can be detrimental to the flowability of the powder, and potentially lead to difficulties in providing the nutritional formula. The free fat within the nutritional powder may be determined by performing a hexane (or other suitable non-polar solvents, for example petroleum ether) extraction, followed by filtration (e.g., Whatman No. 41 filter paper) of the solvent extract (to remove suspended powder particles), drying oven evaporation of the solvent from the filtrate (e.g., at 60° C. for 2 hours) and weighing of the non-volatile residue (i.e., the extracted free fat) from the filtrate. The nutritional powder may comprise a free fat of about 0.1 g/100 g powder, 0.3 g/100 g powder, 0.5 g/100 g powder, 0.7 g/100 g powder, 0.9 g/100 g powder, 1.1 g/100 g powder, 1.3 g/100 g powder, 1.5 g/100 g powder, 1.7 g/100 g powder, 1.9 g/100 g powder, 2.1 g/100 g powder, 2.3 g/100 g powder, 2.5 g/100 g powder, 2.7 g/100 g powder, 2.9 g/100 g powder, 3.1 g/100 g powder, 3.3 g/100 g powder, 3.5 g/100 g powder, 3.7 g/100 g powder, 3.9 g/100 g powder, 4.1 g/100 g powder, 4.3 g/100 g powder, 4.5 g/100 g powder, 4.7 g/100 g powder, 4.9 g/100 g powder, 5.1 g/100 g powder, 5.3 g/100 g powder, 5.5 g/100 g powder, 5.7 g/100 g powder, 5.9 g/100 g powder, 6.1 g/100 g powder, 6.3 g/100 g powder, 6.5 g/100 g powder, 6.7 g/100 g powder, 6.9 g/100 g powder, 7.1 g/100 g powder, 7.3 g/100 g powder, 7.5 g/100 g powder, 7.7 g/100 g powder, 7.9 g/100 g powder, 8.1 g/100 g powder, 8.3 g/100 g powder, 8.5 g/100 g powder, 8.7 g/100 g powder, 8.9 g/100 g powder, 9.1 g/100 g powder, 9.3 g/100 g powder, 9.5 g/100 g powder, 9.7 g/100 g powder, 9.9 g/100 g powder, 10.1 g/100 g powder, 10.3 g/100 g powder, 10.5 g/100 g powder, 10.7 g/100 g powder, 10.9 g/100 g powder, 11.1 g/100 g powder, 11.3 g/100 g powder, 11.5 g/100 g powder, 11.7 g/100 g powder, 11.9 g/100 g powder, 12.1 g/100 g powder, 12.3 g/100 g powder, 12.5 g/100 g powder, 12.7 g/100 g powder, 12.9 g/100 g powder, 13.1 g/100 g powder, 13.3 g/100 g powder, 13.5 g/100 g powder, 13.7 g/100 g powder, 13.9 g/100 g powder, 14.1 g/100 g powder, 14.3 g/100 g powder, 14.5 g/100 g powder, 14.7 g/100 g powder, 14.9 g/100 g powder, or 15 g/100 g powder.

(9) Reconstitution

The nutritional powder may comprise a percent of reconstitution of about 75% to about 100%. The percent of reconstitution is important in determining the flow characteristics of the formula through the nutrient delivery system. The percent of reconstitution of the nutritional powder is dependent on properties of the nutritional powder such as, powder particle size, porosity and shape. The percent of reconstitution of the nutritional powder may be examined by measuring the percentage of the nutritional powder that is reconstituted when contacted by the liquid (e.g., does not remain in the pod following contact with the liquid, but is incorporated into the nutritional formula). The percent of reconstitution may be about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

The nutritional powder may be reconstituted in an amount of time from about 10 seconds to about 5 minutes. The reconstitution time is the time it takes for the 75% to 100% of the powder to be reconstituted to provide the nutritional formula, by the nutrient delivery system as described above. The reconstitution time is important in determining the flow characteristics of the formula through the nutrient delivery system. The reconstitution time is dependent on properties of the nutritional powder such as, powder particle size, porosity and shape. The reconstitution time may be determined by examining aliquots of the nutritional formula as it is produced by the nutrient delivery system (e.g., at intervals of time such as about every 5 seconds or about every 10 seconds), and calculating the total solids delivered over time using the interval samples. The reconstitution time may be about 10 seconds, 15 seconds, 20 seconds, 25 seconds, 30 seconds, 35 seconds, 40 seconds, 45 seconds, 50 seconds, 55 seconds, 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes.

Techniques used in the analysis of reconstitution, may vary in regards to temperatures and pressures used to remove the liquid. The liquid may be removed at temperatures of about 20° C., 22° C., 24° C., 26° C., 28° C., 30° C., 32° C., 34° C., 36° C., 38° C., 40° C., 42° C., 44° C., 46° C., 48° C., 50° C., 52° C., 54° C., 56° C., 58° C., 60° C., 62° C., 64° C., 66° C., 68° C., 70° C., 72° C., 74° C., 76° C., 78° C., 80° C., 82° C., 84° C., 86° C., 88° C., 90° C., 92° C., 94° C., 96° C., 98° C., 100° C., 102° C., 104° C., 106° C., 108° C., or 110° C. The pressure may be lowered by techniques known within the art, such as a vacuum pump. Pressures that may be used to remove the liquid in the analysis of reconstitution may be about 1 mbar, 10 mbar, 20 mbar, 40 mbar, 60 mbar, 80 mbar, 100 mbar, 120 mbar, 140 mbar, 160 mbar, 180 mbar, 200 mbar, 220 mbar, 240 mbar, 260 mbar, 280 mbar, 300 mbar, 320 mbar, 340 mbar, 360 mbar, 380 mbar, 400 mbar, 420 mbar, 440 mbar, 460 mbar, 480 mbar, 500 mbar, 520 mbar, 540 mbar, 560 mbar, 580 mbar, 600 mbar, 620 mbar, 640 mbar, 660 mbar, 680 mbar, 700 mbar, 720 mbar, 740 mbar, 760 mbar, 780 mbar, 800 mbar, 820 mbar, 840 mbar, 860 mbar, 880 mbar, 900 mbar, 920 mbar, 940 mbar, 960 mbar, 980 mbar, 1.0 bar, 1.5 bar, 2.0 bar, 2.5 bar, 3.0 bar, 3.5 bar, 4.0 bar, 4.5 bar, 5.0 bar, 5.5 bar, 6.0 bar, 6.5 bar, 7.0 bar, 7.5 bar, 8.0 bar, 8.5 bar, 9.0 bar, 10 bar, 10.5 bar, 11 bar, 11.5 bar, 12 bar, 12.5 bar, 13 bar, 13.5 bar, 14 bar, 14.5 bar or 15 bar.

(10) Color Scale Values

The nutritional powder may comprise a Hunter Lab “L” value between about 20 and about 100. The Hunter Lab “L” value is a measurement of the lightness of the formula. The lightness of the nutritional powder is dependent on, but not limited to, the wettability, emulsion stability, and emulsion homogeneity. The Hunter Lab “L” value of the nutritional powder can be measured by a spectrophotometer, which allows quantitative measurement of the reflection or transmission properties of the powder as a function of wavelength. The Hunter Lab “L” value of the nutritional powder may be about 20.00, 25.00, 30.00, 35.00, 40.00, 45.00, 50.00, 55.00, 60.00, 65.00, 70.00, 75.00, 80.00, 80.10, 80.15, 80.20, 80.25, 80.30, 80.35, 80.40, 80.45, 80.50, 80.55, 80.60, 80.65, 80.70, 80.75, 80.80, 80.85, 80.90, 80.95, 81.00, 81.10, 81.15, 81.20, 81.25, 81.30, 81.35, 81.40, 81.45, 81.50, 81.55, 81.60, 81.65, 81.70, 81.75, 81.80, 81.85, 81.90, 81.95, 82.00, 82.10, 82.15, 82.20, 82.25, 82.30, 82.35, 82.40, 82.45, 82.50, 82.55, 82.60, 82.65, 82.70, 82.75, 82.80, 82.85, 82.90, 82.95, 83.00, 83.10, 83.15, 83.20, 83.25, 83.30, 83.35, 840, 83.45, 83.50, 83.55, 83.60, 83.65, 83.70, 83.75, 83.80, 83.85, 83.90, 83.95, 84.00, 86.00, 88.00, 90.00, 95.00 or 100.00.

The nutritional powder may comprise a Hunter Lab “a” value between about −5.00 and about 1.00. The Hunter Lab “a” value is a measurement of the color-opponent dimension of a formula. The “a” value of the nutritional powder is dependent on, but not limited to, the wettability, emulsion stability, and emulsion homogeneity. The Hunter Lab “a” value of the nutritional powder can be measured by a spectrophotometer, which allows quantitative measurement of the reflection or transmission properties of the powder as a function of wavelength. The Hunter Lab “a” value of the nutritional powder may be about −5.00, −4.50, −4.00, −3.50, −3.00, −2.50, −2.00, −1.50, −1.00, −0.50, −0.10, −0.09, −0.08, −0.07, −0.06, −0.05, −0.04, −0.03, −0.02, −0.01, 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.22, 0.24, 0.26, 0.28, 0.3, 0.35, 0.40, 0.45, 0.50, 0.55, 0.60, 0.65, 0.70, 0.75, 0.80, 0.85, 0.90, or 1.00.

The nutritional powder may comprise a Hunter Lab “b” value between about 1 and about 30. The Hunter Lab “b” value is a measurement of the color-opponent dimension of a formula. The “b” value of the nutritional powder is dependent on, but not limited to, the wettability, emulsion stability, and emulsion homogeneity. The Hunter Lab “b” value of the nutritional powder can be measured by a spectrophotometer, which allows quantitative measurement of the reflection or transmission properties of the powder as a function of wavelength. The Hunter Lab “b” value of the nutritional powder may be about 1.00, 2.00, 3.00, 4.00, 5.00, 6.00, 7.00, 8.00, 9.00, 10.00, 11.00, 12.00, 13.00, 13.10, 13.20, 13.30, 13.31, 13.32, 13.33, 13.34, 13.35, 13.36, 13.37, 13.38, 13.39, 13.40, 13.41, 13.42, 13.43, 13.44, 13.45, 13.46, 13.47, 13.48, 13.49, 13.50, 13.51, 13.52, 13.53, 13.54, 13.55, 13.56, 13.57, 13.58, 13.59, 13.60, 13.61, 13.62, 13.63, 13.64, 13.65, 13.66, 13.67, 13.68, 13.69, 13.70, 13.71, 13.72, 13.73, 13.74, 13.75, 13.76, 13.77, 13.78, 13.79, 13.80, 13.81, 13.82, 13.83, 13.84, 13.85, 13.86, 13.87, 13.88, 13.89, 13.90, 13.91, 13.92, 13.93, 13.94, 13.95, 13.96, 13.97, 13.98, 13.99, 14.00, 15.00, 16.00, 17.00, 18.00, 19.00, 20.00, 25.00, or 30.00.

(11) Macronutrients

Nutritional powders (e.g., infant nutritional powders) according to the present disclosure may comprise one or more macronutrients selected from the group of fat, protein, carbohydrate, and mixtures thereof Generally, any source of fat, carbohydrate, or protein that is suitable for use in nutritional products is also suitable for use herein, provided that such macronutrients are also compatible with the essential elements of the nutritional powders, nutritional formulas and nutrient delivery systems as defined herein.

Although total concentrations or amounts of fat, protein, and carbohydrates may vary depending upon the nutritional needs of the subject, such concentrations or amounts most typically fall within one of the following embodied ranges, inclusive of any other essential fat, protein, and or carbohydrate ingredients as described herein.

Carbohydrate concentrations in the nutritional powders may typically range from about 5 wt % to about 70 wt %, including from about 7 wt % to about 60 wt %, including from about 10 wt % to about 55 wt %, by weight of the nutritional powders. Fat concentrations most typically range from about 0.5 wt % to about 35 wt %, including from about 0.75 wt % to about 30 wt %, including from about 1 wt % to about 28 wt %, and also including from about 2 wt % to about 5 wt %, by weight of the nutritional powders. Protein concentrations may range from about 1 wt % to about 85 wt %, from about 5 wt % to about 50 wt %, from about 7 wt % to about 32 wt %, or from about 8 wt % to about 30 wt %, by weight of the nutritional powders.

Additional ranges for carbohydrates, fats, and proteins, in those embodiments where the nutritional powder is formulated to provide an infant formula, based on percent of calories of the nutritional powder, are set forth in Table 1. Note: each numerical value in Table 1 is preceded by the term “about.”

TABLE 1

| | Embodiment | Embodiment | Embodiment | Embodiment |
|---|---|---|---|---|
| | A | B | C | D |
| Macronutrient | (% Calories) | (% Calories) | (% Calories) | (% Calories) |
| Carbohydrate | 1-85 | 30-75 | 35-65 | 30-50 |
| Fat | 5-70 | 20-60 | 25-50 | 40-60 |
| Protein | 2-75 | 5-50 | 7-40 | 5-15 |

Additional ranges for carbohydrates, fats, and proteins, in those embodiments where the nutritional powder is formulated to provide an adult formula, based on percent of calories of the nutritional powder, are set forth in Table 2. Note: each numerical value in Table 2 is preceded by the term “about.”

TABLE 2

| | Embodiment A | Embodiment B | Embodiment C |
|---|---|---|---|
| Macronutrient | (% Calories) | (% Calories) | (% Calories) |
| Protein | 1-98 | 5-80 | 15-55 |
| Carbohydrate | 1-98 | 0-75 | 20-50 |
| Fat | 1-98 | 20-70 | 25-40 |

For example, in some embodiments the nutritional powder may include: about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69% or 70% carbohydrate as a percentage of total calories; about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% or 80% fat as a percentage of total calories; and about 5%, 6%, 7%, 8%, 9%, 10%, 11%, 12%, 13%, 14% or 15% protein as a percentage of total calories.

In some embodiments, the nutritional powder includes one or more of a protein source, a carbohydrate source, or a fat source. In those embodiments where the nutritional powder includes a protein source, the protein source can include one or more of whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, soy protein concentrates, soy protein isolates, soy protein hydrolysates, pea protein concentrates, pea protein isolates, pea protein hydrolysates, collagen proteins, potato proteins, rice proteins, fungal proteins, proteins expressed by microorganisms, and combinations thereof.

In those embodiments where the nutritional powder includes a carbohydrate source, the carbohydrate source can include one or more of maltodextrin; hydrolyzed or modified starch or cornstarch; glucose polymers; corn syrup; corn syrup solids; rice-derived carbohydrates; high fructose corn syrup; honey; sugar alcohols, such as maltitol, erythritol, sorbitol, glycerine; sucrose; glucose; fructose; lactose; isomaltulose, sucromalt, pullulan, potato starch, and other slowly-digested carbohydrates; oligosaccharides such as fructo-oligosaccharides; dietary fibers including, but not limited to, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low and high methoxy pectin, cereal beta-glucans, carrageenan and psyllium, soluble dietary fibers such as digestion resistant maltodextrins, resistant modified food starches, other resistant starches; soluble and insoluble fibers derived from fruits or vegetables; and combinations thereof

In those embodiments where the nutritional powder includes a fat source, the fat source can include one or more of coconut oil, fractionated coconut oil, soy oil, corn oil, olive oil, safflower oil, high oleic safflower oil, medium chain triglyceride oil, high gamma linolenic safflower oil, sunflower oil, high oleic sunflower oil, palm oil, palm kernel oil, palm olein, canola oil, marine oils, algal oils, cottonseed oils, interesterified oils, transesterified oils, and combinations thereof

In those embodiments where the nutritional powder includes a fat source, the fat source can include a fatty acid. The fatty acid may include palmitic acid, myristic acid, stearic acid, linoleic acid, alpha-linoleic acid, and combinations thereof The nutritional powder may comprise a fatty acid, such as palmitic acid, up to about 5% by weight of the total fat content, including about 0.1% to about 5%, about 0.1% to about 4%, about 0.1% to about 3%, about 0.1% to about 2%, about 0.1% to about 1.0%, about 1% to about 5%, about 1% to about 4%, about 1% to about 3%, about 1% to about 2%, about 2% to about 5%, about 0.2% to about 1.0%, about 0.3% to about 1.0%, about 0.4% to about 1.0%, about 0.5% to about 1.0%, about 0.6% to about 1.0%, about 0.7% to about 1.0%, about 0.8% to about 1.0%, about 0.9% to about 1.0%, about 0.2% to about 1.0%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 0.9%, about 0.3% to about 0.8%, about 0.3% to about 0.7%, about 0.3% to about 0.7%, about 0.3% to about 0.6%, about 0.3% to about 0.5%, about 0.3% to about 0.4%, about 0.4% to about 0.9%,about 0.4% to about 0.8%, about 0.4% to about 0.7%, about 0.4% to about 0.6%, about 0.4% to about 0.5%, about 0.5% to about 0.9%, about 0.5% to about 0.8%, about 0.5% to about 0.7%, about 0.5% to about 0.6%, about 0.6% to about 0.9%, about 0.6% to about 0.8%, about 0.6% to about 0.7%, about 0.7% to about 0.9%, about 0.7% to about 0.8%, or about 0.8% to about 0.9%, by weight of the total fat content. The total fat content may comprise, by weight, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.1%, at least about 3.2%, at least about 3.3%, at least about 3.4%, at least about 3.5%, at least about 3.6%, at least about 3.7%, at least about 3.8%, at least about 3.9%, at least about 4%, at least about 4.1%, at least about 4.2%, at least about 4.3%, at least about 4.4%, at least about 4.5%, at least about 4.6%, at least about 4.7%, at least about 4.8%, at least about 4.9%, or at least about 5% of a fatty acid, such as palmitic acid. The total fat content may comprise, by weight, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5% of a fatty acid, such as palmitic acid.

In some embodiments, the nutritional powders include a protein component that consists of only intact or partially hydrolyzed protein; that is, the protein component is substantially free of any protein that has a degree of hydrolysis of 25% or more. In this context, the term “partially hydrolyzed protein” refers to proteins having a degree of hydrolysis of less than 25%, including less than 20%, including less than 15%, including less than 10%, and including proteins having a degree of hydrolysis of less than 5%. The degree of hydrolysis is the extent to which peptide bonds are broken by a hydrolysis chemical reaction. To quantify the partially hydrolyzed protein component of these embodiments, the degree of protein hydrolysis is determined by quantifying the amino nitrogen to total nitrogen ratio (AN/TN) of the protein component of the selected nutritional powder. The amino nitrogen component is quantified by USP titration methods for determining amino nitrogen content, while the total nitrogen component is determined by the Tecator® Kjeldahl method. These analytical methods are well known.

(12) Human Milk Oligosaccharides

The nutritional powders of the present disclosure include at least one HMO, and in many embodiments, a combination of two or more HMOs. Oligosaccharides are one of the main components of human breast milk, which contains, on average, 10 grams per liter of neutral oligosaccharides and 1 gram per liter of acidic oligosaccharides. The composition of human milk oligosaccharides is very complex and more than 200 different oligosaccharide-like structures are known.

The HMOs may be included in the nutritional powders and formulas alone, or in some embodiments, in combination with other immune enhancing factors (e.g., LCPUFAs, antioxidants, nucleotides, etc.) as described herein. The HMO or HMOs may be isolated or enriched from milk(s) secreted by mammals including, but not limited to: human, bovine, ovine, porcine, or caprine species. The HMOs may also be produced via microbial fermentation, enzymatic processes, chemical synthesis, or combinations thereof

Suitable HMOs for use in the nutritional powders may include acidic oligosaccharides, neutral oligosaccharides, n-acetylglucosylated oligosaccharides, and HMO precursors. Specific non-limiting examples of HMOs that may be included individually or in combination in the nutritional powders of the present disclosure include: sialic acid (i.e., free sialic acid, lipid-bound sialic acid, protein-bound sialic acid); D-glucose (Glc); D-galactose (Gal); N-acetylglucosamine (GlcNAc); L-fucose (L-Fuc); D-fucose (D-Fuc); fucosyl oligosaccharides (i.e., Lacto-N-fucopentaose I; Lacto-N-fucopentaose II; 2′-Fucosyllactose; 3′-Fucosyllactose; Lacto-N-fucopentaose III; Lacto-N-difucohexaose I; and Lactodifucotetraose); non-fucosylated, non-sialylated oligosaccharides (i.e., Lacto-N-tetraose and Lacto-N-neotetraose); sialyl oligosaccharides (i.e., 3′-Sialyl-3-fucosyllactose; Disialomonofucosyllacto-N-neohexaose; Monofucosylmonosialyllacto-N-octaose (sialyl Lea); Sialyllacto-N-fucohexaose II; Disialyllacto-N-fucopentaose II; Monofucosyldisialyllacto-N-tetraose); and sialyl fucosyl oligosaccharides (i.e., 2′-Sialyllactose; 2-Sialyllactosamine; 3′-Sialyllactose; 3′-Sialyllactosamine; 6′-Sialyllactose; 6′-Sialyllactosamine; Sialyllacto-N-neotetraose c; Monosialyllacto-N-hexaose; Disialyllacto-N-hexaose I; Monosialyllacto-N-neohexaose I; Monosialyllacto-N-neohexaose II; Disialyllacto-N-neohexaose; Disialyllacto-N-tetraose; Disialyllacto-N-hexaose II; Sialyllacto-N-tetraose a; Disialyllacto-N-hexaose I; and Sialyllacto-N-tetraose b). Also useful are variants in which the glucose (Glc at the reducing end is replaced by N-acetylglucosamine (e.g., 2′-fucosyl-N-acetylglucosamine (2′-FLNac) is such a variant to 2′-fucosyllactose). These HMOs are described more fully in U.S. Patent Application No. 2009/0098240, which is herein incorporated by reference in its entirety. Other suitable examples of HMOs that may be included in the nutritional powders of the present disclosure include lacto-N-fucopentaose V, lacto-N-hexaose, para-lacto-N-hexaose, lacto-N-neohexaose, para-lacto-N-neohexaose, monofucosyllacto-N-hexaose II, isomeric fucosylated lacto-N-hexaose (1), isomeric fucosylated lacto-N-hexaose (3), isomeric fucosylated lacto-N-hexaose (2), difucosyl-para-lacto-N-neohexaose, difucosyl-para-lacto-N-hexaose, difucosyllacto-N-hexaose, lacto-N-neoocataose, para-lacto-N-octanose, iso-lacto-N-octaose, lacto-N-octaose, monofucosyllacto-neoocataose, monofucosyllacto-N-ocataose, difucosyllacto-N-octaose I, difucosyllacto-N-octaose II, difucosyllacto-N-neoocataose II, difucosyllacto-N-neoocataose I, lacto-N-decaose, trifucosyllacto-N-neooctaose, trifucosyllacto-N-octaose, trifucosyl-iso-lacto-N-octaose, lacto-N-difuco-hexaose II, sialyl-lacto-N-tetraose a, sialyl-lacto-N-tetraose b, sialyl-lacto-N-tetraose c, sialyl-fucosyl-lacto-N-tetraose I, sialyl-fucosyl-lacto-N-tetraose II, and disialyl-lacto-N-tetraose, and combinations thereof Particularly suitable nutritional powders include at least one of the following HMOs or HMO precursors: sialic acid (SA); 3′-Sialyllactose (3′SL); 6′-Sialyllactose (6′SL); 2′-Fucosyllactose (2′FL); 3′-Fucosyllactose (3′FL); Lacto-N-tetraose (LNT) and Lacto-N-neotetraose (LNnT), and in particular, combinations of 6′SL and 3′SL; combinations of 3′FL and SA; combinations of 2′FL and 3′FL; combinations of 2′FL, 3′SL, and 6′SL; combinations of 3′SL, 3′FL, and LNnT; and combinations of 6′SL, 2′FL, and LNnT.

Other exemplary combinations include: SA, 3′SL, 6′SL, 3′FL, 2′FL, and LNnT; 3′SL, 6′SL, 3′FL, 2′FL, and LNnT; SA, 6′SL, 3′FL, 2′FL, and LNnT; SA, 3′SL, 3′FL, 2′FL, and LNnT; SA, 3′SL, 6′SL, 2′FL, and LNnT; SA, 3′SL, 6′SL, 3′FL, and LNnT; SA, 3′SL, 6′SL, 3′FL, and 2′FL; SA and 3′SL; SA and 6′SL; SA and 2′FL; SA and LNnT; SA, 3′SL, and 6′SL; SA, 3′SL and 3′FL; SA, 3′SL and 2′FL; SA, 3′SL and LNnT; SA, 6′SL and 3′FL; SA, 6′SL, and 2′FL; SA, 6′SL, and LNnT; SA, 3′FL, and 2′FL; SA, 3′FL, and LNnT; SA, 2′FL, and LNnT; SA, 3′SL, 6′SL, and 3′FL; SA, 3′SL, 6′SL and 2′FL; SA, 3′SL, 6′SL, and LNnT; SA, 3′SL, 3′FL, and 2′FL; SA, 3′SL, 3′FL, and LNnT; SA, 3′SL, 2′FL, and LNnT; SA, 6′SL, 3′FL, and 2′FL; SA, 6′SL, 2′FL, and LNnT; SA, 6′SL, 3′FL, and LNnT; SA, 3′FL, 2′FL, and LNnT; SA, 6′SL, 2′FL, and LNnT; SA, 3′SL, 3′FL, 2′FL, and LNnT; SA, 6′SL, 3′FL, 2′FL, and LNnT; SA, 3′SL, 6′SL, 3′FL, and LNnT; SA, 3′SL, 3′FL, 2′FL, and LNnT; SA, 3′SL, 6′SL, 2′FL, and LNnT; 3′SL, 6′SL, 3′FL, and 2′FL; 3′SL, 6′SL, 2′FL, and LNnT; 3′SL, 3′FL, 2′FL, and LNnT; 3′SL, 6′SL, 3′FL, and LNnT; 3′SL, 6′SL, and 3′FL; 3′SL, 3′FL, and 2′FL; 3′SL, 2′FL, and LNnT; 3′SL, 6′SL, and 2′FL; 3′SL, 6′SL, and LNnT; 3′SL and 3′FL; 3′SL and 2′FL; 3′SL and LNnT; 6′SL and 3′FL; 6′SL and 2′FL; 6′SL and LNnT; 6′SL, 3′FL, and LNnT; 6′SL, 3′FL, 2′FL, and LNnT; 3′FL, 2′FL, and LNnT; 3′FL and LNnT; and 2′FL and LNnT.

In one specific embodiment of a nutritional powder, the total concentration of HMOs in the nutritional powder is from about 0.0005% to about 5%, including from 0.0005% to about 5%, or about 0.01% to about 1% (by weight of the nutritional powder). For example, the nutritional powder may have a total concentration of HMOs of about 0.0005%, 0.001%, 0.002%, 0.003%, 0.004%, 0.005%, 0.006%, 0.007%, 0.008%, 0.009%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 6%, 7%, 8%, 9%, 10%, 11%, 12%, 13%, 14%, 15%, 16%, 17%, 18%, 19%, or 20% by weight of the nutritional powder.

In one specific embodiment of the present disclosure, a nutritional powder includes 2′FL. The 2′FL may be the only HMO included in the nutritional composition, or other additional HMOs may also be included in the nutritional powder (e.g., the 2′FL may be combined with 3′SL and/or 6′SL in some specific embodiments).

In one specific embodiment, the nutritional powder includes 6′SL, alone or in combination with other HMOs. In one embodiment, when the nutritional powder includes 6′SL, the total amount of HMOs in the nutritional powder includes at least about 88% (by total weight HMOs) 6′SL, including from about 88% (by total weight HMOs) to about 96% (by total weight HMOs), including from about 88% (by total weight HMOs) to about 100% (by total weight HMOs), and including about 100% (by total weight HMOs) 6′SL.

In another embodiment, the nutritional powder includes 3′SL, alone or in combination with other HMOs. In one embodiment, when the nutritional powder includes 3′SL, the total amount of HMOs in the nutritional powder includes at least about 85% (by total weight HMOs) 3′SL, including from about 85% (by total weight HMOs) to about 88% (by total weight HMOs), including from about 88% (by total weight HMOs) to about 100% (by total weight HMOs), and including about 100% (by total weight HMOs) 3′SL.

In one specific embodiment, the nutritional powder includes LNnT, alone or in combination with other HMOs.

In some embodiments, the HMOs are used in combination to provide the desired immune enhancing effect. For example, in one embodiment, the nutritional powder includes 6′SL in combination with 3′SL. In another embodiment, the nutritional powder includes 6′SL in combination with 3′SL. In another embodiment, the nutritional powder includes 3′SL and 6′SL in a weight ratio of from about 1:20 to about 20:1, including from about 1:10 to about 10:1, and including from about 1:2 to about 2:1.

In another specific embodiment, the nutritional powder includes 3′FL, alone or in combination with other HMOs.

In one specific embodiment, the nutritional composition includes 3′FL in combination with SA.

In another embodiment, the nutritional composition includes 2′FL, alone or in combination with other HMOs.

In one specific embodiment, the nutritional composition includes 2′FL in combination with 3′FL.

In yet another embodiment, the nutritional composition includes a combination of 6′ SL, 2′FL, and LNnT.

(13) Additional Prebiotic Oligosaccharides

The nutritional powders of the present disclosure may, in addition to the HMOs described above, comprise an additional source or sources of prebiotic oligosaccharides (the total amount of oligosaccharides being referred to herein as an “oligosaccharide blend” of the nutritional composition). Suitable additional sources of prebiotic oligosaccharides for use in the nutritional powders include any prebiotic oligosaccharide that is suitable for use in an oral nutritional composition and is compatible with the essential elements and features of such compositions. In some embodiments, the nutritional powder includes a combination of one or more HMOs and one or more additional prebiotic oligosaccharides such that the powder (and ultimate nutritional formula) provides a synergistic benefit to the end user, such as a synergistic benefit in improving feeding intolerance in infants.

In some embodiments, the combinations of HMO or HMOs with the additional prebiotic oligosaccharides to provide the synergistic effect include HMOs and additional prebiotic oligosaccharides that ferment at a rapid rate (“rapidly-fermenting oligosaccharides”), oligosaccharides that ferment at a moderate rate (“medium-fermenting oligosaccharides”), and/or oligosaccharides that ferment at a slow rate (“slowly-fermenting oligosaccharides”). Some preferred embodiments provide a nutritional powder that includes at least one HMO in combination with a rapidly-fermenting oligosaccharide, a medium-fermenting oligosaccharide, and/or a slowly-fermenting oligosaccharide.

Non-limiting examples of suitable additional prebiotic oligosaccharides for use in the nutritional powders described herein include prebiotic oligosaccharides that have a degree of polymerization (DP) of at least 2 monose units, which are not or only partially digested in the intestine by the action of acids or digestive enzymes present in the human upper digestive tract (small intestine and stomach), but which are fermentable by the human intestinal flora. The term “monose units” refers to units having a closed ring structure, preferably hexose, e.g., the pyranose or furanose forms. Particularly preferred oligosaccharides for use in combination with the HMO or HMOs in the nutritional powders of the present disclosure include galactooligosaccharides (GOS), fructooligosaccharides (FOS), short chain fructooligosaccharides, inulin, oligofructose, polydextrose (PDX), pectin hydrolysate, and gum fiber. In one specific embodiment, the gum fiber is gum arabic.

Typically, when used as an oligosaccharide blend, the nutritional powders, in addition to the HMO or HMOs, include at least one rapidly-fermented oligosaccharide, at least one medium-fermented oligosaccharide, and, optionally, at least one slowly-fermented oligosaccharide to provide a nutritional powder that is tolerated well infants (i.e., reduced gassiness and/or stool frequency). Rapidly-fermented oligosaccharides generally have a fermentation rate of greater than 4,000 μg/g of dry matter/hour; medium-fermented oligosaccharides generally have a fermentation rate of from 1,500 μg/g of dry matter/hour to 4,000 μg/g of dry matter/hour; and slowly-fermented oligosaccharides generally have a fermentation rate of less than 1,500 μg/g of dry matter/hour.

By way of specific example, rapidly-fermented oligosaccharides include FOS, GOS (about 9,304 μg/g of dry matter/hour), LNnT (about 4,488 μg/g of dry matter/hour), 2′FL (about 4,872 μg/g of dry matter/hour), and combinations thereof. Medium-fermented oligosaccharides include 6′SL (about 1,809 μg/g of dry matter/hour), 3′SL, 2′FL, 3′FL, LNnT and combinations thereof. Slowly-fermented oligosaccharides include longer chain carbohydrates such as inulin (about 1,435 μg/g of dry matter/hour), gum fibers (e.g., gum arabic (about 785 μg/g of dry matter/hour)), and combinations thereof.

In one specific embodiment, the nutritional powder includes an oligosaccharide blend including LNnT, 6′SL and inulin.

In another specific embodiment, the nutritional formula includes an oligosaccharide blend including 2′FL, 6′SL and inulin.

Other exemplary combinations include: FOS, GOS, 2′FL, LNnT, 3′SL, and 6′SL; FOS, GOS, 2′FL, 3′SL, and 6′SL; FOS, GOS, LNnT, 3′SL, and 6′SL; FOS, 2′FL, LNnT, 3′SL, and 6′SL; GOS, 2′FL, LNnT, 3′SL, and 6′SL; FOS, GOS, 3′SL, and 6′SL; FOS, 2′FL, 3′SL, and 6′SL; FOS, LNnT, 3′SL, and 6′SL; GOS, 2′FL, 3′SL, and 6′SL; GOS, LNnT, 3′SL, and 6′SL; 2′FL, LNnT, 3′SL, and 6′SL; FOS, 3′SL, and 6′SL; GOS, 3′SL, and 6′SL; 2′FL, 3′SL, and 6′SL; LNnT, 3′SL, and 6′SL; FOS, GOS, 2′FL, LNnT, and 3′SL; FOS, GOS, 2′FL, and 3′SL; FOS, GOS, LNnT, and 3′SL; FOS, 2′FL, LNnT, and 3′SL; GOS, 2′FL, LNnT, and 3′SL; FOS, GOS, and 3′SL; FOS, 2′FL, and 3′SL; FOS, LNnT, and 3′SL; GOS, 2′FL, and 3′SL; GOS, LNnT, and 3′SL; 2′FL, LNnT, and 3′SL; FOS and 3′SL; GOS and 3′SL; 2′FL and 3′SL; LNnT and 3′SL; FOS, GOS, 2′FL, LNnT, and 6′SL; FOS, GOS, 2′FL, and 6′SL; FOS, GOS, LNnT, and 6′SL; FOS, 2′FL, LNnT, and 6′SL; GOS, 2′FL, LNnT, and 6′SL; FOS, GOS, and 6′SL; FOS, 2′FL, and 6′SL; FOS, LNnT, and 6′SL; GOS, 2′FL, and 6′SL; GOS, LNnT, and 6′SL; 2′FL, LNnT, and 6′SL; FOS and 6′SL; GOS and 6′SL; 2′FL and 6′SL; and LNnT and 6′SL.

Further exemplary combinations include: FOS, GOS, 2′FL, LNnT, 3′SL, 6′SL, inulin, a gum, and polydextrose; FOS, GOS, 2′FL, 3′SL, 6′SL, inulin, a gum, and polydextrose; FOS, GOS, LNnT, 3′SL, 6′SL, inulin, a gum, and polydextrose; FOS, 2′FL, LNnT, 3′SL, 6′SL, inulin, a gum, and polydextrose; GOS, 2′FL, LNnT, 3′SL, 6′SL, inulin, a gum, and polydextrose; FOS, GOS, 3′SL, 6′SL, inulin, a gum, and polydextrose; FOS, 2′FL, 3′SL, 6′SL, inulin, a gum, and polydextrose; FOS, LNnT, 3′SL, 6′SL, inulin, a gum, and polydextrose; GOS, 2′FL, 3′SL, 6′SL, inulin, a gum, and polydextrose; GOS, LNnT, 3′SL, 6′SL, inulin, a gum, and polydextrose; 2′FL, LNnT, 3′SL, 6′SL, inulin, a gum, and polydextrose; FOS, 3′SL, 6′SL, inulin, a gum, and polydextrose; GOS, 3′SL, 6′SL, inulin, a gum, and polydextrose; 2′FL, 3′SL, 6′SL, inulin, a gum, and polydextrose; LNnT, 3′SL, 6′SL, inulin, a gum, and polydextrose; FOS, GOS, 2′FL, LNnT, 3′SL, inulin, a gum, and polydextrose; FOS, GOS, 2′FL, 3′SL, inulin, a gum, and polydextrose; FOS, GOS, LNnT, 3′SL, inulin, a gum, and polydextrose; FOS, 2′FL, LNnT, 3′SL, inulin, a gum, and polydextrose; GOS, 2′FL, LNnT, 3′SL, inulin, a gum, and polydextrose; FOS, GOS, 3′SL, inulin, a gum, and polydextrose; FOS, 2′FL, 3′SL, inulin, a gum, and polydextrose; FOS, LNnT, 3′SL, inulin, a gum, and polydextrose; GOS, 2′FL, 3′SL, inulin, a gum, and polydextrose; GOS, LNnT, 3′SL, inulin, a gum, and polydextrose; 2′FL, LNnT, 3′SL, inulin, a gum, and polydextrose; FOS, 3′SL, inulin, a gum, and polydextrose; GOS, 3′SL, inulin, a gum, and polydextrose; 2′FL, 3′SL, inulin, a gum, and polydextrose; LNnT, 3′SL, inulin, a gum, and polydextrose; FOS, GOS, 2′FL, LNnT, 6′SL, inulin, a gum, and polydextrose; FOS, GOS, 2′FL, 6′SL, inulin, a gum, and polydextrose; FOS, GOS, LNnT, 6′SL, inulin, a gum, and polydextrose; FOS, 2′FL, LNnT, 6′SL, inulin, a gum, and polydextrose; GOS, 2′FL, LNnT, 6′SL, inulin, a gum, and polydextrose; FOS, GOS, 6′SL, inulin, a gum, and polydextrose; FOS, 2′FL, 6′SL, inulin, a gum, and polydextrose; FOS, LNnT, 6′SL, inulin, a gum, and polydextrose; GOS, 2′FL, 6′SL, inulin, a gum, and polydextrose; GOS, LNnT, 6′SL, inulin, a gum, and polydextrose; 2′FL, LNnT, 6′SL, inulin, a gum, and polydextrose; FOS, 6′SL, inulin, a gum, and polydextrose; GOS, 6′SL, inulin, a gum, and polydextrose; 2′FL, 6′SL, inulin, a gum, and polydextrose; LNnT, 6′SL, inulin, a gum, and polydextrose; FOS, GOS, 2′FL, LNnT, 3′SL, 6′SL, inulin, and a gum; FOS, GOS, 2′FL, 3′SL, 6′SL, inulin, and a gum; FOS, GOS, LNnT, 3′SL, 6′SL, inulin, and a gum; FOS, 2′FL, LNnT, 3′SL, 6′SL, inulin, and a gum; GOS, 2′FL, LNnT, 3′SL, 6′SL, inulin, and a gum; FOS, GOS, 3′SL, 6′SL, inulin, and a gum; FOS, 2′FL, 3′SL, 6′SL, inulin, and a gum; FOS, LNnT, 3′SL, 6′SL, inulin, and a gum; GOS, 2′FL, 3′SL, 6′SL, inulin, and a gum; GOS, LNnT, 3′SL, 6′SL, inulin, and a gum; 2′FL, LNnT, 3′SL, 6′SL, inulin, and a gum; FOS, 3′SL, 6′SL, inulin, and a gum; GOS, 3′SL, 6′SL, inulin, and a gum; 2′FL, 3′SL, 6′SL, inulin, and a gum; LNnT, 3′SL, 6′SL, inulin, and a gum; FOS, GOS, 2′FL, LNnT, 3′SL, inulin, and a gum; FOS, GOS, 2′FL, 3′SL, inulin, and a gum; FOS, GOS, LNnT, 3′SL, inulin, and a gum; FOS, 2′FL, LNnT, 3′SL, inulin, and a gum; GOS, 2′FL, LNnT, 3′SL, inulin, and a gum; FOS, GOS, 3′SL, inulin, and a gum; FOS, 2′FL, 3′SL, inulin, and a gum; FOS, LNnT, 3′SL, inulin, and a gum; GOS, 2′FL, 3′SL, inulin, and a gum; GOS, LNnT, 3′SL, inulin, and a gum; 2′FL, LNnT, 3′SL, inulin, and a gum; FOS, 3′SL, inulin, and a gum; GOS, 3′SL, inulin, and a gum; 2′FL, 3′SL, inulin, and a gum; LNnT, 3′SL, inulin, and a gum; FOS, GOS, 2′FL, LNnT, 6′SL, inulin, and a gum; FOS, GOS, 2′FL, 6′SL, inulin, and a gum; FOS, GOS, LNnT, 6′SL, inulin, and a gum; FOS, 2′FL, LNnT, 6′SL, inulin, and a gum; GOS, 2′FL, LNnT, 6′SL, inulin, and a gum; FOS, GOS, 6′SL, inulin, and a gum; FOS, 2′FL, 6′SL, inulin, and a gum; FOS, LNnT, 6′SL, inulin, and a gum; GOS, 2′FL, 6′SL, inulin, and a gum; GOS, LNnT, 6′SL, inulin, and a gum; 2′FL, LNnT, 6′SL, inulin, and a gum; FOS, 6′SL, inulin, and a gum; GOS, 6′SL, inulin, and a gum; 2′FL, 6′SL, inulin, and a gum; LNnT, 6′SL, inulin, and a gum; FOS, GOS, 2′FL, LNnT, 3′SL, 6′SL, inulin, and polydextrose; FOS, GOS, 2′FL, 3′SL, 6′SL, inulin, and polydextrose; FOS, GOS, LNnT, 3′SL, 6′SL, inulin, and polydextrose; FOS, 2′FL, LNnT, 3′SL, 6′SL, inulin, and polydextrose; GOS, 2′FL, LNnT, 3′SL, 6′SL, inulin, and polydextrose; FOS, GOS, 3′SL, 6′SL, inulin, and polydextrose; FOS, 2′FL, 3′SL, 6′SL, inulin, and polydextrose; FOS, LNnT, 3′SL, 6′SL, inulin, and polydextrose; GOS, 2′FL, 3′SL, 6′SL, inulin, and polydextrose; GOS, LNnT, 3′SL, 6′SL, inulin, and polydextrose; 2′FL, LNnT, 3′SL, 6′SL, inulin, and polydextrose; FOS, 3′SL, 6′SL, inulin, and polydextrose; GOS, 3′SL, 6′SL, inulin, and polydextrose; 2′FL, 3′SL, 6′SL, inulin, and polydextrose; LNnT, 3′SL, 6′SL, inulin, and polydextrose; FOS, GOS, 2′FL, LNnT, 3′SL, inulin, and polydextrose; FOS, GOS, 2′FL, 3′SL, inulin, and polydextrose; FOS, GOS, LNnT, 3′SL, inulin, and polydextrose; FOS, 2′FL, LNnT, 3′SL, inulin, and polydextrose; GOS, 2′FL, LNnT, 3′SL, inulin, and polydextrose; FOS, GOS, 3′SL, inulin, and polydextrose; FOS, 2′FL, 3′SL, inulin, and polydextrose; FOS, LNnT, 3′SL, inulin, and polydextrose; GOS, 2′FL, 3′SL, inulin, and polydextrose; GOS, LNnT, 3′SL, inulin, and polydextrose; 2′FL, LNnT, 3′SL, inulin, and polydextrose; FOS, 3′SL, inulin, and polydextrose; GOS, 3′SL, inulin, and polydextrose; 2′FL, 3′SL, inulin, and polydextrose; LNnT, 3′SL, inulin, and polydextrose; FOS, GOS, 2′FL, LNnT, 6′SL, inulin, and polydextrose; FOS, GOS, 2′FL, 6′SL, inulin, and polydextrose; FOS, GOS, LNnT, 6′SL, inulin, and polydextrose; FOS, 2′FL, LNnT, 6′SL, inulin, and polydextrose; GOS, 2′FL, LNnT, 6′SL, inulin, and polydextrose; FOS, GOS, 6′SL, inulin, and polydextrose; FOS, 2′FL, 6′SL, inulin, and polydextrose; FOS, LNnT, 6′SL, inulin, and polydextrose; GOS, 2′FL, 6′SL, inulin, and polydextrose; GOS, LNnT, 6′SL, inulin, and polydextrose; 2′FL, LNnT, 6′SL, inulin, and polydextrose; FOS, 6′SL, inulin, and polydextrose; GOS, 6′SL, inulin, and polydextrose; 2′FL, 6′SL, inulin, and polydextrose; LNnT, 6′SL, inulin, and polydextrose; FOS, GOS, 2′FL, LNnT, 3′SL, 6′SL, a gum, and polydextrose; FOS, GOS, 2′FL, 3′SL, 6′SL, a gum, and polydextrose; FOS, GOS, LNnT, 3′SL, 6′SL, a gum, and polydextrose; FOS, 2′FL, LNnT, 3′SL, 6′SL, a gum, and polydextrose; GOS, 2′FL, LNnT, 3′SL, 6′SL, a gum, and polydextrose; FOS, GOS, 3′SL, 6′SL, a gum, and polydextrose; FOS, 2′FL, 3′SL, 6′SL, a gum, and polydextrose; FOS, LNnT, 3′SL, 6′SL, a gum, and polydextrose; GOS, 2′FL, 3′SL, 6′SL, a gum, and polydextrose; GOS, LNnT, 3′SL, 6′SL, a gum, and polydextrose; 2′FL, LNnT, 3′SL, 6′SL, a gum, and polydextrose; FOS, 3′SL, 6′SL, a gum, and polydextrose; GOS, 3′SL, 6′SL, a gum, and polydextrose; 2′FL, 3′SL, 6′SL, a gum, and polydextrose; LNnT, 3′SL, 6′SL, a gum, and polydextrose; FOS, GOS, 2′FL, LNnT, 3′SL, a gum, and polydextrose; FOS, GOS, 2′FL, 3′SL, a gum, and polydextrose; FOS, GOS, LNnT, 3′SL, a gum, and polydextrose; FOS, 2′FL, LNnT, 3′SL, a gum, and polydextrose; GOS, 2′FL, LNnT, 3′SL, a gum, and polydextrose; FOS, GOS, 3′SL, a gum, and polydextrose; FOS, 2′FL, 3′SL, a gum, and polydextrose; FOS, LNnT, 3′SL, a gum, and polydextrose; GOS, 2′FL, 3′SL, a gum, and polydextrose; GOS, LNnT, 3′SL, a gum, and polydextrose; 2′FL, LNnT, 3′SL, a gum, and polydextrose; FOS, 3′SL, a gum, and polydextrose; GOS, 3′SL, a gum, and polydextrose; 2′FL, 3′SL, a gum, and polydextrose; LNnT, 3′SL, a gum, and polydextrose; FOS, GOS, 2′FL, LNnT, 6′SL, a gum, and polydextrose; FOS, GOS, 2′FL, 6′SL, a gum, and polydextrose; FOS, GOS, LNnT, 6′SL, a gum, and polydextrose; FOS, 2′FL, LNnT, 6′SL, a gum, and polydextrose; GOS, 2′FL, LNnT, 6′SL, a gum, and polydextrose; FOS, GOS, 6′SL, a gum, and polydextrose; FOS, 2′FL, 6′SL, a gum, and polydextrose; FOS, LNnT, 6′SL, a gum, and polydextrose; GOS, 2′FL, 6′SL, a gum, and polydextrose; GOS, LNnT, 6′SL, a gum, and polydextrose; 2′FL, LNnT, 6′SL, a gum, and polydextrose; FOS, 6′SL, a gum, and polydextrose; GOS, 6′SL, a gum, and polydextrose; 2′FL, 6′SL, a gum, and polydextrose; LNnT, 6′SL, a gum, and polydextrose; FOS, GOS, 2′FL, LNnT, 3′SL, 6′SL, and inulin; FOS, GOS, 2′FL, 3′SL, 6′SL, and inulin; FOS, GOS, LNnT, 3′SL, 6′SL, and inulin; FOS, 2′FL, LNnT, 3′SL, 6′SL, and inulin; GOS, 2′FL, LNnT, 3′SL, 6′SL, and inulin; FOS, GOS, 3′SL, 6′SL, and inulin; FOS, 2′FL, 3′SL, 6′SL, and inulin; FOS, LNnT, 3′SL, 6′SL, and inulin; GOS, 2′FL, 3′SL, 6′SL, and inulin; GOS, LNnT, 3′SL, 6′SL, and inulin; 2′FL, LNnT, 3′SL, 6′SL, and inulin; FOS, 3′SL, 6′SL, and inulin; GOS, 3′SL, 6′SL, and inulin; 2′FL, 3′SL, 6′SL, and inulin; LNnT, 3′SL, 6′SL, and inulin; FOS, GOS, 2′FL, LNnT, 3′SL, and inulin; FOS, GOS, 2′FL, 3′SL, and inulin; FOS, GOS, LNnT, 3′SL, and inulin; FOS, 2′FL, LNnT, 3′SL, and inulin; GOS, 2′FL, LNnT, 3′SL, and inulin; FOS, GOS, 3′SL, and inulin; FOS, 2′FL, 3′SL, and inulin; FOS, LNnT, 3′SL, and inulin; GOS, 2′FL, 3′SL, and inulin; GOS, LNnT, 3′SL, and inulin; 2′FL, LNnT, 3′SL, and inulin; FOS, 3′SL, and inulin; GOS, 3′SL, and inulin; 2′FL, 3′SL, and inulin; LNnT, 3′SL, and inulin; FOS, GOS, 2′FL, LNnT, 6′SL, and inulin; FOS, GOS, 2′FL, 6′SL, and inulin; FOS, GOS, LNnT, 6′SL, and inulin; FOS, 2′FL, LNnT, 6′SL, and inulin; GOS, 2′FL, LNnT, 6′SL, and inulin; FOS, GOS, 6′SL, and inulin; FOS, 2′FL, 6′SL, and inulin; FOS, LNnT, 6′SL, and inulin; GOS, 2′FL, 6′SL, and inulin; GOS, LNnT, 6′SL, and inulin; 2′FL, LNnT, 6′SL, and inulin; FOS, 6′SL, and inulin; GOS, 6′SL, and inulin; FOS, GOS, 2′FL, LNnT, 3′SL, 6′SL, and polydextrose; FOS, GOS, 2′FL, 3′SL, 6′SL, and polydextrose; FOS, GOS, LNnT, 3′SL, 6′SL, and polydextrose; FOS, 2′FL, LNnT, 3′SL, 6′SL, and polydextrose; GOS, 2′FL, LNnT, 3′SL, 6′SL, and polydextrose; FOS, GOS, 3′SL, 6′SL, and polydextrose; FOS, 2′FL, 3′SL, 6′SL, and polydextrose; FOS, LNnT, 3′SL, 6′SL, and polydextrose; GOS, 2′FL, 3′SL, 6′SL, and polydextrose; GOS, LNnT, 3′SL, 6′SL, and polydextrose; 2′FL, LNnT, 3′SL, 6′SL, and polydextrose; FOS, 3′SL, 6′SL, and polydextrose; GOS, 3′SL, 6′SL, and polydextrose; 2′FL, 3′SL, 6′SL, and polydextrose; LNnT, 3′SL, 6′SL, and polydextrose; FOS, GOS, 2′FL, LNnT, 3′SL, and polydextrose; FOS, GOS, 2′FL, 3′SL, and polydextrose; FOS, GOS, LNnT, 3′SL, and polydextrose; FOS, 2′FL, LNnT, 3′SL, and polydextrose; GOS, 2′FL, LNnT, 3′SL, and polydextrose; FOS, GOS, 3′SL, and polydextrose; FOS, 2′FL, 3′SL, and polydextrose; FOS, LNnT, 3′SL, and polydextrose; GOS, 2′FL, 3′SL, and polydextrose; GOS, LNnT, 3′SL, and polydextrose; 2′FL, LNnT, 3′SL, and polydextrose; FOS, 3′SL, and polydextrose; GOS, 3′SL, and polydextrose; 2′FL, 3′SL, and polydextrose; LNnT, 3′SL, and polydextrose; FOS, GOS, 2′FL, LNnT, 6′SL, and polydextrose; FOS, GOS, 2′FL, 6′SL, and polydextrose; FOS, GOS, LNnT, 6′SL, and polydextrose; FOS, 2′FL, LNnT, 6′SL, and polydextrose; GOS, 2′FL, LNnT, 6′SL, and polydextrose; FOS, GOS, 6′SL, and polydextrose; FOS, 2′FL, 6′SL, and polydextrose; FOS, LNnT, 6′SL, and polydextrose; GOS, 2′FL, 6′SL, and polydextrose; GOS, LNnT, 6′SL, and polydextrose; 2′FL, LNnT, 6′SL, and polydextrose; FOS, 6′SL, and polydextrose; GOS, 6′SL, and polydextrose; 2′FL, 6′SL, and polydextrose; LNnT, 6′SL, and polydextrose; FOS, GOS, 2′FL, LNnT, 3′SL, 6′SL, and a gum; FOS, GOS, 2′FL, 3′SL, 6′SL, and a gum; FOS, GOS, LNnT, 3′SL, 6′SL, and a gum; FOS, 2′FL, LNnT, 3′SL, 6′SL, and a gum; GOS, 2′FL, LNnT, 3′SL, 6′SL, and a gum; FOS, GOS, 3′SL, 6′SL, and a gum; FOS, 2′FL, 3′SL, 6′SL, and a gum; FOS, LNnT, 3′SL, 6′SL, and a gum; GOS, 2′FL, 3′SL, 6′SL, and a gum; GOS, LNnT, 3′SL, 6′SL, and a gum; 2′FL, LNnT, 3′SL, 6′SL, and a gum; FOS, 3′SL, 6′SL, and a gum; GOS, 3′SL, 6′SL, and a gum; 2′FL, 3′SL, 6′SL, and a gum; LNnT, 3′SL, 6′SL, and a gum; FOS, GOS, 2′FL, LNnT, 3′SL, and a gum; FOS, GOS, 2′FL, 3′SL, and a gum; FOS, GOS, LNnT, 3′SL, and a gum; FOS, 2′FL, LNnT, 3′SL, and a gum; GOS, 2′FL, LNnT, 3′SL, and a gum; FOS, GOS, 3′SL, and a gum; FOS, 2′FL, 3′SL, and a gum; FOS, LNnT, 3′SL, and a gum; GOS, 2′FL, 3′SL, and a gum; GOS, LNnT, 3′SL, and a gum; 2′FL, LNnT, 3′SL, and a gum; FOS, 3′SL, and a gum; GOS, 3′SL, and a gum; 2′FL, 3′SL, and a gum; LNnT, 3′SL, and a gum; FOS, GOS, 2′FL, LNnT, 6′SL, and a gum; FOS, GOS, 2′FL, 6′SL, and a gum; FOS, GOS, LNnT, 6′SL, and a gum; FOS, 2′FL, LNnT, 6′SL, and a gum; GOS, 2′FL, LNnT, 6′SL, and a gum; FOS, GOS, 6′SL, and a gum; FOS, 2′FL, 6′SL, and a gum; FOS, LNnT, 6′SL, and a gum; GOS, 2′FL, 6′SL, and a gum; GOS, LNnT, 6′SL, and a gum; 2′FL, LNnT, 6′SL, and a gum; FOS, 6′SL, and a gum; GOS, 6′SL, and a gum; 2′FL, 6′SL, and a gum; and LNnT, 6′SL, and a gum.

(14) Probiotics

The nutritional powders of the present disclosure may, in addition to HMOs (and, optionally, other prebiotic oligosaccharides as described above), comprise one or more probiotics. In some embodiments, the nutritional powder includes a combination of HMOs and probiotics such that the powder (and ultimate nutritional formula) provides a synergistic benefit to the end user in promoting the growth of microbiota in the gastrointestinal tract of infants.

Probiotics are live microorganisms thought to be healthy for the host organism. Lactic acid bacteria (LAB) and bifidobacteria are the most common types of microbes used as probiotics. Probiotics maintain the microbial ecology of the gut and show physiological, immuno-modulatory and antimicrobial effects, such that the use of probiotics has been found to prevent and treat gastrointestinal diseases and/or disorders, pathogen-induced diarrhea and toxin-producing bacteria, urogenital infections, and atopic diseases.

In order for microbes to exhibit beneficial probiotic effects in vivo, the organisms should survive for extended time periods in the gastrointestinal tract. Therefore, it is important that probiotic strains be selected that possess qualities that prevent their rapid removal by gut contraction. Effective probiotic strains are able to survive gastric conditions and colonize the intestine, at least temporarily, by adhering to the intestinal epithelium.

Non-limiting examples of probiotic strains for use in the nutritional powders herein include the genus Lactobacillus including L. acidophilus (e.g., L. acidophilus LA-5 and L. acidophilus NCFM), L. amylovorus, L. brevis, L. bulgaricus, L. casei spp. casei, L. casei spp. rhamnosus, L. crispatus, L. delbrueckii ssp. lactis, L. fermentum (e.g., L. fermentum CETC5716), L. helveticus, L. johnsonii, L. paracasei, L. pentosus, L. plantarum, L. reuteri (e.g., L. reuteri ATCC 55730, L. reuteri ATCC PTA-6475, and L. reuteri DSM 17938), L. sake, and L. rhamnosus (e.g., L. rhamnosus LGG and L. rhamnosus HN001); the genus Bifidobacterium including: B. animalis (e.g., B. animalis spp. lactis Bb-12), B. bifidum, B. breve (e.g., B. breve M-16V), B. infantis (e.g., B. infantis M-63, B. infantis ATCC 15697, B. Infantis 35624, B. infantis CHCC2228, B. infantis BB-02, B. infantis DSM20088, and B. infantis R-0033), B. longum (e.g., B. longum BB536, B. longum AH1205, and B. longum AH1206), and B. lactis (e.g., B. lactis HNO19 and B. lactis Bi07); the genus Pediococcus including: P. acidilactici; the genus Propionibacterium including: P. acidipropionici, P. freudenreichii, P. jensenii, and P. theonii; and the genus Streptococcus including: S. cremoris, S. lactis, and S. thermophilus. Particularly preferred probiotics include probiotics of human infant origin such as B. infantis M-63 and B. infantis ATCC 15697.

The probiotic is present in the nutritional powders in a total amount of at least about 10³ CFU/g, including from about 10³ CFU/g to about 10¹² CFU/g, and including from about 10⁶ CFU/g to about 10⁷ CFU/g.

In some embodiments, the nutritional powder includes a probiotic in combination with a first oligosaccharide including fructooligosaccharide and/or a galactooligosaccharide further in combination with a second oligosaccharide including at least one HMO such as 2′FL, 3′FL, 3′SL, 6′SL, and/or LNnT. In these embodiments, the first oligosaccharide and the second oligosaccharide are present in the powders in a weight ratio of first oligosaccharide:second oligosaccharide of about 10:1, or even from about 11:1 to about 8:1.

(15) Optional Ingredients

The nutritional powders described herein may further comprise other optional ingredients that may modify the physical, chemical, hedonic or processing characteristics of the products or serve as additional nutritional components when used for a targeted population. Many such optional ingredients are known or otherwise suitable for use in other nutritional products and may also be used in the nutritional powders described herein, provided that such optional ingredients are safe and effective for oral administration and are compatible with the essential and other ingredients in the selected product form.

Non-limiting examples of such optional ingredients include preservatives, antioxidants, emulsifying agents, buffers, additional nutrients as described herein, colorants, flavors, thickening agents, stabilizers, and so forth.

The nutritional powders may further comprise minerals, non-limiting examples of which include calcium, phosphorus, magnesium, iron, zinc, manganese, copper, sodium, potassium, molybdenum, chromium, selenium, chloride, and combinations thereof

The nutritional powders may further comprise vitamins or related nutrients, non-limiting examples of which include vitamin A, vitamin D, vitamin E, vitamin K, thiamine, riboflavin, pyridoxine, vitamin B12, other carotenoids, niacin, folic acid, pantothenic acid, biotin, vitamin C, choline, inositol, salts and derivatives thereof, and combinations thereof

In some embodiments, the nutritional powders may comprise a compound selected from the group of beta-hydroxyl beta-methyl butyrate, L-leucine, beta-alanine, epigallocatechin gallate, human milk oligosaccharides, prebiotics, probiotics, and combinations thereof.

The nutritional powders may also include one or more masking agents to reduce or otherwise obscure bitter flavors and after taste. Suitable masking agents include natural and artificial sweeteners, sodium sources such as sodium chloride, and hydrocolloids, such as guar gum, xanthan gum, carrageenan, gellan gum, and combinations thereof. The amount of masking agent in the nutritional powder may vary depending upon the particular masking agent selected, other ingredients in the nutritional powder, and other nutritional powder or product target variables. Such amounts, however, most typically range from at least 0.1 wt %, including from about 0.15 wt % to about 3.0 wt %, and also including from about 0.18 wt % to about 2.5 wt %, by weight of the nutritional powder.

b. Pod

The nutrient delivery system may comprise a disposable dispenser container or pod having a container body and a flexible lid that collectively define an enclosed volume. The pod contains the nutritional powder. The nutrient delivery system provides water at a particular temperature as indicated above to the pod, to create a mixture of the nutritional powder and water and thereby provide the nutritional formula. The nutritional formula is delivered from the pod to a receptacle such as a cup or baby bottle by the nutrient delivery system. In some embodiments, a stick pack can be used in place of a pod.

A pod is a disposable container having a container body and a flexible lid that collectively define an enclosed volume. The container body includes a generally arcuate bottom wall and a side wall extending from and integrally formed as one piece with the bottom wall and terminating in a generally flat rim or flange at an open upper end of the container. The enclosed volume may range from approximately 60 milliliters (mL) to approximately 500 mL, e.g., from approximately 60 mL to approximately 170 mL, or from approximately 80 mL to approximately 100 mL, in one or more chambers. For example, the volume may be approximately 60 mL, 61 mL, 62 mL, 63 mL, 64 mL, 65 mL, 66 mL, 67 mL, 68 mL, 69 mL, 70 mL, 71 mL, 72 mL, 73 mL, 74 mL, 75 mL, 76 mL, 77 mL, 78 mL, 79 mL, 80 mL, 81 mL, 82 mL, 83 mL, 84 mL, 85 mL, 86 mL, 87 mL, 88 mL, 89 mL, 90 mL, 91 mL, 92 mL, 93 mL, 94 mL, 95 mL, 96 mL, 97 mL, 98 mL, 99 mL, 100 mL, 101 mL, 102 mL, 103 mL, 104 mL, 105 mL, 106 mL, 107 mL, 108 mL, 109 mL, 110 mL, 111 mL, 112 mL, 113 mL, 114 mL, 115 mL, 116 mL, 117 mL, 118 mL, 119 mL, 120 mL, 121 mL, 122 mL, 123 mL, 124 mL, 125 mL, 126 mL, 127 mL, 128 mL, 129 mL, 130 mL, 131 mL, 132 mL, 133 mL, 134 mL, 135 mL, 136 mL, 137 mL, 138 mL, 139 mL, 140 mL, 141 mL, 142 mL, 143 mL, 144 mL, 145 mL, 146 mL, 147 mL, 148 mL, 149 mL, 150 mL, 151 mL, 152 mL, 153 mL, 154 mL, 155 mL, 156 mL, 157 mL, 158 mL, 159 mL, 160 mL, 161 mL, 162 mL, 163 mL, 164 mL, 165 mL, 166 mL, 167 mL, 168 mL, 169 mL, 170 mL, 171 mL, 172 mL, 173 mL, 174 mL, 175 mL, 176 mL, 177 mL, 178 mL, 179 mL, 180 mL, 181 mL, 182 mL, 183 mL, 184 mL, 185 mL, 186 mL, 187 mL, 188 mL, 189 mL, 190 mL, 191 mL, 192 mL, 193 mL, 194 mL, 195 mL, 196 mL, 197 mL, 198 mL, 199 mL, 200 mL, 210 mL, 220 mL, 230 mL, 240 mL, 250 mL, 260 mL, 270 mL, 280 mL, 290 mL, 300 mL, 310 mL, 320 mL, 330 mL, 340 mL, 350 mL, 360 mL, 370 mL, 380 mL, 390 mL, 400 mL, 410 mL, 420 mL, 430 mL, 440 mL, 450 mL, 460 mL, 470 mL, 480 mL, 490 mL, or 500 mL. The bottom wall and side wall together define an internal surface having an area ranging from approximately 20 square centimeters (cm²) to approximately 75 cm², and preferably between approximately 24 cm² and approximately 60 cm². For example, the area may be 20 cm², 21 cm², 22 cm², 23 cm², 24 cm², 25 cm², 26 cm², 27 cm², 28 cm², 29 cm², 30 cm², 31 cm², 32 cm², 33 cm², 34 cm², 35 cm², 36 cm², 37 cm², 38 cm², 39 cm², 40 cm², 41 cm², 42 cm², 43 cm², 44 cm², 45 cm², 46 cm², 47 cm², 48 cm², 49 cm², 50 cm², 51 cm², 52 cm², 53 cm², 54 cm², 55 cm², 56 cm², 57 cm², 58 cm², 59 cm², 60 cm², 61 cm², 62 cm², 63 cm², 64 cm², 65 cm², 66 cm², 67 cm², 68 cm², 69 cm², 70 cm², 71 cm², 72 cm², 73 cm², 74 cm², 75 cm², 76 cm², 77 cm², 78 cm², 79 cm², 80 cm², 81 cm², 82 cm², 83 cm², 84 cm², 85 cm², 86 cm², 87 cm², 88 cm², 89 cm², 90 cm², 91 cm², 92 cm², 93 cm², 94 cm², 95 cm², 96 cm², 97 cm², 98 cm², 99 cm², 100 cm², 101 cm², 102 cm², 103 cm², 104 cm², 105 cm², 106 cm², 107 cm², 108 cm², 109 cm², 110 cm², 111 cm², 112 cm², 113 cm², 114 cm², 115 cm², 116 cm², 117 cm², 118 cm², 119 cm², 120 cm², 121 cm², 122 cm², 123 cm², 124 cm², 125 cm², 126 cm², 127 cm², 128 cm², 129 cm², 130 cm², 131 cm², 132 cm², 133 cm², 134 cm², 135 cm², 136 cm², 137 cm², 138 cm², 139 cm², 140 cm², 141 cm², 142 cm², 143 cm², 144 cm², 145 cm², 146 cm², 147 cm², 148 cm², 149 cm², 150 cm², 151 cm², 152 cm², 153 cm², 154 cm², 155 cm², 156 cm², 157 cm², 158 cm², 159 cm², or 160 cm².

The pod is sized to receive from approximately 2 grams to approximately 150 grams, or between approximately 7.5 grams and approximately 35 grams, of a substantially soluble nutritional powder or liquid concentrate through the open upper end, after which the lid is hermetically sealed to the flange. For example, the pod may receive approximately 2.0 g, 2.5 g, 3.0 g, 3.5 g, 4.0 g, 4.5 g, 5.0 g, 5.5 g, 6.0 g, 6.5 g, 7.0 g, 7.5 g, 8.0 g, 8.5 g, 9.0 g, 9.5 g, 10 g, 10.5 g, 11 g, 11.5 g, 12 g, 12.5 g, 13 g, 13.5 g, 14 g, 14.5 g, 15 g, 15.5 g, 16 g, 16.5 g, 17 g, 17.5 g, 18 g, 18.5 g, 19 g, 19.5 g, 20 g, 20.5 g, 21 g, 21.5 g, 22 g, 22.5 g, 23 g, 23.5 g, 24 g, 24.5 g, 25 g, 25.5 g, 26 g, 26.5 g, 27 g, 27.5 g, 28 g, 28.5 g, 29 g, 29.5 g, 30 g, 30.5 g, 31 g, 31.5 g, 32 g, 32.5 g, 33 g, 33.5 g, 34 g, 34.5 g, 35 g, 35.5 g, 36 g, 36.5 g, 37 g, 37.5 g, 38 g, 38.5 g, 39 g, 39.5 g, 40 g, 40.5 g, 41 g, 41.5 g, 42 g, 42.5 g, 43 g, 43.5 g, 44 g, 44.5 g, 45 g, 45.5 g, 46 g, 46.5 g, 47 g, 47.5 g, 48 g, 48.5 g, 49 g, 49.5 g, 50 g, 50.5 g, 51.0 g, 51.5 g, 52.0 g, 52.5 g, 53.0 g, 53.5 g, 54.0 g, 54.5 g, 55.0 g, 55.5 g, 56.0 g, 56.5 g, 57.0 g, 57.5 g, 58.0 g, 58.5 g, 59.0 g, 59.5 g, 60.0 g, 60.5 g, 61.0 g, 61.5 g, 62.0 g, 62.5 g, 63.0 g, 63.5 g, 64.0 g, 64.5 g, 65.0 g, 65.5 g, 66.0 g, 66.5 g, 67.0 g, 67.5 g, 68.0 g, 68.5 g, 69.0 g, 69.5 g, 70.0 g, 70.5 g, 71.0 g, 71.5 g, 72.0 g, 72.5 g, 73.0 g, 73.5 g, 74.0 g, 74.5 g, 75.0 g, 75.5 g, 76.0 g, 76.5 g, 77.0 g, 77.5 g, 78.0 g, 78.5 g, 79.0 g, 79.5 g, 80.0 g, 80.5 g, 81.0 g, 81.5 g, 82.0 g, 82.5 g, 83.0 g, 83.5 g, 84.0 g, 84.5 g, 85.0 g, 85.5 g, 86.0 g, 86.5 g, 87.0 g, 87.5 g, 88.0 g, 88.5 g, 89.0 g, 89.5 g, 90.0 g, 90.5 g, 91.0 g, 91.5 g, 92.0 g, 92.5 g, 93.0 g, 93.5 g, 94.0 g, 94.5 g, 95.0 g, 95.5 g, 96.0 g, 96.5 g, 97.0 g, 97.5 g, 98.0 g, 98.5 g, 99.0 g, 99.5 g, 100.0 g, 100.5 g, 101.0 g, 101.5 g, 102.0 g, 102.5 g, 103.0 g, 103.5 g, 104.0 g, 104.5 g, 105.0 g, 105.5 g, 106.0 g, 106.5 g, 107.0 g, 107.5 g, 108.0 g, 108.5 g, 109.0 g, 109.5 g, 110 g, 110.5 g, 111 g, 111.5 g, 112 g, 112.5 g, 113 g, 113.5 g, 114 g, 114.5 g, 115 g, 115.5 g, 116 g, 116.5 g, 117 g, 117.5 g, 118 g, 118.5 g, 119 g, 119.5 g, 120 g, 120.5 g, 121 g, 121.5 g, 122 g, 122.5 g, 123 g, 123.5 g, 124 g, 124.5 g, 125 g, 125.5 g, 126 g, 126.5 g, 127 g, 127.5 g, 128 g, 128.5 g, 129 g, 129.5 g, 130 g, 130.5 g, 131 g, 131.5 g, 132 g, 132.5 g, 133 g, 133.5 g, 134 g, 134.5 g, 135 g, 135.5 g, 136 g, 136.5 g, 137 g, 137.5 g, 138 g, 138.5 g, 139 g, 139.5 g, 140 g, 140.5 g, 141 g, 141.5 g, 142 g, 142.5 g, 143 g, 143.5 g, 144 g, 144.5 g, 145 g, 145.5 g, 146 g, 146.5 g, 147 g, 147.5 g, 148 g, 148.5 g, 149 g, 149.5 g, or 150 g of the substantially soluble nutritional powder or liquid concentrate. The substantially soluble nutritional powder or liquid concentrate may occupy about 60% to about 90% of the volume of the pod, e.g., about 60%, 65%, 70%, 75%, 80%, 85% or 90% of the volume of the pod.

The container body is molded or otherwise constructed of a food-safe plastic material, such as polypropylene or polyethylene. The lid can be made of a polymer film, metal foil, or any other material suitable for affixing to the flange. At least one of the lid and the container body is configured to receive an injector or similar device through which water, air, or other fluids may be introduced to facilitate mixing and reconstitution within the enclosed volume. The introduced fluid(s) may be pre-filtered or alternatively pass through a filtration unit disposed within the container. An outlet member integrally formed as part of or movably coupled to the container body is positioned for dispensing from the pod, with the assistance of the introduced fluid(s), a nutritional product incorporating the powder or liquid concentrate. The dispensed product volume can range from approximately 5 mL to approximately 1000 mL, for example from approximately 20 mL to approximately 750 mL, from approximately 50 mL to approximately 500 mL, and is preferably between approximately 70 mL and approximately 250 mL. For example, the dispensed product volume may be approximately 5 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 105 mL, 110 mL, 115 mL, 120 mL, 125 mL, 130 mL, 135 mL, 140 mL, 145 mL, 150 mL, 155 mL, 160 mL, 165 mL, 170 mL, 175 mL, 180 mL, 185 mL, 190 mL, 195 mL, 200 mL, 205 mL, 210 mL, 215 mL, 220 mL, 225 mL, 230 mL, 235 mL, 240 mL, 245 mL, 250 mL, 255 mL, 260 mL, 265 mL, 270 mL, 275 mL, 280 mL, 290 mL, 300 mL, 305 mL, 310 mL, 315 mL, 320 mL, 325 mL, 330 mL, 335 mL, 340 mL, 345 mL, 350 mL, 355 mL, 360 mL, 365 mL, 370 mL, 375 mL, 380 mL, 385 mL, 390 mL, 395 mL, 400 mL, 405 mL, 410 mL, 415 mL, 420 mL, 425 mL, 430 mL, 435 mL, 440 mL, 445 mL, 450 mL, 455 mL, 460 mL, 465 mL, 470 mL, 475 mL, 480 mL, 490 mL, 500 mL, 505 mL, 510 mL, 515 mL, 520 mL, 525 mL, 530 mL, 535 mL, 540 mL, 545 mL, 550 mL, 555 mL, 560 mL, 565 mL, 570 mL, 575 mL, 580 mL, 585 mL, 590 mL, 595 mL, 600 mL, 605 mL, 610 mL, 615 mL, 620 mL, 625 mL, 630 mL, 635 mL, 640 mL, 645 mL, 650 mL, 655 mL, 660 mL, 665 mL, 670 mL, 675 mL, 680 mL, 690 mL, 700 mL, 705 mL, 710 mL, 715 mL, 720 mL, 725 mL, 730 mL, 735 mL, 740 mL, 745 mL, 750 mL, 755 mL, 760 mL, 765 mL, 770 mL, 775 mL, 780 mL, 785 mL, 790 mL, 795 mL, 800 mL, 805 mL, 810 mL, 815 mL, 820 mL, 825 mL, 830 mL, 835 mL, 840 mL, 845 mL, 850 mL, 855 mL, 860 mL, 865 mL, 870 mL, 875 mL, 880 mL, 890 mL, 900 mL, 905 mL, 910 mL, 915 mL, 920 mL, 925 mL, 930 mL, 935 mL, 940 mL, 945 mL, 950 mL, 955 mL, 960 mL, 965 mL, 970 mL, 975 mL, 980 mL, 985 mL, 990 mL, 995 mL, or 1000 mL. The temperature of the dispensed nutritional product is product dependent and can range from approximately 5° C. to approximately 60° C., or from approximately 25° C. to about 50° C. For example, the temperature may be approximately 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C.

c. Nutritional Formula

As discussed above, the nutrient delivery system may comprise a nutritional powder that is within a pod. The nutrient delivery system delivers water at a particular temperature to the nutritional powder within the pod, and provides a nutritional formula. The nutritional formula is delivered from the pod to a receptacle such as a cup or baby bottle. The physical characteristics that are important for the overall function of the nutritional formula include the powder reconstitution characteristics (e.g., wettability), viscosity, foaming, emulsion stability, amino acid profile, mineral delivery, antioxidant capacity, shelf-life stability, odor, flavor, and digestibility.

(1) Viscosity

The nutritional formula may comprise a viscosity of about 0.8 to about 30 cPs, or about 0.8 to about 10 cPs. Viscosity is the measurement of resistance to gradual deformation by shear or tensile stress. The nutritional formula's viscosity may be dependent on the components that are comprised within the nutritional composition. The viscosity of the nutritional formula is important on the overall flow performance of the nutritional formula through the nutrient delivery system. The viscosity of the nutritional formula may be measured by a rheometer, which may be used to measure how a liquid, slurry, or suspension flows in response to applied forces. The rheometer may be a shear/rotational rheometer or an extensional rheometer. The shear/rotational rheometer may be a pipe/capillary rheometer, cone and plate rheometer, or linear shear rheometer. The extensional rheometer may be an acoustic rheometer, falling plate rheometer, or capillary/contraction flow rheometer. The viscosity of the nutritional formula may be about 0.8 to 30 cPs, about 0.8 to 10 cPs, about 1 to 9 cPs, or about 2 to 6 cPs. The viscosity of the nutritional formula may be less than 1cPs, less than 2 cPs, less than 3 cPs, less than 4 cPs, less than 5 cPs, less than 6 cPs, less than 7 cPs, less than 8 cPs, less than 9 cPs, less than 10 cPs, less than 11 cPs, less than 12 cPs, less than 13 cPs, less than 14 cPs, less than 15 cPs, less than 16 cPs, less than 17 cPs, less than 18 cPs, less than 19 cPs, less than 20 cPs, less than 21 cPs, less than 22 cPs, less than 23 cPs, less than 24 cPs, less than 25 cPs, less than 26 cPs, less than 27 cPs, less than 28 cPs, less than 29 cPs, less than 30 cps, about 0.8 cPs, about 1 cPs, about 2 cPs, about 3 cPs, about 4 cPs, about 5 cPs, about 6 cPs, about 7 cPs, about 8 cPs, about 9 cPs, about 10 cPs, about 11 cPs, about 12 cPs, about 13 cPs, about 14 cPs, about 15 cPs, about 16 cPs, about 17 cPs, about 18 cPs, about 19 cPs, about 20 cPs, about 21 cPs, about 22 cPs, about 23 cPs, about 24 cPs, about 25 cPs, about 26 cPs, about 27 cPs, about 28 cPs, about 29 cPs, or about 30 cPs.

(2) Density

The nutritional formula may comprise a density between about 0.90 g/cm³ and about 1.2 g/cm³. The density of the nutritional formula is a function of the amount of entrapped air is present within the formula, among other factor, such as the compounds within the nutritional formula. The density of the nutritional formula is important in determining the flow characteristics of the formula, as well as well as side-effects associated with consumption of the formula (e.g., gassiness). The density of the nutritional formula may be about0.90 g/cm³, 0.91 g/cm³, 0.92 g/cm³, 0.93 g/cm³, 0.94 g/cm³, 0.95 g/cm³, 0.96 g/cm³, 0.97 g/cm³, 0.98 g/cm³, 0.99 g/cm³, 1.00 g/cm³, 1.01 g/cm³, 1.02 g/cm³, 1.03 g/cm³, 1.04 g/cm³, 1.05 g/cm³, 1.06 g/cm³, 1.07 g/cm³, 1.08 g/cm³, 1.09 g/cm³, 1.10 g/cm³, 1.11 g/cm³, 1.12 g/cm³, 1.13 g/cm³, 1.14 g/cm³, 1.15 g/cm³, 1.16 g/cm³, 1.17 g/cm³, 1.18 g/cm³, 1.19 g/cm³, or 1.20 g/cm³.

(3) Color Scale Values

The nutritional formula may comprise a Hunter Lab “L” value between about 20 and about 100. The Hunter Lab “L” value is a measurement of the lightness of the formula. The lightness of the nutritional formula is dependent on, but not limited to, the wettability, emulsion stability, and emulsion homogeneity. The Hunter Lab “L” value of the nutritional formula can be measured by a spectrophotometer, which allows quantitative measurement of the reflection or transmission properties of the formula as a function of wavelength. The Hunter Lab “L” value of the nutritional formula may be about 20.00, 25.00, 30.00, 35.00, 40.00, 45.00, 50.00, 55.00, 60.00, 65.00, 70.00, 75.00, 80.00, 80.10, 80.15, 80.20, 80.25, 80.30, 80.35, 80.40, 80.45, 80.50, 80.55, 80.60, 80.65, 80.70, 80.75, 80.80, 80.85, 80.90, 80.95, 81.00, 81.10, 81.15, 81.20, 81.25, 81.30, 81.35, 81.40, 81.45, 81.50, 81.55, 81.60, 81.65, 81.70, 81.75, 81.80, 81.85, 81.90, 81.95, 82.00, 82.10, 82.15, 82.20, 82.25, 82.30, 82.35, 82.40, 82.45, 82.50, 82.55, 82.60, 82.65, 82.70, 82.75, 82.80, 82.85, 82.90, 82.95, 83.00, 83.10, 83.15, 83.20, 83.25, 83.30, 83.35, 83.40, 83.45, 83.50, 83.55, 83.60, 83.65, 83.70, 83.75, 83.80, 83.85, 83.90, 83.95, 84.00, 86.00, 88.00, 90.00, 95.00 or 100.00.

The nutritional formula may comprise a Hunter Lab “a” value between about −5.00 and about 1.00. The Hunter Lab “a” value is a measurement of the color-opponent dimension of a formula. The “a” value of the nutritional formula is dependent on, but not limited to, the wettability, emulsion stability, and emulsion homogeneity. The Hunter Lab “a” value of the nutritional formula can be measured by a spectrophotometer, which allows quantitative measurement of the reflection or transmission properties of the formula as a function of wavelength. The Hunter Lab “a” value of the nutritional formula may be about −5.00, −4.50, −4.00, −3.50, −3.00, −2.50, −2.00, −1.50, −1.00, −0.50, −0.10, −0.09, −0.08, −0.07, −0.06, −0.05, −0.04, −0.03, −0.02, −0.01, 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.22, 0.24, 0.26, 0.28, 0.3, 0.35, 0.40, 0.45, 0.50, 0.55, 0.60, 0.65, 0.70, 0.75, 0.80, 0.85, 0.90, or 1.00.

The nutritional formula may comprise a Hunter Lab “b” value between about 1 and about 30. The Hunter Lab “b” value is a measurement of the color-opponent dimension of a formula. The “b” value of the nutritional formula is dependent on, but not limited to, the wettability, emulsion stability, and emulsion homogeneity. The Hunter Lab “b” value of the nutritional formula can be measured by a spectrophotometer, which allows quantitative measurement of the reflection or transmission properties of the formula as a function of wavelength. The Hunter Lab “b” value of the nutritional formula may be about 1.00, 2.00, 3.00, 4.00, 5.00, 6.00, 7.00, 8.00, 9.00, 10.00, 11.00, 12.00, 13.00, 13.10, 13.20, 13.30, 13.31, 13.32, 13.33, 13.34, 13.35, 13.36, 13.37, 13.38, 13.39, 13.40, 13.41, 13.42, 13.43, 13.44, 13.45, 13.46, 13.47, 13.48, 13.49, 13.50, 13.51, 13.52, 13.53, 13.54, 13.55, 13.56, 13.57, 13.58, 13.59, 13.60, 13.61, 13.62, 13.63, 13.64, 13.65, 13.66, 13.67, 13.68, 13.69, 13.70, 13.71, 13.72, 13.73, 13.74, 13.75, 13.76, 13.77, 13.78, 13.79, 13.80, 13.81, 13.82, 13.83, 13.84, 13.85, 13.86, 13.87, 13.88, 13.89, 13.90, 13.91, 13.92, 13.93, 13.94, 13.95, 13.96, 13.97, 13.98, 13.99, 14.00, 15.00, 16.00, 17.00, 18.00, 19.00, 20.00, 25.00, or 30.00.

(4) Caloric Density

The nutritional formula produced by the nutrient delivery system may comprise a caloric density of about 65 kcal/240 mL to about 800 kcal/240 mL. The nutritional formula, as discussed herein, provides a method to easily and effectively control caloric intake to an individual (e.g., infant). The ability to tightly control caloric intake is important because different individuals have different caloric needs. The nutritional formula produced by the nutrient delivery system may comprise a caloric density of about 65 kcal/240 mL, 70 kcal/240 mL, 75 kcal/240 mL, 80 kcal/240 mL, 85 kcal/240 mL, 90 kcal/240 mL, 95 kcal/240 mL, 100 kcal/240 mL, 105 kcal/240 mL, 110 kcal/240 mL, 115 kcal/240 mL, 120 kcal/240 mL, 125 kcal/240 mL, 130 kcal/240 mL, 135 kcal/240 mL,140 kcal/240 mL, 145 kcal/240 mL, 150 kcal/240 mL, 155 kcal/240 mL, 160 kcal/240 mL, 165 kcal/240 mL, 170 kcal/240 mL, 175 kcal/240 mL, 180 kcal/240 mL, 185 kcal/240 mL, 190 kcal/240 mL, 195 kcal/240 mL, 200 kcal/240 mL, 205 kcal/240 mL, 210 kcal/240 mL, 215 kcal/240 mL, 220 kcal/240 mL, 225 kcal/240 mL, 230 kcal/240 mL, 235 kcal/240 mL, 240 kcal/240 mL, 245 kcal/240 mL, 250 kcal/240 mL, 255 kcal/240 mL, 260 kcal/240 mL, 265 kcal/240 mL, 270 kcal/240 mL, 275 kcal/240 mL, 280 kcal/240 mL, 285 kcal/240 mL, 290 kcal/240 mL, 295 kcal/240 mL, 300 kcal/240 mL, 305 kcal/240 mL, 310 kcal/240 mL, 315 kcal/240 mL, 320 kcal/240 mL, 325 kcal/240 mL, 330 kcal/240 mL, 335 kcal/240 mL, 340 kcal/240 mL, 345 kcal/240 mL, 350 kcal/240 mL, 355 kcal/240 mL, 360 kcal/240 mL, 365 kcal/240 mL, 370 kcal/240 mL, 375 kcal/240 mL, 380 kcal/240 mL, 385 kcal/240 mL, 390 kcal/240 mL, 395 kcal/240 mL, 400 kcal/240 mL, 405 kcal/240 mL, 410 kcal/240 mL, 415 kcal/240 mL, 420 kcal/240 mL, 425 kcal/240 mL, 430 kcal/240 mL, 435 kcal/240 mL, 440 kcal/240 mL, 445 kcal/240 mL, 450 kcal/240 mL, 455 kcal/240 mL, 460 kcal/240 mL, 465 kcal/240 mL, 470 kcal/240 mL, 475 kcal/240 mL, 480 kcal/240 mL, 485 kcal/240 mL, 490 kcal/240 mL, 495 kcal/240 mL, 500 kcal/240 mL, 550 kcal/240 mL, 600 kcal/240 mL, 650 kcal/240 mL, 700 kcal/240 mL, 750 kcal/240 mL, or 800 kcal/240 mL.

(5) Dispersibility

The nutritional formula may comprise a qualitative dispersibility of about 1 to about 4. Dispersibility is way of determining the degree of firmness and solubility of particles within a product. Dispersibility is important in determining flow characteristics of the nutritional formula. Dispersibility is measured as how well the product will pass through the nipple of an infant bottle. For example, in a qualitative assay, an 8 ounce bottle may be prepared and shaken for 10 seconds. The product is then passed through an 80 mesh sieve and scored based on the number of remaining particles. The nutritional formula may comprise a qualitative dispersibility of about 1.0, 1.5, 2.0, 2.5, 3.0, 3.5, or 4.0. In a quantitative assay, the product is passed through an 80 mesh sieve as described above, and the total number of particles present on the sieve is measured using a mm stick and /or ruler. The size of the particles are then stratified into groups consisting of less than 1 mm, 1 mm, 2 mm, 3 mm, 4 mm, 5 mm and greater than 5 mm.

(6) Foaming

The nutritional formula may comprise a foaming volume of about 1 mL to about 70 mL, about 1 mL to about 30 mL, or about 1 mL to about 20 mL. Foaming of the nutritional formula can be attributed to the presence of entrapped air after the nutrient delivery system provides the nutritional formula, which is dependent on at least the compounds within the nutritional powder. Decreased foaming is a desired property of the nutritional formula because excess foam may increase potential side-effects that can be associated with consumption of the nutritional formula (e.g., gassiness, bloating, etc.). The foaming volume of the nutritional formula may be measured by a graduated cylinder after being provided by the nutrient delivery system. Additionally the foaming value may be measured at variable time points after initial addition to the graduated cylinder (e.g., 1 minute, 2 minutes, 3 minutes, 4 minutes, 5 minutes, 10 minutes, 15 minutes, or 30 minutes after the nutritional formula has been added to the graduated). The foaming volume may be about 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL, 42 mL, 43 mL, 44 mL, 45 mL, 46 mL, 47 mL, 48 mL, 49 mL, 50 mL, 51 mL, 52 mL, 53 mL, 54 mL, 55 mL, 56 mL, 57 mL, 58 mL, 59 mL, 60 mL, 61 mL, 62 mL, 63 mL, 64 mL, 65 mL, 66 mL, 67 mL, 68 mL, 69 mL, or 70 mL.

The nutritional formula may comprise a foaming ratio of about 1 to about 15. The foaming ratio of the nutritional formula is investigated by measuring the volume of foam within a graduated cylinder after being provided by the nutrient delivery system and at variable time points after initial addition to the graduated cylinder (e.g., 1 minute, 2 minutes, 3 minutes, 4 minutes, 5 minutes, 10 minutes, 15 minutes, or 30 minutes after the nutritional formula has been added to the graduated cylinder). The ratio of foaming level at time 0 and the variable time points are then measured for the sample. The foaming ratio of the nutritional formula may be about 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, or 15.

(7) Human Milk Oligosaccharides

The nutritional formulas of the present disclosure include at least one HMO, and in many embodiments, a combination of two or more HMOs, as described above.

In one specific embodiment of the present disclosure, a nutritional formula includes 2′FL, alone or in combination with other additional HMOs (e.g., the 2′FL may be combined with 3′SL and/or 6′SL in some specific embodiments). In one embodiment, the 2′FL is included in the nutritional formula in an amount of from about 0.001 mg/mL to about 20 mg/mL, including from about 0.001 mg/mL to about 10 mg/mL, including from about 0.01 mg/mL to about 20 mg/mL, including from about 0.001 mg/mL to about 1 mg/mL, including from about 0.001 mg/mL to less than 2 mg/mL, including from about 0.01 mg/mL to less than 2 mg/mL, and also including from about 0.02 mg/mL to less than 2 mg/mL. In another embodiment, the 2′FL is included in the nutritional formula in an amount of from about 0.001 mg/mL to about 20 mg/mL, including from about 0.01 mg/mL to about 20 mg/mL, including from greater than 2.5 mg/mL to 20 mg/mL, including from greater than 2.5 mg/mL to 19.8 mg/mL, including from greater than 2.5 mg/mL to 15 mg/mL, and including from greater than 2.5 mg/mL to 10 mg/mL.

In one specific embodiment, the nutritional formula includes 6′SL, alone or in combination with other HMOs, in an amount of from about 0.001 mg/mL to about 20 mg/mL, including from about 0.01 mg/mL to about 20 mg/mL, including from about 0.001 mg/mL to less than 0.25 mg/mL, and including from about 0.01 mg/mL to less than 0.25 mg/mL. In another embodiment, the nutritional formula includes 6′SL, alone or in combination with other HMOs, in an amount of from about 0.001 mg/mL to about 20 mg/mL, including from about 0.01 mg/mL to about 20 mg/mL, including from greater than 0.4 mg/mL to about 20 mg/mL, including from greater than 0.4 mg/mL to about 15 mg/mL, including from greater than 0.4 mg/mL to about 10 mg/mL, and including from greater than 0.5 mg/mL to about 1.0 mg/mL.

In another embodiment, the nutritional formula includes 3′SL, alone or in combination with other HMOs, in an amount of from about 0.001 mg/mL to about 20 mg/mL, including from about 0.01 mg/mL to about 20 mg/mL, including from about 0.01 mg/mL to less than 0.15 mg/mL, including from greater than 0.25 mg/mL to about 20 mg/mL, including from greater than 0.25 mg/mL to about 15 mg/mL, and including from greater than 0.25 mg/mL to about 10 mg/mL.

In one specific embodiment, the nutritional formula includes LNnT, alone or in combination with other HMOs, in an amount of from about 0.001 mg/mL to about 20 mg/mL, including from about 0.01 mg/mL to about 20 mg/mL, including from about 0.001 mg/mL to less than 0.2 mg/mL, including from about 0.01 mg/mL to less than 0.2 mg/mL, including from greater than 0.32 mg/mL to about 20 mg/mL, including from greater than 0.32 mg/mL to about 15 mg/mL, and including from greater than 0.32 mg/mL to about 10 mg/mL.

In some embodiments, the HMOs are used in combination to provide the desired immune enhancing effect. For example, in one embodiment, the nutritional formula includes 6′SL in combination with 3′SL in a total amount of HMO of from about 0.001 mg/mL to about 20 mg/mL, including from about 0.01 mg/mL to about 20 mg/mL, including from about 0.001 mg/mL to about 0.23 mg/mL, including from about 0.01 mg/mL to about 0.23 mg/mL, including from about 0.001 mg/mL to less than 0.15 mg/mL, and including from about 0.01 mg/mL to less than 0.15 mg/mL of the nutritional formula. In another embodiment, the nutritional formula includes 6′SL in combination with 3′SL in a total amount of HMO of from about 0.001 mg/mL to about 20 mg/mL, including from about 0.01 mg/mL to about 20 mg/mL and including greater than 0.65 mg/mL to about 20 mg/mL.

In one specific embodiment, the nutritional formula includes 6′SL, alone or in combination with other HMOs, in an amount of from about 0.001 mg/mL to about 20 mg/mL, including from about 0.01 mg/mL to about 20 mg/mL, including from about 0.001 mg/mL to less than 0.25 mg/mL, including from about 0.01 mg/mL to less than 0.25 mg/mL, including from greater than 0.4 mg/mL to about 20 mg/mL, and including from about 0.1 mg/mL to about 0.5 mg/mL.

In another embodiment, the nutritional formula includes 3′SL, alone or in combination with other HMOs, in an amount of from about 0.001 mg/mL to about 20 mg/mL, including from about 0.01 mg/mL to about 20 mg/mL, including from about 0.001 mg/mL to less than 0.15 mg/mL, including from about 0.01 mg/mL to less than 0.15 mg/mL, and including from greater than 0.25 mg/mL to about 20 mg/mL.

In one specific embodiment, the nutritional formula includes LNnT, alone or in combination with other HMOs, in an amount of from about 0.001 mg/mL to about 20 mg/mL, including from about 0.01 mg/mL to about 20 mg/mL, including from about 0.001 mg/mL to less than 0.2 mg/mL, including from about 0.01 mg/mL to less than 0.2 mg/mL, and including from greater than 0.32 mg/mL to about 20 mg/mL.

In another specific embodiment, the nutritional formula includes 3′FL, alone or in combination with other HMOs, in an amount of from about 0.001 mg/mL to about 20 mg/mL, including from about 0.01 mg/mL to about 20 mg/mL, including from about 0.001 mg/mL to less than 1 mg/mL, including from about 0.01 mg/mL to less than 1 mg/mL, and including from greater than 1.7 mg/mL to about 20 mg/mL.

In one specific embodiment, the nutritional formula includes 3′FL in combination with SA in a total amount of HMO of from about 0.001 mg/mL to about 20 mg/mL, including from about 0.01 mg/mL to about 20 mg/mL. In one embodiment, the nutritional formula includes 3′FL in an amount of from about 0.001 mg/mL to less than 1 mg/mL, including from 0.01 mg/mL to less than 1 mg/mL and SA in an amount of about 1 mg/mL.

In another embodiment, the nutritional formula includes 2′FL, alone or in combination with other HMOs, in an amount of from about 0.001 mg/mL to about 20 mg/mL, including from about 0.01 mg/mL to about 20 mg/mL, including from about 0.001 mg/mL to less than 2 mg/mL, including from about 0.01 mg/mL to less than 2 mg/mL, including from about 0.001 mg/mL to about 1 mg/mL, and including from about 0.01 mg/mL to about 1 mg/mL. In another embodiment, the nutritional formula includes 2′FL, alone or in combination with other HMOs, in an amount of from about 0.001 mg/mL to about 20 mg/mL, including from about 0.01 mg/mL to about 20 mg/mL and including greater than 2.5 mg/mL to about 20 mg/mL.

In one specific embodiment, the nutritional formula includes 2′FL in combination with 3′FL in a total amount of HMO of from 0.001 mg/mL to about 20 mg/mL, including from about 0.01 mg/mL to about 20 mg/mL.

In yet another embodiment, the nutritional formula includes a combination of 6′ SL, 2′FL, and LNnT in a total amount of HMO of from about 0.001 mg/mL to about 20 mg/mL, including from about 0.01 mg/mL to about 20 mg/mL.

(8) Additional Prebiotic Oligosaccharides

The nutritional formulas of the present disclosure may, in addition to the HMOs described above, comprise an additional source or sources of prebiotic oligosaccharides, as described above. The oligosaccharide blend is present in the nutritional formulas in a total amount of at least about 1 mg/mL, including from about 1 mg/mL to about 20 mg/mL, including from about 1 mg/mL to about 15 mg/mL, including from about 1 mg/mL to about 10 mg/mL, including from about 1 mg/mL to about 5 mg/mL In one embodiment, the oligosaccharide blend is present in the nutritional formula in a total amount of from about 1 mg/mL to about 4 mg/mL.

Typically, when used as an oligosaccharide blend, the nutritional powders, in addition to the HMO or HMOs, include at least one rapidly-fermented oligosaccharide, at least one medium-fermented oligosaccharide, and, optionally, at least one slowly-fermented oligosaccharide to provide a nutritional formula that is tolerated well by infants (i.e., reduced gassiness and/or stool frequency), as discussed above. When used in an oligosaccharide blend, the rapidly-fermented oligosaccharides can be included in the nutritional formulas in amounts of from about 0.05 mg/mL to about 20 mg/mL, including from about 0.5 mg/mL to about 15 mg/mL, including from about 0.5 mg/mL to about 10 mg/mL, including from about 1 mg/mL to about 15 mg/mL, including from about 1 mg/mL to about 10 mg/mL, including from about 2 mg/mL to about 8 mg/mL, and also including from about 3 mg/mL to about 5 mg/mL. The medium-fermented oligosaccharides can be included in the nutritional formulas in amounts of from about 0.05 mg/mL to about 20 mg/mL, including from about 0.05 mg/mL to about 15 mg/mL, including from about 0.05 mg/mL to about 10 mg/mL, including from about 0.05 mg/mL to about 5 mg/mL, including from about 0.05 mg/mL to about 2.5 mg/mL, including from about 0.05 mg/mL to about 1 mg/mL, including from about 0.05 mg/mL to about 0.5 mg/mL, and including from about 0.05 mg/mL to about 0.25 mg/mL. The slowly-fermented oligosaccharides can be included in the nutritional formulas in amounts of from about 0.05 mg/mL to about 20 mg/mL, including from about 0.05 mg/mL to about 15 mg/mL, including from about 0.05 mg/mL to about 10 mg/mL, including from about 0.05 mg/mL to about 5 mg/mL, and also including from about 0.05 mg/mL to about 2.5 mg/mL.

In one specific embodiment, the nutritional formula includes an oligosaccharide blend including LNnT, 6′SL and inulin in a total amount of oligosaccharide blend of from about 0.05 mg/mL to about 20 mg/mL.

In another specific embodiment, the nutritional formula includes an oligosaccharide blend including 2′FL, 6′SL and inulin in a total amount of oligosaccharide blend of from about 0.05 mg/mL to about 20 mg/mL.

(9) Anti-Microbial or Bacteriostatic Activity

The nutritional formulas of the present disclosure may have improved anti-microbial and/or bacteriostatic properties, compared to a nutritional formula that does not include one or more HMOs. For example, inclusion of one or more HMOs may have an improved ability to kill or otherwise inhibit the growth or proliferation of microbes (e.g., bacteria) compared to a nutritional formula that does not include one or more HMOs. In some embodiments, a nutritional formula may kill or inhibit the growth of abnormal gut flora or bacteria other than normal gut flora.

### 3. USE OF THE NUTRIENT DELIVERY SYSTEM

The nutrient delivery system, as described above, provides the nutritional formula. The present invention is also directed to a method of producing the nutritional formula. The method may include providing the pod and the nutritional powder described above. The nutritional powder may be positioned within the pod such that the nutritional powder is fully enclosed by the bottom wall, side wall, and lid of the pod. Accordingly, the nutritional powder and the lid may define therebetween the headspace of the pod. As described above, the headspace may include less than about 10% O₂.

The method also includes introducing the fluid into the pod to produce the nutritional formula. Introducing may include the lid receiving the injector or similar device as described above, through which the fluid is delivered into the pod. The fluid may include water. Additionally, the fluid may be introduced into the pod at a suitable temperature, such as a temperature described herein.

The method may further include expelling the nutritional formula from the pod. Expelling may include passing the nutritional formula through the outlet port of the pod and into a container (e.g., bottle, glass, and so forth) from which the subject consumes the nutritional formula.

In some embodiments, the contents of the pod (i.e., the nutritional powder) are intended to be processed (i.e., reconstituted into a liquid product suitable for oral consumption by a subject) within seconds after a hermetic seal of the pod is broken to allow liquid to flow therein, the content to flow therefrom, or a combination thereof In such embodiments, the pod will typically be a single-use, disposable container. In other embodiments, the pod is sealable or re-sealable and is capable of re-use. In certain embodiments where the pod is sealable or re-sealable, the contents of the pod (i.e., the nutritional powder) may be stored for a short time (typically hours or days) by the consumer prior to reconstituting into a liquid product and the pod may or may not be hermetically sealed at any point.

In some embodiments, any delay between the time the hermetic seal of the pod is disrupted and the initiation time is less than 1 second. In other embodiments, any delay between the time the hermetic seal of the pod is disrupted and the initiation time is less than 2 seconds. In other embodiments, any delay between the time the hermetic seal of the pod is disrupted and the initiation time is less than 3 seconds. In other embodiments, any delay between the time the hermetic seal of the pod is disrupted and the initiation time is less than 4 seconds. In other embodiments, any delay between the time the hermetic seal of the pod is disrupted and the initiation time is less than 5 seconds. In other embodiments, any delay between the time the hermetic seal of the pod is disrupted and the initiation time is within the range of 1 second to 10 seconds. In some embodiments, a delay between the time the hermetic seal of the pod is disrupted and the initiation time is within the range of 1 second to 30 seconds.

In these embodiments, a subject desirably consumes at least one serving of the infant formula per day, and in some embodiments, may consume two, three, or even more servings per day. Each serving is desirably administered as a single undivided dose, although the serving may also be divided into two or more partial or divided servings to be taken at two or more times during the day. The methods of the present disclosure include continuous day after day administration, as well as periodic or limited administration, although continuous day after day administration is generally desirable.

The nutritional formulas as described herein can be used to address one or more of the diseases or conditions discussed herein, or can be used to provide one or more of the benefits described herein, to infants, toddlers, and children. The infant, toddler, or child utilizing the nutritional formulas described herein may actually have or be afflicted with the disease or condition described, or may be susceptible to, or at risk of, getting the disease or condition (that is, may not actually yet have the disease or condition, but is at elevated risk as compared to the general population for getting it due to certain conditions, family history, etc.) Whether the infant, toddler, or child actually has the disease or condition, or is at risk or susceptible to the disease or condition, the infant, toddler, or child is classified herein as “in need of assistance in dealing with and combating the disease or condition. For example, the infant, toddler, or child may actually have respiratory inflammation or may be at risk of getting respiratory inflammation (susceptible to getting respiratory inflammation) due to family history or other medical conditions, for example. Whether the infant, toddler, or child actually has the disease or condition, or is only at risk or susceptible to getting the disease or condition, it is within the scope of the present disclosure to assist the infant, toddler, or child with the nutritional formulas described herein.

Based on the foregoing, because some of the method embodiments of the present disclosure are directed to specific subsets or subclasses of identified individuals (that is, the subset or subclass of individuals “in need” of assistance in addressing one or more specific diseases or specific conditions noted herein), not all infants, toddlers, and children will fall within the subset or subclass of infants, toddlers, and children as described herein for certain diseases or conditions.

The nutritional formulas as described herein comprise HMOs, alone or in combination with one or more additional components, to provide a nutritional source for improving at least the intestinal/gut function. Specifically, the nutritional formulas may stimulate enteric nerve cells in the gastrointestinal tract of an individual to improve intestinal/gut barrier integrity; improve feeding tolerance (e.g., reduce feeding intolerance, reduce diarrhea, loose stools, gas, and bloating); reduce colic in infants; promote tolerance to enteral feeding, decrease time to full enteral feeding, increase the rate of advancement of enteral feeding, decrease the amount and duration of partial or total parenteral nutrition, protect against necrotizing enterocolitis and other disorders of prematurity; address gastrointestinal diseases and disorders associated with the enteric nervous system; address gastrointestinal diseases and disorders of gut contractility and inflammation; correct effects of gut dysbiosis; and affect long-term modulation of allergic tolerance.

More particularly, in some embodiments, the nutritional formulas may be administered to an individual having, susceptible to, or at risk of, gastrointestinal diseases and disorders associated with the enteric nervous system and/or associated with gut contractility and inflammation, which may include, for example, irritable bowel syndrome, colitis (e.g., necrotizing enterocolitis, Crohn's disease, ischemic colitis, cryptosporidium enterocolitis, pseudomembranous colitis, cytomegalovirus, ulcerative colitis), food intolerance, and food allergies.

Along with improved growth and maturation of an individual's immune system as described above, the use of the nutritional formulas of the present disclosure may also function to enhance the individual's ability to resist microbial infection and to promote the growth of beneficial microbiota in the gastrointestinal tract of an infant, toddler, child, or adult.

Additionally, the nutritional formulas of the present disclosure may also be used to improve cognition in individuals, particularly in individuals susceptible to, or at risk of, neurodegenerative diseases, which may include, for example, Alzheimer's disease, Huntington's disease, Parkinson's disease, and schizophrenia, or in individuals suffering from conditions caused by impaired cognitive development, or neurodevelopmental conditions, such as attention deficit hyperactivity disorder and autism.

The nutritional formulas as described herein may also provide a nutritional source for reducing inflammation, such as respiratory inflammation (e.g., respiratory syncytial virus-induced inflammation), enteric inflammation, and nasopharyngeal inflammation. The nutritional formulas of the present disclosure comprising HMOs may also provide optimal development and balanced growth and maturation of the infant's gastrointestinal and immune systems, thereby enhancing the infant's ability to resist microbial infection and modulate inflammatory responses to infection (e.g., increased phagocytosis and increased production of reactive oxidative species).

The nutritional formulas also provide growth and maturation of the intestinal epithelial cells in an infant. In one specific embodiment, the administration of the nutritional formulas of the present disclosure including HMOs and nucleotides can further activate immune activity in or by the intestinal epithelial cells in a newborn.

Further, the use of HMOs in nutritional formulas can reduce the growth of respiratory viruses (e.g., RSV, human parainfluenza virus type 2, and influenza A virus), and thus, reduce viral-induced upper respiratory infections. In addition, the use of HMOs in combination with nucleotides in nutritional formulas can reduce the growth of enteric viruses (e.g., rotavirus), and thus, reduce viral-induced enteric infections. As such, by utilizing HMOs, alone or in combination with other immune enhancing factors, in a nutritional product, such as an infant formula, it is now possible to provide infants with an alternative, or supplement, to breast milk that more closely mimics the benefits thereof

Along with improved growth and maturation of the infant's immune system as described above, the use of the nutritional formulas of the present disclosure also functions as an immune modulator, thereby reducing inflammation induced by infection in infants, toddlers, and children such as respiratory virus-induced infection, and particularly, RSV-induced inflammation, and other infection-mediated inflammatory diseases. By improving the growth and maturation of the immune system and reducing inflammation, the airway defense mechanisms of an infant, toddler, or child can be improved, thus improving the overall respiratory health of the infant, toddler, or child. Specifically, in some embodiments of the present disclosure, the HMO-containing nutritional formulas of the present disclosure can be used by an infant, toddler, or child to improve airway defense mechanisms. In other embodiments of the present disclosure, the HMO-containing nutritional formulas can be used by an infant, toddler, or child to improve overall airway respiratory health.

The addition of HMOs can further increase glutathione levels in the body and blood of an infant.

When used in combination with LCPUFAs and/or antioxidants, and particularly, with carotenoids, the HMOs can reduce oxidative stress, which is a metabolic condition in which there is an increased production and accumulation of oxidized biomolecules such as lipid peroxides and their catabolites, protein carbonyls, and oxidatively damaged DNA. The outcomes of oxidative stress range from unwanted changes in metabolism to inflammation and cell and tissue death. Accordingly, by reducing the incidence of unregulated inflammation and oxidation in the infant, damage to the tissue lining and cell death is reduced, further reducing the incidence of inflammatory diseases, such as necrotizing enterocolitis (NEC).

In addition to the benefits discussed above, it has been discovered that nutritional products including HMOs can modulate production of monocyte-derived cytokines in the infant, even in the absence of a virus. This production results in improved immunity to further prevent microbial infection and reduce the growth of viruses. In one specific embodiment, monocyte-derived cytokines produced by administration of the nutritional formulas of the present disclosure include, for example, interleukin-10, interleukin-8, interleukin-1α, interleukin-1β, interleukin-1ra, and combinations thereof.

Another benefit of utilizing HMOs in nutritional formulas is that it has been discovered that HMOs modulate the production of IP-10, which is a chemokine that plays an important role in the inflammatory response to viral infection. Specifically, a positive correlation exists between RSV clinical infection severity in children and serum IP-10 levels. Accordingly, a decrease in IP-10 signals a decrease in severity of RSV infection. In one specific embodiment, IP-10 production is reduced to the level found in uninfected controls.

Along with reducing IP-10, HMOs have been found to reduce platelet-neutrophil complex (PNC) formation, which is present in human blood and consists of up to 25% of unstimulated neutrophils. As PNCs are present in aggregates, they have a greater capacity to initiate inflammatory processes and can increase the production of reactive oxidative species. Accordingly, a decrease in PNC formation can lead to reduced oxidative stress and inflammation in the infant.

Further information regarding the uses of nutritional compositions comprising HMOs can be found, for example, in U.S. Patent Publication Nos. 2012/0171165, 2012/0171166, 2012/0172307, 2012/0172319, 2012/0172327, 2012/0172330, 2012/0172331, and 2012/0184503, the contents of each of which are hereby incorporated by reference.

### 4. METHOD OF MANUFACTURING THE NUTRITIONAL POWDER

The nutritional powders may be prepared by any known or otherwise effective technique suitable for making and formulating nutritional powders, variations of which may depend upon variables such as the selected ingredient combination, packaging and container selection, and so forth. Such techniques and variations are described in the nutritional art or are otherwise well known to those skilled in the nutritional art.

Methods of manufacturing nutritional powders typically involve the initial formation of an aqueous slurry containing carbohydrates, proteins, lipids, stabilizers or other formulation aids, vitamins, minerals, or combinations thereof. The slurry is emulsified, pasteurized, homogenized, and cooled. Various other solutions, mixtures, or other materials may be added to the resulting emulsion before, during, or after further processing. The resulting mixture is then heated and dried into powder form, which may be accomplished by spray drying or other heat-treating methods of forming solid particulates in a powder matrix. Other essential or optional materials may also be added to the powder by dry blending, agglomerating, or otherwise combining the added material to the forming or just formed solid particulates.

If dry blending is used as part of the formulation process, the type and amount of dry blended carbohydrates in a nutritional powder may be analyzed. Analysis may be performed using a microscope, by preparing a microscope slide with a sample of the powder and placing the slide under a standard stereoscopic microscope. The different types of particles are visually analyzed in terms of shape, size, color, and transparency, and measurements are recorded. Each different powder particle and test is extracted using infrared vibrational spectroscopy to confirm its identity.

Alternatively or as a complement to the above-described method, analysis may be done by static image analysis by testing a sample of the powder using an image analysis sensor (e.g., Malvern Morphologi G3). The analyzer provides a quantitative characterization of the different powder shapes and sizes.

Alternatively or as a complement to the above-listed methods, analysis may be done by via Differential Scanning calorimetry (DSC). A sample of powder is evaluated using a Differential Scanning calorimeter (e.g., TA Instruments' Q200). The analyzer provides a heat flow thermogram, which can differentiate 100% spray dried powders from partially or 100% dry blended powders from glass transition peaks.

The quantitative measurements from the static image analysis and DSC can be correlated to the different powder particles identified microscopically to calculate the type and amount of dry blended carbohydrates in the powder.

In one embodiment, a suitable manufacturing process may include the preparation of at least three separate slurries: a protein-in-fat (PIF) slurry, a carbohydrate-mineral (CHO-MIN) slurry, and a protein-in-water (PIW) slurry. The PIF slurry may be formed by heating and mixing the oil (e.g., canola oil, corn oil, soy oil, coconut oil, high oleic safflower oil) and then adding an emulsifier (e.g., lecithin), fat soluble vitamins, and a portion of the total protein (e.g., intact pea protein concentrate, milk protein concentrate, whey protein concentrate, nonfat milk) with continued heat and agitation. The CHO-MIN slurry may be formed by adding with heated agitation to water: minerals (e.g., potassium citrate, dipotassium phosphate, sodium citrate), trace and ultra trace minerals (TM/UTM premix), thickening or suspending agent. The resulting CHO-MIN slurry may be held for 10 minutes with continued heat and agitation before adding additional minerals (e.g., potassium chloride, magnesium carbonate, potassium iodide), and/or carbohydrates (e.g., HMOs, lactose, fructooligosaccharide, sucrose, corn syrup). The PIW slurry may then be formed by mixing with heat and agitation of the remaining protein, if any.

The resulting slurries are then blended together with heated agitation and the pH may be adjusted to the desired range, such as, from 6.6 to 7.5 (including 6.6 to 7), after which the nutritional emulsion is subjected to high-temperature short-time (“HTST”) processing (i.e., about 165° F. (74° C.) for about 16 seconds) or an ultra high temperature (UHT) processing step (i.e., about 292° F. (144° C.) for about 5 seconds). The nutritional emulsion is heat treated, emulsified, homogenized, and cooled during the HTST or UHT process. Water soluble vitamins and ascorbic acid are added (if applicable), the pH is again adjusted (if necessary). The batch is evaporated, heat treated and spray dried. After drying, the powder may be transported to storage hoppers. The base powder may be dry blended with the remaining ingredients to form the nutritional powder. The nutritional powder is then packaged in appropriate containers (i.e., pods, packages containing one or more pods, or kits containing one or more pods) for distribution. Those of skill in the art will understand that standard intermediate manufacturing steps, such as bulk storage, packing in large bags or drums, transport to other locations, etc., may be incorporated as part of this process.

In some embodiments, the nutritional emulsion is dried to form a nutritional powder using any methods known in the art. By way of example, nutritional powders can be manufactured by preparing at least two slurries, which are then mixed, heat treated, standardized, heat treated a second time, evaporated to remove water, and spray dried or dry blended to form a reconstitutable nutritional powder.

One exemplary method of preparing a spray dried nutritional powder suitable for use in the nutritional powder pods disclosed herein comprises forming and homogenizing an aqueous slurry or liquid comprising predigested fat, and optionally protein, carbohydrate, and other sources of fat, and then spray drying the slurry or liquid to produce a spray dried nutritional powder. The method may further comprise the step of spray drying, dry mixing, or otherwise adding additional nutritional ingredients, including any one or more of the ingredients described herein, to the spray dried nutritional powder.

Generally, when the nutritional powder for use in the nutritional powder pod is a spray dried nutritional powder or a dry blended nutritional powder, it may be prepared by any suitable known techniques. For example, the spray drying may include any spray drying technique that is suitable for use in the production of nutritional powders. Many different spray drying methods and techniques are known for use in the nutrition field, all of which are suitable for use in the manufacture of the spray dried nutritional powders herein. Following drying, the finished powder may be packaged into nutritional powder pods.

In other embodiments, the preparation of the nutritional powder comprises an extruded powder. Milling can also be included as a step in preparing the nutritional powder.

In some embodiments, the ingredients of the nutritional powder may be extruded as part of the process of making the nutritional powder. In certain embodiments, the ingredients are incorporated in the extruder hopper in the form of a dry feed or powder premix. The dry nutritional ingredients enter the extruder just after the point of entry of water. In certain embodiments, the water comprises from about 1% to about 80% by weight of the total weight of the water and dry ingredients. The amount of water added to the nutritional composition may be adjusted within the aforementioned ranges based on the desired physical properties of the extrudate. In certain embodiments, the nutritional ingredients may be premixed with water to form a thick emulsion, which is then fed into the extruder hopper in the form of a viscous liquid or sludge. The term “extrudate” refers to all or a portion of a nutritional composition that exits an extruder.

In some embodiments, the extruder used to produce the nutritional powder or extrudate operates in a continuous format. Generally, any extruder known for use in food processing may be utilized. In certain embodiments, extrusion is performed via a screw extruder. Said screw extruder may be a twin screw extruder or a single screw extruder. The extruder screws may consist of shear elements, mixing elements, conveying elements, kneading elements, emulsifying elements, disc elements, or a combination of the above in any interchangeable order. The barrels of the extruder may be steam heated or electrically heated. In certain embodiments, extrusion takes place at a temperature between about 20° C. to about 99° C., from about 30° C. to about 150° C., or from about 70° C. to about 100° C. In certain embodiments, the ingredients are processed in the extruder for about 5 seconds to about 240 seconds or for about 30 seconds to about 180 seconds.

In some embodiments disclosed herein, the extrudate is dried following extrusion so as to remove most or all of the water contained therein. In such embodiments, any conventional drying methods may be used to remove the desired amount of water from the nutritional powder. For example, the nutritional powder extrudate may be dried using a vacuum, convective hot air, a tray dryer, infrared, or any combination of the above. In some embodiments, the nutritional powder extrudate may be further ground or milled to a desired particle size following drying. In some embodiments, additional protein and carbohydrate ingredients may be added to the final nutritional powder in the form of dry ingredients or a dry blend.

In some embodiments, in order to increase or enhance the particle porosity of the nutritional powder, a pressurized gas may be introduced into the nutritional emulsion at a suitable time during the manufacturing process. This pressurized gas may dissolve into the nutritional emulsion during the blending stages if these stages are similarly conducted under pressure. During the spray-drying or extrusion stages, though, the pressure may be reduced, allowing the depressurized gas to bubble out of the particles of nutritional powder that are being formed at this stage. The exiting gas bubbles may leave a greater number of open pores or expanded open pores in the nutritional powder particles.

In some embodiments, after the nutritional powder is packaged into the pod, the pod is sealed and then stored under ambient conditions or under refrigeration for up to 36 months or longer, more typically from about 6 months to about 24 months. In some embodiments, a package is provided containing a plurality of nutritional powder pods. In some embodiments, a package containing a plurality of nutritional powder pods is prepared and stored.

The present invention has multiple aspects, illustrated by the following non-limiting examples.

### 5. EXAMPLES

The compositions used for the following examples are illustrated in Table 3, with specific ingredients for the control composition shown in Table 4.

TABLE 3
Compositions

| Composition | Components |
|---|---|
| 1 | Milk based, no HMOs added (Control) |
| 2 | Milk based, with 2′-fucosyllactose added (0.142 wt %) |

TABLE 4
Control Composition
lbs ingredient per 1000 lbs product

| Lactose | 119.72 | |
|---|---|---|
| Non-Fat Dry Milk | 76.59 | |
| Whey Protein Concentrate | 44.34 | |
| High Oleic Safflower Oil | 42.88 | |
| Soy Oil | 32.53 | |
| Coconut Oil | 29.70 | |
| GOS | 26.16 | |
| Potassium Citrate | 2.58 | |
| Calcium Carbonate | 1.41 | |
| ARA | 1.08 | |
| Nucleotide/Choline Premix | 422.48 | grams |
| DHA | 238.21 | grams |
| Ascorbic Acid | 224.73 | grams |
| Vitamin/Mineral Premix | 197.76 | grams |
| Lecithin | 188.77 | grams |
| Choline Chloride | 179.78 | grams |
| Ascorbyl Palmitate | 92.14 | grams |
| Vitamin A, D, E, and K premix | 86.74 | grams |
| Ferrous Sulfate | 85.40 | grams |
| Mixed Carotenoids | 80.00 | grams |
| Sodium Chloride | 76.41 | grams |
| Magnesium Chloride | 67.42 | grams |
| Mixed Tocopherols | 40.90 | grams |
| Tricalcium Phosphate | 30.56 | grams |
| Potassium Phosphate | 26.97 | grams |
| Potassium Chloride | 4.49 | grams |
| L-Carnitine | 1.39 | grams |
| Riboflavin | 561.81 | milligrams |

### Example 1

### Characterization of the Nutritional Powder

The nutritional powder described above was evaluated with regards to the size, surface area and shape of the particles comprising the powder, the porosity, thermal properties, bulk density, flowability, free fat content, and the wettability of the powder.

Particle Size and Shape. A study was conducted to evaluate the size of the nutritional powder particles, as well as their shape. Following the production of the nutritional powder, samples of said powder were collected and analyzed using laser diffraction. From this analysis, the particle size of the nutritional powder was provided as a distribution of the average particle size. Results are summarized in Table 5.

TABLE 5

| | | | 50% of | |
|---|---|---|---|---|
| | Mean | 10% of | particles | 90% of |
| | particle | particles | larger or | particles |
| | size | larger than | smaller than | smaller than |
| Composition | (μm) | (μm) | (μm) | (μm) |
| 1 | 92 | 15 | 81 | 182 |
| 2 | 77 | 10 | 68 | 156 |

The size and shape of the particles may further be examined via image analysis, for example, confocal microscopy and transmission electron microscopy. The particle shape and morphology are also assessed for aspect ratio via the aforementioned techniques. For example, the Malvern Morphologi G3 can be used to measure the size and shape of particles by the technique of static image analysis. There are three essential stages in the measurement process; sample preparation and dispersion (this step is critical to getting good results); spatial separation of individual particles and agglomerates. The Morphologi G3 has an integrated dry powder disperser which makes preparing dry powder samples easy and reproducible. The applied dispersion energy can be precisely controlled, enabling the measurement process to be optimized for a range of material types. Dispersion is achieved without explosively shocking the particles, avoiding damage to fragile particles while ensuring strongly agglomerated materials are dispersed. Effective dispersion of fibers can also be achieved. The instrument captures images of individual particles by scanning the sample underneath the microscope optics, while keeping the particles in focus. The instrument can illuminate the sample from below or above, while accurately controlling the light levels. Additionally, polarizing optics can be used to study birefringent materials.

In summary, these studies determine the size and shape of the nutritional powder particles, which in turn, provides information regarding the wettability and flow properties of the nutritional formula described above. It is expected that these results demonstrate particle size(s) and shape(s) that provides improved wettability and flow properties relative to a nutritional powder that does not have the same particle size and shape as the nutritional powder disclosed herein.

Particle Surface Area. In addition to the above examination of the size and shape of the nutritional powder particles, the surface area of the nutritional powder particles is investigated. Samples of the nutritional powder are analyzed via image analysis, for example, confocal microscopy and transmission electron microscopy to yield surface are of said particles. Alternatively, the surface area of the nutritional powder particles may be analyzed according to a Brunauer-Emmett-Teller (BET) multilayer gas adsorption method. In accordance with such methods, “adsorption” is the accumulation of atoms or molecules on the surface of a material. This adsorption is usually described through isotherms, as in, the amount of adsorbate on the adsorbent as a function of its pressure at constant temperature. This accumulation process creates a film of the adsorbate (the molecules or atoms being accumulated) on the surface of the adsorbent. Thus, the BET theory aims to explain the physical adsorption of gas molecules on a solid surface, and serves as the basis for an analysis technique or the measurement of the surface area of a material. Exemplary BET methods include, but are not limited, to those similar to or according to ISO-9277 (Determination of the specific surface area of solid by gas adsorption-BET method). The BET method may be performed on a Surface area and porosity analyzer using Krypton (Micromeretics TriStar II 3020).

In summary, these studies determine the surface area of the nutritional powder particles, which provides information regarding the wettability and flowability of the nutritional powder. It is expected that these results demonstrate a particle surface area that provides improved wettability and flowability relative to a nutritional powder that does not have the same particle surface area as the nutritional powder disclosed herein.

Thermal Properties. Studies were performed to investigate the thermal properties of the nutritional powder. Samples of the nutritional powder were transferred to a differential scanning calorimeter and examined for thermal properties over a temperature range of 0° C. to 120° C. The glass transition temperature of composition 1 was determined to be 52° C., while the glass transition temperature for composition 2 was determined to be 66° C.

In summary, these studies determined the thermal properties of the nutritional powder, such as the glass transition temperature and melting temperature, which are useful as a comparison for a new product at a standard moisture range.

Porosity. A study is conducted to examine the porosity of the particles comprised within the nutritional powder. Following the production of the nutritional powder, a sample is analyzed via a non-wetting based method on a porosimeter. Specifically, the method involves the intrusion of a non-wetting liquid (e.g., mercury) at high pressure into the powder. The pore size is based on the external pressure needed to force the liquid into a pore against the opposing force of the liquid's surface tension. The volume of the open pores and interstitial void is then divided by the envelope powder volume. Values for porosity can be provided in units of % (i.e. from 0-100%). Measurement of skim milk powder provides values of 40-75%. One exemplary spray dried infant formula may produce a value of about 57%.

In summary, these studies determine the porosity of the nutritional powder, which in turn, provides information regarding the wettability and flow properties of the nutritional powder and formula. It is expected that these results demonstrate a porosity that provides improved wettability and flow properties relative to a nutritional powder that does not have the same porosity as the nutritional powder disclosed herein.

Wettability. The wettability of the nutritional powder was also examined Wettability is defined as the period of time required for 1 teaspoon of powder to settle below the surface of water contained in a glass beaker. Wettability is designed to indirectly measure a powder's hydration characteristics. For example, a small amount of powder is dispersed on the surface of a small beaker of water. Particles which absorb water poorly will remain on top of the water for longer periods of time.

The method is as follows: 100 mL of tap water was added at the appropriate temperature to a glass beaker. The timing device was zeroed. One level teaspoon (˜2.0 grams) of powder was scooped. Holding the scoop over the center top of the beaker, the scoop was turned over and the powder was dropped into the tap water and the timer was started. When all the powder had sunk below the water surface, the timer was stopped. Time was recorded in seconds.

The wettability of compositions 1 and 2 is summarized in Table 6.

TABLE 6

| Composition | Wettability (sec) |
|---|---|
| 1 | 6 |
| 2 | 8 |

The wettability data indicates improved overall flow performance of the nutritional formula.

Flowability. A study is conducted to evaluate the flowability of the nutritional powder. After the nutritional powder is produced, a sample is transferred to a Brookfield powder flow tester. This instrument provides a flow factor and flow index of the nutritional powder sample.

Alternatively, the flowability index can be calculated by dividing the vibrated bulk density (VBD) by the loose bulk density (LBD), which were determined as described below. These results are summarized in Table 7.

TABLE 7

| | Flowability Index |
|---|---|
| Composition | (VBD/LBD) |
| 1 | 1.2 |
| 2 | 1.3 |

In summary, these studies determined the flowability of the nutritional powder, which is reflective of the cohesive forces within the powder. These results demonstrate a flowability that provides improved wettability and flow characteristics relative to a nutritional powder that does not have the same flowability as the nutritional powder disclosed herein.

Bulk Density. A study was conducted to investigate the density of the nutritional powder. Samples of the nutritional powder were measured for their bulk densities by specifically examining both loose bulk density and vibrated bulk density. The study was conducted as follows: a calibrated vibrated bulk density cylinder was obtained. The bottom section was labeled with the cylinder's volume. The tare weight of the bottom section of the cylinder was recorded. The top on the cylinder was placed and filled to near overflowing with the sample to be analyzed. A powder funnel may be used to simplify this task. While holding the cylinder over a waste can, the top section was removed. A spatula or the top section of the cylinder was used to strike off the excess sample so that it was smooth and flush with the top of the bottom section. A dry cloth was used to remove any powder clinging to the outside of the bottom section. The bottom section (Gross weight) was weighed.

The vibrated bulk density was calculated by following the sample preparation described in the loose bulk density. Then the cylinder was placed on the vibrated bulk density apparatus making sure it rested against the stop pins. The cylinder was clamped into place. The timer was set and preset for repeatable one minute cycles. This ensured a similar vibration cycle for all samples. After making sure that the vibrator apparatus was set at an amplitude of 5, the vibration cycle was started. When completed, the cylinder was unclamped and removed. While holding it over a waste can, the top section was removed. A spatula or the top section of the cylinder was used to strike off the excess sample so that it was smooth and flush with the top of the bottom section. A dry cloth was used to remove any powder clinging to the outside of the bottom section. The bottom section was weighed.

Results are summarized in Table 8.

TABLE 8

| | Loose bulk density | Vibrated bulk density |
|---|---|---|
| Composition | LBD (g/cc) | VBD (g/cc) |
| 1 | 0.40 | 0.50 |
| 2 | 0.37 | 0.48 |

In summary, both loose and vibrated bulk densities provided information on the nutritional powder, and may be important in the reconstitution of said powder. These results demonstrate powder bulk densities that provide improved wettability and reconstitution characteristics relative to a nutritional powder that does not have the same bulk density as the nutritional powder disclosed herein.

Free Fat Content. A study was performed to analyze the free fat content of the nutritional powder. The determination of fat free content was performed by stirring 2.00 g of nutritional powder in 80 mL of hexane (or another non-polar solvent such as petroleum ether) for 10 minutes, filtering the suspension through Whatman No. 41 paper into a tared beaker, evaporating the solvent at 80° C., and measuring the non-volatile residue gravimetrically. Results are summarized in Table 9.

TABLE 9

| | Free Fat (%, w/w, of |
|---|---|
| Composition | nutritional powder) |
| 1 | 0.37 |
| 2 | 0.45 |

These results demonstrate a fat free content that provides improved flowability relative to a nutritional powder that does not have the same fat free content as the nutritional powder disclosed herein.

Reconstitution. Generally, a nutritional powder reconstitution test was used to evaluate how thoroughly the nutritional powder was reconstituted under the operating conditions of a nutrient delivery system, and to determine a corresponding reconstitution rate.

Generally, according to this test, same size portions (e.g., portions of 2-5 g samples) were taken from the same batch of the nutritional powder to be tested. These portions were weighed both before and after drying (various type of drying can be utilized as long as each portion was dried using the same drying method, e.g., conventional drying techniques such as convection or IR can be utilized) to determine the initial moisture content of each portion (i.e., the weight lost to drying). The average initial moisture content (by weight) was determined by averaging the results from the multiple portions.

The weight of a resealable nutritional powder pod was measured both with and without a test sample of the nutritional powder enclosed therein to determine the initial weight of the sample of nutritional powder within the pod. Example amounts of the test samples of the nutritional powder were in the range from 2-150 grams.

The test system was configured to accommodate and operate under the operating conditions of a nutrient delivery system, as follows. The pressure within the pod, as well as the temperature of the water that contacts the nutritional powder and the amount of water flowing through the pod were controlled and measurable. For this test, the pod containing the test sample of the nutritional powder was inserted into the test system, and the system was set to deliver a certain amount of water (e.g., about 25-500 mL) at a certain temperature (e.g., in the range of 5-50° C.) under a certain pressure (e.g., 0.5-15 bar, or approximately 7-217 psia) into and through the pod. Under this test, the ratio of powder weight (grams) to water weight (grams) (where the density of water was taken to be 1 g/mL) was lower than 1:1 (e.g., 1:1.1, 1:1.2, 1:1.3, 1:2, 1:3, 1:5, etc.). In other words, relatively less powder (in grams) was used as compared to the amount (in grams) of water. A sufficiently large collection bottle was placed under the dispenser of the test system to receive the homogeneous liquid product output. The test system was started, and the homogeneous liquid product was collected in the collection bottle. It was intended that the test system may be a working nutrient delivery system operating under the above-specified conditions or a model system configured to simulate a nutrient delivery system and operating under the above-specified conditions.

Rate of Reconstitution: The rate of reconstitution is determined using the general test method and system described above, except that once the test system is started, aliquots are taken from the collection bottle or sample cups every 5 seconds until the product is fully dispensed. The total weight of reconstituted solids for each aliquot is determined in the same manner described above. The rate of reconstitution is determined by plotting, for each aliquot of liquid product collected, the weight of total reconstituted solids versus the collection time, thereby resulting in a “gram/ml·second” value.

In another embodiment, the reconstitution rate was determined by first turning on the microwave to warm up for 45 minutes. Funnel and tubing were set-up on the pod exit port of the nutrient delivery system, and 12 sample cups were labeled 1-12 accordingly. The nutrient delivery system was started at a water flow rate of 15 mL/second, and collection of the nutritional formula samples commenced as soon as formula entered the cup and was collected for 5 seconds. After 5 seconds, the tubing extending from the exit port was moved to the next cup (e.g., sample cup 2). This was continued until all of the nutritional formula had been dispensed from the nutrient delivery system.

Following completion of the nutrient delivery system run, an empty sample cup was tared, and each sample was weighed and recorded. Sample pads were placed in a microwave balance, and were tared (e.g., wait until the screen shows 0). A sample cup was taken and stirred for 5 seconds with a clean, unused syringe. Next, the syringe was filled with the sample and dispensed back into the cup. The syringe was filled again, and filled to a volume of 2 mL, except for samples 1-4, which were filled with 1 mL of sample. Next, the sample pads were removed from the microwave, and on the fuzzy side of one of the pads, sample was dispensed slowly from the syringe in a circular motion onto the center of the pad and moving outward. The other pad was placed on top of the aforementioned pad (fuzzy side down), and the two pads were pressed together. The pads were placed back into the microwave onto the balance, the microwave door closed, and the start button pressed to begin the process, which beeps and starts printing upon completion of the test. The percentage of total solids was recorded, and this was done for each sample.

All of the sample weights (g) were added up together to get the total weight. The sample weight was multiplied by the total solids (%) to get the sample total solids (g). Next, the sample solid total was multiplied by 1000 to convert the sample total solids to milligrams. Finally, the sample solids total (mg) was divided by the total weight (g) by the sample time (sec) to get the total solids (mg) per total weight (g) per sample time (sec).

Reconstituted Yield: The total solids in the final liquid product is measured using any standard drying technique (e.g., via a forced air oven or microwave drying technique) to remove the water from the final liquid product.

Next, the theoretical total solids content is determined according to the calculation below using an assumption that 100% of nutritional powder from the pod is delivered in the final liquid product.

Theoretical total solids=(total initial weight in grams of the powder sample in the model pod−average initial moisture in grams)/(water delivered in grams+total initial weight in grams of the powder sample in the model pod).

Finally, the reconstituted yield, which is the amount of reconstituted powder in the final liquid product, is determined by dividing the final liquid product total solids by the theoretical solids (i.e., reconstituted yield=final liquid product total solids/theoretical total solids). The reconstituted yield is reported as a number (e.g., 0.XX or as a percentage, e.g., XX %).

In another embodiment, reconstitution yield was determined by running the nutrient delivery system with a water flow rate of 120 mL over 5 seconds, and allowing the pod to remain within the system. One large sample cup was labeled with the run number, and a collection beaker or funnel was placed under the exit valve with the tubing set-up. Next, the accumulator was filled with approximately 120 mL of water and the nutrient delivery system was run again, with the original pod remaining within. The rinse water sample was collected within the sample cup. Similar to above in the reconstitution rate analysis, an empty sample cup was tared on a balance, and the rinse water sample weighed. In addition and like recited above, the steps used to determine total solids via microwave/pad analysis were used for the rinse water sample; however, 5 ml of sample was used relative to the smaller volumes listed above.

Then the percentage of total solids of the rinse water was multiplied by the grams of rinse water to get the grams of total solids of the rinse water. Next, the grams of total solids of the rinse water were divided by the percentage of total solids of the powder to get the grams of powder remaining in the pod. The grams of powder remaining in the pod were divided by the grams of powder put into the pod to get the ratio of powder remaining in the pod relative to powder put in the pod. Finally, the ratio of powder remaining in the pod relative to powder put in the pod was subtracted from 1 and multiplied by 100 to get the percentage of powder reconstituted.

The results for these studies are summarized in Table 10.

TABLE 10

| | Reconstitution | | Reconstitution rate (mg/g-sec) | | | |
|---|---|---|---|---|---|---|
| | Time | Yield | Start | 15 | | Conclusion |
| Composition | (sec) | (%) | of run | sec | 30 sec | of run |
| 1 | 40 | 98.5 | 10 | 2.0 | 1.2 | 0.3 |
| 2 | 45 | 99.6 | 12 | 0.3 | 0.6 | 0.1 |

Spectral Properties. A study was conducted to evaluate the spectral properties of the nutritional powder. The spectral characterization was assessed by transferring a sample of the nutritional powder to a spectrophotometer and measuring the Hunter L, a and b values. These values were dependent on the wettability, emulsion stability, and emulsion homogeneity of the nutritional formula, and indicate the lightness and color-opponent dimension of the nutritional formula. Spectral properties are provided in Table 11.

TABLE 11

| Composition | Hunter Color (L) | Hunter Color (a) | Hunter Color (b) |
|---|---|---|---|
| 1 | 90 | −2.5 | 19 |
| 2 | 90 | −2.8 | 17 |

The Hunter L, a, and b values of the nutritional formula are similar or improved relative to a nutritional powder lacking one or more of the components in the amounts described above.

### Example 2

### Characterization of the Nutritional Formula

The nutritional formula described above was evaluated with regards to foaming, gas entrapment/entrainment or density, viscosity, spectral properties, dispersibility and emulsion stability.

Dispersibility. A study was conducted to investigate the dispersibility of the nutritional formula. Following the production of the nutritional formula by a mechanical shaker, a sample was removed from and immediately poured through an 80 mesh screen. The receiving flask was filled approximately ¼ full with tap water to dislodge any particles that may remain in the flask, and poured through the screen. The screen was not rinsed with tap water. The particles remaining on the screen were rated using scaled photographs. If one lump that is 0.5 inch or greater remains on the screen, repeat the test. Each sample was tested in duplicate, and the average reported as the final result. Because the precision of the method is limited by the capability of the subjective rating, duplicate measurement was required.

In an additional experiment, the dispersibility of the nutritional formula was measured by the “80 Mesh Determination of Infant Formulas,” which describes the firmness and solubility of particles from the gel, sediment, and creaming within a product. This evaluation attempts to identify what product defects may lead to a clogged nipple.

This experiment employed either a 3″ U.S. Standard 80 mesh sieve, a 5″ U.S. Standard 80 mesh sieve, or a 8″ U.S. Standard 80 mesh sieve. The 5″ or 8″ sieve was used for samples in containers 11 oz. or greater. The 3″ sieve was used for samples in containers 8 oz. or smaller.

A flow of tap water was adjusted to a temperature of 110° F. The sieve was held over the sink and the sample (nutritional formula) was poured through the sieve. The sample container was then filled with water to rinse and poured through the sieve again. The water flow was fanned with an empty hand and the sieve rinsed for 20 seconds for samples that were a concentrated liquid, and rinsed for 3 seconds for sample that were ready-to-feed samples. The remaining particles on the screen were given a value of 1-6 using the following scale: 1—No particles; 2—First evidence of very small particles to a slight amount of small particles with a maximum size of approximately 1.0 mm; 3—Slight amount of small particles with a few moderate size particles; 4—Moderate amount of medium sized particles with a moderate amount of small particles; 5—A heavy amount of varying sized particles covering most of the sieve screen; 6—An excessive amount of any sized particles which cover the entire sieve screen and may plug the screen openings.

In another embodiment, the dispersibility of the nutritional formula was measured using a mesh sieve. For example, the nutritional formula was provided by the nutrient delivery system and poured through an 8 inch, 80 mesh sieve. Next, 100 mL of slightly warm water was added to the sample container and gently swirled. The residual rinse was also passed through the 80 mesh sieve, ensuring that the pour was distributed thoroughly over the area of the sieve. The total number of particles present on the sieve were measured using a mm stick and/or ruler. The size of the particles was stratified into groups consisting of less than 1 mm, 1 mm, 2 mm, 3 mm, 4 mm, 5 mm and greater than 5mm.

In another embodiment, dispersibility of the nutritional formula may be assessed after the nutritional powder is reconstituted via hand shaking. First, a tape was placed along a bench and/or table, which was used to mark the distance of the shake. The amount of powder was weighed to provide approximately an 8 oz serving, and the water bath was set to approximately 105° F. to 110° F. An amount of 210 mL of heated water was placed into an Avent baby bottle, and the preweighed powder was placed into the baby bottle. The baby bottle was capped, and the Metronome application was set to 242. Next, the baby bottle was held horizontally beside one end of the tape, a stop watch was started, and the baby bottle vigorously moved back and forth horizontally along the distance of the tape for 10 seconds. This distance and time roughly corresponds to a 40 count by Metronome beat. After this period of bottle shaking, the bottle cap was immediately removed and the contents poured through an 80 mesh sieve. The baby bottle was rinsed slightly, in order to remove any foam or clumps, and the rinse fluid poured through an 80 mesh sieve. Similar to above, the number and size of particles covering the surface of the sieve were measured and recorded. The size of the particles was stratified into groups consisting of less than 1 mm, 1 mm, 2 mm, 3 mm, 4 mm, 5 mm and greater than 5 mm.

Results are summarized in Table 12.

TABLE 12

| | | | | | | Hand consumer shake | | |
|---|---|---|---|---|---|---|---|---|
| | Hydration/solubility | | Appliance testing | | | testing | | |
| | Dispersibility | 80 | Dispersibility - quantitative | | | Dispersibility - quantitative | | |
| Composition | Subjective | Mesh | ≦1 mm | 2-4 mm | ≧5 mm | ≦1 mm | 2-4 mm | ≧5 mm |
| 1 | 2 | 1 | 7 | 4 | 0 | 4 | 5 | 1 |
| 2 | 3 | 1 | 61 | 18 | 1 | 55 | 25 | 7 |

The dispersibility indicates an improved flow formula relative to a nutritional formula lacking one or more of the components in the amounts described above.

Foaming. A study was conducted to evaluate the foaming of the nutritional formula described above. As the nutrient delivery system provides the nutritional formula at a water flow rate of 5 mL/second, the nutritional formula was captured within a graduated cylinder. The total volume of foam and liquid (mL) in the cylinder was measured at 0 minutes, 15 minutes and 30 minutes after being dispensed from the nutrient delivery system. Foaming is indicated by a number of different parameters, such as: total foam volume measured at the aforementioned listed intervals, and foaming ratio of the initial volume divided by the volume at the variable time points listed above. The foam ratio describes the foam dissipation over a variable time interval for a sample.

In another embodiment, the foaming procedure was performed by providing the nutritional formula from the nutrient delivery system, and immediately pouring the nutritional formula slowly down the side of a slightly tilted 250 mL graduated cylinder. Near the end of the pour, the container used to capture the nutritional formula was swirled and any remaining foam was transferred into the 250 mL graduated cylinder. The cylinder was set upright to determine where the layer of foam begins and ends. A flashlight may be used if necessary. The divisions on the cylinder that encompass the foam layer were counted and recorded, which was referred to as the initial time point. The foam layer was observed again at 15 minutes and 30 minutes, and the amount of foam at each time point was recorded in the manner as described above. It should be noted that as the foam dissipates there may be pockets of foam and/or bubbles clinging to the side of the cylinder. Only foam that was dense and was part of the bulk layer was counted towards the foam volume.

In another embodiment, foaming procedure was performed by providing the nutritional formula by reconstituting the nutritional powder via hand shaking. First, a tape was placed along a bench and/or table, which was used to mark the distance of the shake. The amount of powder was weighed to provide an 8 oz serving, and the water bath was set to approximately 105° F. to 110° F. An amount of 210 mL of heated water was placed into an Avent baby bottle, and the preweighed powder was placed into the baby bottle. The baby bottle was capped, and the Metronome application was set to 242. Next, the baby bottle was held horizontally beside one end of the tape, a stop watch was started, and the baby bottle vigorously moved back and forth horizontally along the distance of the tape for 10 seconds. This distance and time roughly corresponds to a 40 count by Metronome beat. After this period of bottle shaking, the bottle cap was immediately removed and the contents were immediately poured slowly down the side of a slightly tilted 250 mL graduated cylinder. Near the end of the pour, the container used to capture the nutritional formula was swirled and any remaining foam was transferred into the 250 mL graduated cylinder. The cylinder was set upright to determine where the layer of foam begins and ends. A flashlight may be used if necessary. The divisions on the cylinder that encompass the foam layer were counted and recorded, which was referred to as the initial time point. The foam layer was observed again at 15 minutes and 30 minutes, and the amount of foam at each time point was recorded in the manner as described above. It should be noted that as the foam dissipates there may be pockets of foam and/or bubbles clinging to the side of the cylinder. Only foam that was dense and was part of the bulk layer was counted towards the foam volume.

The results are summarized in Tables 13 and 14.

TABLE 13
Appliance Testing - Foam

| | initial | 15 min | 30 min | Entrained air | Foamability (ratio |
|---|---|---|---|---|---|
| Composition | (mL) | (mL) | (mL) | (%) | of foam to air) |
| 1 | 16 | 14 | 14 | 5 | 3 |
| 2 | 14 | 12 | 12 | 4 | 3 |

TABLE 14
Hand Consumer Shake Testing

| | initial | 15 min | 30 min | Entrained air | Foamability (ratio |
|---|---|---|---|---|---|
| Composition | (mL) | (mL) | (mL) | (%) | of foam to air) |
| 1 | 60 | 48 | 46 | 20 | 3 |
| 2 | 52 | 48 | 42 | 17 | 3 |

The nutritional formula displays reduced foaming relative to a nutritional formula lacking one or more of the components in the amounts described above. This reduced foaming, in turn, provides a decrease in negative side effects associated with foaming, e.g., gassiness, thereby providing an improved overall quality in the experience of consuming the nutritional formula described herein.

Gas Entrapment/Entrainment & Density. A study is conducted to evaluate and compare the density of the nutritional formula. Specifically, once the nutritional formula is prepared using a mechanical shaker, the density is assessed using an automated density meter (Mettler Toledo DE51).

In order to determine if entrapped air is responsible for any difference in formula density (relative to a nutritional formula lacking one or more of the components in the amounts described above), a subset of samples are transferred to a Biichner flask, with a stir bar, and sealed with a rubber stopper. The samples are placed under vacuum (˜25 in. Hg) for approximately 2 hours to remove air from product. Density measurements, as described above, are then repeated for degassed samples. Alternatively, samples may be degassed via centrifugation.

In another embodiment, entrapped/entrained air was measured using a PAPEC Squeezer. A compression piston full was removed to the end of a sample tube, and rotated one revolution. The sample, which was prepared using the nutrient delivery system using a water flow rate of 5 mL/second, was poured into the tube and the tube filled up to the beginning of the threaded area at the top of the tube, which was approximately 240 mL. The screw on the cap was replaced, and the bleed valve was confirmed to be open (e.g., arrows are pointing up and down). Next, the tube was slightly tilted with the brass bleed valve at the top, and the compression piston turned clockwise to dispel the air pocket. When bubbles appeared out of the bleed valve, the bleed valve was stopped and closed (e.g., arrows are horizontal). The compression piston was rotated clockwise, while counting the turns (e.g., 1 turn=360°) and fraction of turns, until the fluid rises to the back score mark on the pressure indicating tube. The number of turns was recorded, including the fraction of turns. Finally, the percentage of entrained air was calculated using the following formula:

Entrapped/Entrained Air=(total turns including fraction of turns)−0.5*2.2 (1)

The entrained air measurements provide information regarding the flow characteristics of the nutritional formula as well as information regarding any side effects that may be associated with consumption of the nutritional formula. The nutritional formula has an air entrainment that promotes positive side effects and negates negative side effects arising from the consumption of nutritional formulas that lack one or more of the components in the amounts described above.

Results are provided in Tables 13 and 14.

Viscosity. A study was conducted to investigate the viscosity of the nutritional formula. The nutritional formula was provided by a mechanical shaker. The viscosity was assessed by transferring a sample of the nutritional formula to a rheometer and measuring the viscosity of said formula. The viscosity of the nutritional formula provided by composition 1 was determined to be 4.0 cps, while the viscosity of the nutritional formula provided by composition 2 was determined to be 3.6 cps.

In summary, the viscosity provides information regarding the overall flow performance of the nutritional formula. The measured viscosity indicates an improved flow performance of the nutritional formula relative to a nutritional formula lacking one or more of the components in the amounts described above.

Spectral Properties. A study was conducted to evaluate the spectral properties of the nutritional formula. Once the nutritional formula was provided by a mechanical shaker, the spectral characterization was assessed by transferring a sample of the nutritional formula to a spectrophotometer and measuring the Hunter L, a and b values. These values were dependent on the wettability, emulsion stability, and emulsion homogeneity of the nutritional formula, and indicate the lightness and color-opponent dimension of the nutritional formula. Spectral properties are provided in Table 15.

TABLE 15

| Composition | Hunter Color (L) | Hunter Color (a) | Hunter Color (b) |
|---|---|---|---|
| 1 | 89 | −1.5 | 12.1 |
| 2 | 89 | −1.9 | 11.9 |

The Hunter L, a, and b values of the nutritional formula are similar or improved relative to a nutritional formula lacking one or more of the components in the amounts described above.

Emulsion stability. A study is conducted to evaluate the stability of the emulsions within the nutritional formula. The nutritional formula is provided by a mechanical shaker, hand shaking, or a nutrient delivery system as described above.

Specifically, the nutritional formula is analyzed for emulsion size using laser diffraction, wherein a refractive index of 1.462 is used for the dispersed phase and 1.332 is used for the continuous phase (water). Emulsion particle size within the nutritional formula is provided as a distribution of the average particle size. Particle size of the emulsion is measured at variable time points post production of the nutritional formula.

It is expected that the nutritional formula exhibits an improved emulsion stability relative to a nutritional formula lacking one or more of the components in the amounts described above.

Other Properties. The following additional properties were measured and results are summarized in Table 16: reconstitution density, water activity, moisture (%), soluble protein, and protein digestibility.

TABLE 16

| Compo- | water | moisture | soluble protein | Digestible protein |
|---|---|---|---|---|
| sition | activity | (%) | (% of total protein) | (% of total protein) |
| 1 | 0.20 | 2.8 | 51 | 97 |
| 2 | 0.27 | 3.8 | 59 | 97 |

### Example 3

### Sample Nutritional Powders

Examples of spray-dried nutritional powders that were and/or will be tested in the nutrient delivery systems of the present disclosure are provided in the Tables 17-19 below. All ingredient amounts are listed as kilogram per 1000 kilogram batch of product, unless otherwise specified.

TABLE 17
Exemplary formulations

| Ingredient | Ex. 1 | | Ex. 2 | | Ex. 3 | | Ex. 4 | | Ex. 5 | |
|---|---|---|---|---|---|---|---|---|---|---|
| Nonfat | 456.9 | | 456.9 | | 456.9 | | 456.9 | | 456.9 | |
| dry milk | | | | | | | | | | |
| Lactose | 259.0 | | 259.0 | | 259.0 | | 259.0 | | 259.0 | |
| High oleic | 93.9 | | 93.9 | | 93.9 | | 93.9 | | 93.9 | |
| sunflower oil | | | | | | | | | | |
| Soy oil | 70.4 | | 70.4 | | 70.4 | | 70.4 | | 70.4 | |
| Coconut oil | 67.1 | | 67.1 | | 67.1 | | 67.1 | | 67.1 | |
| 2′ fucosyl- | 0.7584 | | 0.7204 | | 0.6824 | | 0.7964 | | 0.8344 | |
| lactose | | | | | | | | | | |
| (2′FL) | | | | | | | | | | |
| Galacto- | 53.5 | | 53.5 | | 53.5 | | 53.5 | | 53.5 | |
| oligosac- | | | | | | | | | | |
| charides | | | | | | | | | | |
| (GOS) | | | | | | | | | | |
| Pro biotic | 1.0 | | 0.95 | | 0.90 | | 1.05 | | 1.10 | |
| Flavoring | 6.2 | | 6.2 | | 6.2 | | 6.2 | | 6.2 | |
| agent | | | | | | | | | | |
| Calcium | 4.8 | | 4.8 | | 4.8 | | 4.8 | | 4.8 | |
| carbonate | | | | | | | | | | |
| Potassium | 4.7 | | 4.7 | | 4.7 | | 4.7 | | 4.7 | |
| citrate | | | | | | | | | | |
| Oligo | 2.9 | | 2.9 | | 2.9 | | 2.9 | | 2.9 | |
| fructose | | | | | | | | | | |
| Ascorbic | 2.0 | | 2.0 | | 2.0 | | 2.0 | | 2.0 | |
| acid | | | | | | | | | | |
| Nucleotide/ | 1.8 | | 1.8 | | 1.8 | | 1.8 | | 1.8 | |
| Choline | | | | | | | | | | |
| Premix | | | | | | | | | | |
| ARA oil | 1.8 | | 1.8 | | 1.8 | | 1.8 | | 1.8 | |
| Vitamin/ | 1.5 | | 1.5 | | 1.5 | | 1.5 | | 1.5 | |
| Trace | | | | | | | | | | |
| Mineral | | | | | | | | | | |
| Premix | | | | | | | | | | |
| Sodium | 1.3 | | 1.3 | | 1.3 | | 1.3 | | 1.3 | |
| chloride | | | | | | | | | | |
| Lecithin | 1.2 | | 1.2 | | 1.2 | | 1.2 | | 1.2 | |
| Sodium | 982.2 | g | 982.2 | g | 982.2 | g | 982.2 | g | 982.2 | g |
| citrate | | | | | | | | | | |
| DHA oil | 882.1 | g | 882.1 | g | 882.1 | g | 882.1 | g | 882.1 | g |
| Magnesium | 477.4 | g | 477.4 | g | 477.4 | g | 477.4 | g | 477.4 | g |
| chloride | | | | | | | | | | |
| Vitamin A, | 314.7 | g | 314.7 | g | 314.7 | g | 314.7 | g | 314.7 | g |
| D3, E, K1 | | | | | | | | | | |
| Premix | | | | | | | | | | |
| Ascorbyl | 278.8 | g | 278.8 | g | 278.8 | g | 278.8 | g | 278.8 | g |
| Palmitate | | | | | | | | | | |
| Antioxidant | 137.3 | g | 137.3 | g | 137.3 | g | 137.3 | g | 137.3 | g |
| Tocopheryl | 32.0 | g | 32.0 | g | 32.0 | g | 32.0 | g | 32.0 | g |
| acetate | | | | | | | | | | |
| Beta- | 11.0 | g | 11.0 | g | 11.0 | g | 11.0 | g | 11.0 | g |
| carotene | | | | | | | | | | |
| 30% | | | | | | | | | | |
| Potassium | 2.5 | g | 2.5 | g | 2.5 | g | 2.5 | g | 2.5 | g |
| iodide | | | | | | | | | | |
| Riboflavin | 2.0 | g | 2.0 | g | 2.0 | g | 2.0 | g | 2.0 | g |
| Magnesium | 499.5 | mg | 499.5 | mg | 499.5 | mg | 499.5 | mg | 499.5 | mg |
| sulfate | | | | | | | | | | |

| | | | | | |
|---|---|---|---|---|---|
| Potassium | AN | AN | AN | AN | AN |
| phosphate | | | | | |
| dibasic | | | | | |
| Potassium | AN | AN | AN | AN | AN |
| chloride | | | | | |
| Tricalcium | AN | AN | AN | AN | AN |
| phosphate | | | | | |
| Potassium | AN | AN | AN | AN | AN |
| hydroxide | | | | | |
| Calcium | AN | AN | AN | AN | AN |
| hydroxide | | | | | |
| Sodium | AN | AN | AN | AN | AN |
| hydroxide | | | | | |
| Water | Q.S. | Q.S. | Q.S. | Q.S. | Q.S. |

AN = as needed

TABLE 18
Exemplary formulations

| Ingredient | Ex. 6 | | Ex. 7 | | Ex. 8 | | Ex. 9 | | Ex. 10 | |
|---|---|---|---|---|---|---|---|---|---|---|
| Water | Q.S. | | Q.S. | | Q.S. | | Q.S. | | Q.S. | |
| Corn syrup | 308.9 | | 308.9 | | 308.9 | | 308.9 | | 308.9 | |
| Maltodextrin | 297.1 | | 297.1 | | 297.1 | | 297.1 | | 297.1 | |
| Sucrose | 112.4 | | 112.4 | | 112.4 | | 112.4 | | 112.4 | |
| High Oleic | 84.9 | | 84.9 | | 84.9 | | 84.9 | | 84.9 | |
| sunflower oil | | | | | | | | | | |
| Sodium | 73.0 | | 73.0 | | 73.0 | | 73.0 | | 73.0 | |
| caseinate | | | | | | | | | | |
| Calcium | 50.2 | | 50.2 | | 50.2 | | 50.2 | | 50.2 | |
| caseinate | | | | | | | | | | |
| 2′fucosyl- | 0.7584 | | 0.7204 | | 0.6824 | | 0.7964 | | 0.8344 | |
| lactose | | | | | | | | | | |
| (2′FL) | | | | | | | | | | |
| Inulin, | 47.0 | | 47.0 | | 47.0 | | 47.0 | | 47.0 | |
| oligofructose | | | | | | | | | | |
| Soy oil | 38.3 | | 38.3 | | 38.3 | | 38.3 | | 38.3 | |
| Isolated soy | 35.9 | | 35.9 | | 35.9 | | 35.9 | | 35.9 | |
| protein | | | | | | | | | | |
| Milk protein | 16.3 | | 16.3 | | 16.3 | | 16.3 | | 16.3 | |
| isolate | | | | | | | | | | |
| Canola oil | 13.7 | | 13.7 | | 13.7 | | 13.7 | | 13.7 | |
| Sodium | 9.8 | | 9.8 | | 9.8 | | 9.8 | | 9.8 | |
| citrate | | | | | | | | | | |
| Potassium | 9.7 | | 9.7 | | 9.7 | | 9.7 | | 9.7 | |
| citrate | | | | | | | | | | |
| Tricalcium | 9.0 | | 9.0 | | 9.0 | | 9.0 | | 9.0 | |
| phosphate | | | | | | | | | | |
| Flavoring | 7.3 | | 7.3 | | 7.3 | | 7.3 | | 7.3 | |
| agent | | | | | | | | | | |
| Magnesium | 6.2 | | 6.2 | | 6.2 | | 6.2 | | 6.2 | |
| chloride | | | | | | | | | | |
| Potassium | 5.5 | | 5.5 | | 5.5 | | 5.5 | | 5.5 | |
| chloride | | | | | | | | | | |
| Choline | 1.7 | | 1.7 | | 1.7 | | 1.7 | | 1.7 | |
| chloride | | | | | | | | | | |
| Vitamin | 950.0 | g | 950.0 | g | 950.0 | g | 950.0 | g | 950.0 | g |
| premix | | | | | | | | | | |
| Ascorbic | 755.0 | g | 755.0 | g | 755.0 | g | 755.0 | g | 755.0 | g |
| acid | | | | | | | | | | |
| Vitamin/ | 465.0 | g | 465.0 | g | 465.0 | g | 465.0 | g | 465.0 | g |
| trace mineral | | | | | | | | | | |
| premix | | | | | | | | | | |
| Potassium | 215.9 | g | 215.9 | g | 215.9 | g | 215.9 | g | 215.9 | g |
| hydroxide | | | | | | | | | | |
| Potassium | 185.8 | g | 185.8 | g | 185.8 | g | 185.8 | g | 185.8 | g |
| phosphate | | | | | | | | | | |
| dibasic | | | | | | | | | | |
| Ascorbyl | 164.7 | g | 164.7 | g | 164.7 | g | 164.7 | g | 164.7 | g |
| palmitate | | | | | | | | | | |
| Antioxidant | 82.3 | g | 82.3 | g | 82.3 | g | 82.3 | g | 82.3 | g |
| Vitamin A, | 82.3 | g | 82.3 | g | 82.3 | g | 82.3 | g | 82.3 | g |
| D3, E, K1 | | | | | | | | | | |
| premix | | | | | | | | | | |
| Vitamin A | 16.5 | g | 16.5 | g | 16.5 | g | 16.5 | g | 16.5 | g |
| palmitate | | | | | | | | | | |
| Ferrous | 12.0 | g | 12.0 | g | 12.0 | g | 12.0 | g | 12.0 | g |
| sulfate | | | | | | | | | | |
| Beta | 5.5 | g | 5.5 | g | 5.5 | g | 5.5 | g | 5.5 | g |
| carotene | | | | | | | | | | |
| 30% | | | | | | | | | | |
| Vitamin | 1.0 | g | 1.0 | g | 1.0 | g | 1.0 | g | 1.0 | g |
| D3 oil | | | | | | | | | | |
| Potassium | 800.0 | mg | 800.0 | mg | 800.0 | mg | 800.0 | mg | 800.0 | mg |
| iodide | | | | | | | | | | |

| | | | | | |
|---|---|---|---|---|---|
| Citric acid | AN | AN | AN | AN | AN |
| Potassium | AN | AN | AN | AN | AN |
| hydroxide | | | | | |
| 40% | | | | | |
| Maltodextrin | AN | AN | AN | AN | AN |
| Magnesium | AN | AN | AN | AN | AN |
| sulfate | | | | | |
| Sodium | AN | AN | AN | AN | AN |
| chloride | | | | | |
| Calcium | AN | AN | AN | AN | AN |
| carbonate | | | | | |

AN = as needed

TABLE 19
Exemplary Formulations

| | | |
|---|---|---|
| Ingredient | Ex. 11 | Ex. 12 |

| Nonfat Milk | 692.9 | | 692.9 | |
|---|---|---|---|---|
| Lactose | 324.2 | | 324.2 | |
| Whey Protein Concentrate | 119.3 | | 119.3 | |
| High Oleic Safflower Oil | 115.5 | | 115.5 | |
| Soy Oil | 87.7 | | 87.7 | |
| Coconut Oil | 80.0 | | 80.0 | |
| Galacto-oligosaccharides | 38.8 | | — | |
| Fructo-oligosaccharides | — | | 17.9 | |
| Potassium Citrate | 6.93 | | 6.93 | |
| Calcium Carbonate | 3.78 | | 3.78 | |
| Nucleotide-Choline Premix | 2.51 | | 2.51 | |
| 2′fucosyl-D-Lactose | 1.63 | | 1.63 | |
| Ascorbic Acid | 1.34 | | 1.34 | |
| Vitamin/Mineral/Taurine Premix | 1.17 | | 1.17 | |
| Soy Lecithin | 1.12 | | 1.12 | |
| ARASCO Mortierella alpina Oil | 1.09 | | 1.09 | |
| DHASCO Crypthecodinium cohnii Oil | 1.09 | | 1.09 | |
| Choline Chloride | 1.06 | | 1.06 | |
| Ascorbyl Palmitate | 547.6 | g | 547.6 | g |
| Vitamin A, D3, E, K1 premix | 515.9 | g | 515.9 | g |
| Ferrous Sulfate | 494.9 | g | 494.9 | g |
| Carotenoid Premix | 475.1 | g | 475.1 | g |
| Sodium Chloride | 454.8 | g | 454.8 | g |
| Magnesium Chloride | 411.7 | g | 411.7 | g |
| Mixed Tocopherols (70%) | 241.8 | g | 241.8 | g |
| Tricalcium Phosphate | 189.8 | g | 189.8 | g |
| Potassium Phosphate Monobasic | 166.5 | g | 166.5 | g |
| Potassium Chloride | 33.8 | g | 33.8 | g |
| L-Carnitine | 8.40 | g | 8.40 | g |
| Riboflavin | 3.33 | g | 3.33 | g |

| | | |
|---|---|---|
| Potassium Hydroxide (processing aid) | as needed | as needed |

### Example 4

### Reconstitution of Formulations with HMOs

Composition 2 was tested to determine the reconstitution of HMOs including 2′-fucosyllactose as well as 3′-sialyllactose and 6′-sialyllactose (inherently present in nonfat milk proteins). Results regarding the HMO levels are provided in Table 20.

TABLE 20
Reconstituted HMO levels

| | Hand Shake Test | | | Appliance Test | | |
|---|---|---|---|---|---|---|
| | mg/kg | mg/L | % | mg/kg | mg/L | |
| Tested | reconstituted | reconstituted | powder | reconstituted | reconstituted | % powder |
| Level | product | product | (wt/wt) | product | product | (wt/wt) |
| 2′-FL | 174 | 179 | 0.142 | 179 | 184 | 0.146 |
| 6′-SL | 130 | 134 | 0.106 | 130 | 134 | 0.106 |
| 3′-SL | 80.2 | 82.6 | 0.065 | 84.2 | 86.7 | 0.069 |
| Total | 384 | 396 | 0.314 | 393 | 405 | 0.321 |
| HMOs | | | | | | |

Notably, there is no loss of HMO content when the nutritional powders are reconstituted on the appliance, compared to the control (hand shake) method.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A nutrient delivery system comprising:
(a) a pod; and
(b) a nutritional powder comprising a human milk oligosaccharide or a precursor thereof;
wherein the nutrient delivery system provides a nutritional formula comprising the human milk oligosaccharide or precursor thereof.

2. The nutrient delivery system of claim 1, wherein the nutritional powder is comprised within the pod.

3. The nutrient delivery system of claim 1, wherein the nutritional formula is a synthetic infant formula.

4. The nutrient delivery system of claim 1, wherein the human milk oligosaccharide or precursor thereof is selected from the group consisting of sialic acid, 3′-sialyllactose, 6′-sialyllactose, 2′-fucosyllactose, 3′-fucosyllactose, lacto-N-tetraose and lacto-N-neotetraose, and any combination thereof.

5. The nutrient delivery system of claim 4, wherein the human milk oligosaccharide or precursor thereof is a combination of human milk oligosaccharides selected from the group consisting of: a combination of 6′-sialyllactose and 3′-sialyllactose; a combination of 3′-fucosyllactose and sialic acid; a combination of 2′-fucosyllactose and 3′-fucosyllactose; a combination of 2′-fucosyllactose, 3′-sialyllactose, and 6′-sialyllactose; a combination of 3′-sialyllactose, 3′-fucosyllactose, and lacto-N-neotetraose; and a combination of 6′-sialyllactose, 2′-fucosyllactose, and lacto-N-neotetraose.

6. The nutrient delivery system of claim 5, wherein the combination of human milk oligosaccharides is present in the nutritional formula at a total concentration of from about 0.001 mg/mL to about 20 mg/mL.

7. The nutrient delivery system of claim 4, wherein the human milk oligosaccharide is 6′-sialyllactose.

8. The nutrient delivery system of claim 7, wherein the 6′-sialyllactose is present in the nutritional formula at a concentration of from about 0.001 mg/mL to less than 0.25 mg/mL.

9. The nutrient delivery system of claim 4, wherein the human milk oligosaccharide is 2′-fucosyllactose.

10. The nutrient delivery system of claim 9, wherein the 2′-fucosyllactose is present in the nutritional formula at a concentration of from about 0.001 mg/mL to less than 2.0 mg/mL.

11. The nutrient delivery system of claim 4, wherein the human milk oligosaccharide is 3′-fucosyllactose.

12. The nutrient delivery system of claim 11, wherein the concentration of 3′-fucosyllactose is from about 0.001 mg/mL to about 10 mg/mL.

13. The nutrient delivery system of claim 4, wherein the human milk oligosaccharide is lacto-N-neotetraose.

14. The nutrient delivery system of claim 13, wherein the lacto-N-neotetraose is present in the nutritional formula at a concentration of from about 0.001 mg/mL to less than 0.2 mg/mL.

15. The nutrient delivery system of claim 1, wherein the nutritional powder further comprises a probiotic.

16. The nutrient delivery system of claim 15, wherein the probiotic is of human infant origin.

17. The nutrient delivery system of claim 16, wherein the probiotic is a Bifidobacterium.

18-31. (canceled)

32. A reconstituted liquid nutritional product produced by using a nutrient delivery system, the nutrient delivery system comprising:
(a) a pod; and
(b) a nutritional powder comprising a human milk oligosaccharide or a precursor thereof;
wherein the nutrient delivery system provides a reconstituted liquid nutritional product comprising the human milk oligosaccharide or precursor thereof.

33. The reconstituted liquid nutritional product of claim 32, wherein the reconstituted liquid nutritional product comprises at least one of: a protein, a carbohydrate, and a fat.

34. The reconstituted liquid nutritional product of claim 32, wherein the reconstituted liquid nutritional product is an infant formula.
